SEQUENCE LISTING (69 sequences)

SEQ ID NO: 1 - protein, 633 aa, Bacillus thuringiensis
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLY

SEQ ID NO: 2 - DNA, 1902 bp, synthetic construct
  misc_feature 1..1902: note = cry2Aa coding sequence optimized
  source 1..1902: mol_type = other DNA
atgaacaacgtcttgaactccggtagaaccaccatctgcgacgcctataatgtcgtcgcccacgaccctttttcttttgaacataagtccttggacaccatccagaaagagtggatggagtggaagagaaccgaccactctttgtacgtcgctcctgtcgttggtactgtctcttctttcttgttgaagaaggttggttccttgatcggtaagagaattttgtccgagttgtggggtattatcttcccatccggttccaccaatttgatgcaggatattttgagagaaactgagcaattccttaatcagagattgaacaccgatactttggcccgtgtcaacgccgaattgattggtttgcaggccaacatcagagagttcaaccaacaggttgacaattttttgaaccctacccagaacccagtcccattgtccatcacctcctccgtcaacaccatgcagcagttgtttttgaacagattgccacagtttcagatccagggatatcagttgttgttgttgcctttgtttgcccaagccgccaacatgcacttgtcctttatcagagacgtcatccttaacgccgacgaatggggaatctctgctgctaccttgagaacctaccgtgactacttgcgtaactacactagagactactccaactactgcatcaacacttatcagactgcctttagaggtttgaacacccgtttgcacgacatgttggagttccgtacctacatgttcttgaacgtttttgaatacgtttctatttggtccttgtttaagtatcagtctttgatggtctcctctggagccaacctttacgcctctggttctggtcctcaacagactcagtcctttaccgcccaaaattggccattcttgtactccctttttcaagttaactctaactatattttgtccggtatctccggtaccagactttctatcaccttcccaaacatcggaggtcttccaggttccactaccactcactcccttaactctgctcgtgttaactactccggtggtgtctcttctggtttgattggtgccactaacttgaatcataacttcaactgctccactgtcttgcctccattgtccacccctttcgtccgttcctggttggactctggaactgacagagagggtgttgctacctctaccaactggcagaccgagtcctttcaaaccaccttgtccttgcgttgtggtgccttctctgccagaggtaattctaattactttccagattatttcattagaaacatttctggtgtcccacttgtcatccgtaacgaggacttgaccagaccattgcattacaaccagatcagaaatatcgaatccccatccggtaccccaggaggtgccagagcctacttggtttctgtccacaacagaaagaacaacatctacgctgccaacgagaacggaaccatgattcaccttgccccagaggattacaccggttttaccatctcccctattcacgccacccaagttaacaatcaaactagaacttttatttctgaaaagtttggtaaccaaggagactccttgcgtttcgagcaatctaacaccaccgccagatacaccttgagaggaaacggtaactcttataacttgtacttgagagtttcctccatcggtaactccactatccgtgtcaccatcaacggacgtgtttacactgtctccaacgtcaacactaccaccaacaacgacggagtcaatgataacggtgcccgtttctctgacatcaacatcggtaacattgttgcctccgacaataccaatgtcacccttgacatcaacgtcaccttaaattctggtaccccatttgacttgatgaacatcatgttcgtcccaaccaacttgccaccattgtactga

SEQ ID NO: 3 - protein, 622 aa, synthetic construct
  REGION 1..622: note = Cry1Ab
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQK

SEQ ID NO: 4 - DNA, 1869 bp, synthetic construct
  misc_feature 1..1869: note = cry1Ab coding sequence optimized
  source 1..1869: mol_type = other DNA
atggataacaaccctaatatcaatgagtgcatcccctacaattgcctcagcaatcctgaggtggaggtgctcgggggtgagaggatagagactgggtacacccctatcgacatcagcctcagcctcacccagtttctcctctctgagttcgttcctggggccgggtttgtgctcgggctcgtggatataatctgggggatcttcggtcctagccagtgggacgccttccttgtgcagatcgagcaacttatcaaccagaggatcgaggagtttgctaggaaccaggccatcagcaggctcgaggggctcagcaatctctaccaaatctacgccgagagctttagggagtgggaggctgatcccactaaccctgccctcagggaggagatgcgtattcaattcaatgacatgaacagcgccctcactactgccatccctctcttcgccgtgcaaaattaccaagtgcccctcctcagcgtgtacgttcaagccgccaatcttcacctcagcgtgcttagggatgtgagcgtgttcgggcaaaggtgggggttcgatgccgccactatcaacagcaggtacaatgacctcaccaggctcatcgggaattacactgatcacgccgtgaggtggtacaatactgggctcgagagggtgtgggggcctgacagcagggattggatcaggtacaaccagttcaggagggaattaactctcactgtgctcgacatcgtgagcctcttccctaattacgacagcaggacttaccctatcaggactgtgagccaactcaccagggagatctacaccaatcctgtgctcgagaatttcgacgggagctttagggggagcgcccaagggatcgaggggagcatcaggagccctcacctcatggacatcctcaacagcatcaccatctacaccgacgcccacaggggggagtactactggagcgggcaccaaatcatggccagccctgtggggtttagcgggcctgagtttacctttcctctctacgggactatggggaatgccgcccctcaacaaaggatcgtggcccaactcgggcaaggggtgtacaggactctcagcagcaccctctacagaaggcctttcaatatcgggatcaacaatcaacaactcagcgtgctcgacggtactgagtttgcctacgggactagcagcaatctccctagcgccgtgtacaggaagagcggtactgtggacagcctcgatgagatcccccctcagaataacaacgtgcctcctaggcaagggtttagccacaggctcagccacgtgagcatgttcaggagcgggtttagcaatagcagcgtgagcatcatcagggcccctatgtttagctggattcataggagcgccgagttcaataacatcatccctagcagccaaatcactcagatccctctcaccaagagcaccaatctcgggagcgggacttctgtggtgaaggggcctgggtttactgggggggatatcctcaggaggactagccctgggcaaatcagcaccctcagggtgaatatcactgcccctctctctcagaggtacagagtgaggatcaggtacgccagcaccactaatctccagttccacaccagcatcgatgggaggcctatcaaccaggggaattttagcgccaccatgagcagcgggagcaatctccaaagcgggagctttaggactgtggggttcaccacccctttcaatttcagcaacgggagcagcgtgttcactctcagcgcccacgtgtttaacagcgggaacgaggtgtacatcgacagaatcgagtttgtgcctgccgaggtgaccttcgaggccgagtacgatctcgagagggcccaaaagtga

SEQ ID NO: 5 - protein, 655 aa, synthetic construct
  REGION 1..655: note = Cry1Bb
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQK

SEQ ID NO: 6 - DNA, 1968 bp, synthetic construct
  misc_feature 1..1968: note = cry1Bb coding sequence optimized
  source 1..1968: mol_type = other DNA
atgaccagcaacaggaagaatgagaacgagataatcaacgcccttagcatccccactgttagcaacccctctactcagatgaatctcagccctgacgctaggatagaggatagcctctgtgtggccgaagttaataatatcgacccttttgttagcgccagcaccgtgcagaccgggatcaacatcgctgggcgtattttaggggtgcttggggtgccttttgccggtcagcttgccagcttctatagcttcctcgttggggaattatggcccagcggtcgtgatccttgggagatctttcttgagcacgttgaacagctcataaggcaacaggtgaccgaaaataccaggaataccgctatcgccagacttgaagggctcgggagggggtacaggagctaccaacaagccctcgagacctggctcgacaatagaaatgacgctaggagcaggagcatcatcctcgagagatacgtggccctcgagcttgacataaccaccgccatccccctttttaggatcaggaacgaggaggtgcccctccttatggtttacgcccaggctgccaatctccacctccttctcctcagagatgcctctctttttgggtctgagtggggtatggccagctctgacgtgaaccagtactaccaggagcaaatcaggtacaccgaggaatacagcaaccactgcgtgcaatggtacaataccgggcttaacaatctcagggggaccaacgccgaaagctggcttaggtacaaccaattcaggagggatctcactcttggggtgcttgacctcgtggccctctttcctagctacgacactagaacctaccccatcaatactagcgcccagctcactagggagatctacactgatcctataggtaggactaatgcccccagcggtttcgccagcaccaattggttcaataacaatgctcccagctttagcgctatagaggccgccatattcaggccccctcaccttctcgactttcctgagcagctcactatctacagcgccagcagcaggtggagcagcactcaacacatgaattactgggtggggcacaggcttaattttaggcctatcggggggaccctcaacactagcacccaagggctcaccaacaatactagcatcaaccccgtgactctccaattcacttctagggacgtgtacaggactgagagcaacgccgggaccaatatcctcttcactacccctgtgaacggggtgccttgggccagattcaacttcatcaaccctcagaacatctatgagaggggggccaccacttactctcaaccctaccaaggtgtgggtattcaactctttgatagcgagaccgaacttcctcccgagaccaccgagaggcccaactacgagagctatagccataggctcagccatatcgggcttatcatcgggaacactctcagagcccctgtgtatagctggactcataggagcgccgacaggaccaataccatcgggcccaatagaatcacccagatcccccttgttaaggccctcaatcttcatagcggggtgactgttgttggtgggcctgggtttactgggggggatatcctcaggaggactaacaccggtacctttggggacatcaggctcaatatcaatgtgcctctcagccagaggtacagggtgaggataagatacgccagcactaccgatctccagtttttcaccagaatcaatgggactaccgtgaatatcgggaatttcagcaggaccatgaacagaggggacaatctcgagtacaggagcttcaggaccgctgggtttagcactccttttaatttcctcaatgctcaaagcacctttactctcggggctcagagcttcagcaatcaggaagtgtacatcgatagagtggagttcgtgcctgccgaggtgacctttgaggctgagtacgatctcgagagggcccaaaagtga

SEQ ID NO: 7 - DNA, 21501 bp, synthetic construct
  misc_feature 1..21501: note = Triple Bt PP binary vector pBI121
  source 1..21501: mol_type = other DNA
tgagcgtcgcaaaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgccagaaggccgccagagaggccgagcgcggccgtgaggcttggacgctagggcagggcatgaaaaagcccgtagcgggctgctacgggcgtctgacgcggtggaaagggggaggggatgttgtctacatggctctgctgtagtgagtgggttgcgctccggcagcggtcctgatcaatcgtcaccctttctcggtccttcaacgttcctgacaacgagcctccttttcgccaatccatcgacaatcaccgcgagtccctgctcgaacgctgcgtccggaccggcttcgtcgaaggcgtctatcgcggcccgcaacagcggcgagagcggagcctgttcaacggtgccgccgcgctcgccggcatcgctgtcgccggcctgctcctcaagcacggccccaacagtgaagtagctgattgtcatcagcgcattgacggcgtccccggccgaaaaacccgcctcgcagaggaagcgaagctgcgcgtcggccgtttccatctgcggtgcgcccggtcgcgtgccggcatggatgcgcgcgccatcgcggtaggcgagcagcgcctgcctgaagctgcgggcattcccgatcagaaatgagcgccagtcgtcgtcggctctcggcaccgaatgcgtatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgcgttcaatcggaccagcggaggctggtccggaggccagacgtgaaacccaacatacccctgatcgtaattctgagcactgtcgcgctcgacgctgtcggcatcggcctgattatgccggtgctgccgggcctcctgcgcgatctggttcactcgaacgacgtcaccgcccactatggcattctgctggcgctgtatgcgttggtgcaatttgcctgcgcacctgtgctgggcgcgctgtcggatcgtttcgggcggcggccaatcttgctcgtctcgctggccggcgccagatctggggaaccctgtggttggcatgcacatacaaatggacgaacggataaaccttttcacgcccttttaaatatccgattattctaataaacgctcttttctcttaggtttacccgccaatatatcctgtcaaacactgatagttttacggccggccgaagctttaatgcggtagtttatcacagttaaattgctaacgcagtcaggcaccgtgtatgaaatctaacaatgcgctcatcgtcatcctcggcaccgtcaccctggatgcttaggcatagcttggttatgccggtactgccgggcctcttgcgggatggggatcgatactcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggctcatttccacacccaccttcctcgtcttctccattcgcttcgccagcgctcgagcggccgaagggtttttggttttggtggccgaggcatcaggaggtgcgtcttccgtttcgatttcggattcgtcgatttgttttcgtgcttcgtgttgattgacatggctgaatcgcgcggtggattgtgttcttttgtttgtttccgcaatgtatgatggatcggatgatcttgtttggatgtctgcctcgtgcttttgtttgggattctggctcgtggaaacgagcgctcgttttctcttgctttgtcgtcgaatcggttgaatctgaggatagcgttttagatctgtgtagtgattggatctgctccgaacgagttgttgattggaccgaccggtttcgagatgatcaatcgaaatcatttttaagcgctgaggtgattgtgagatgaatattatatggtacctacaggattgaagttaatttggtatagtcaaaacgattttcaactgcaattttgtttgggtatttcgatgacttaaatttatcatgaggttcttgttcttgtttggggttaggtttcttccttgaggaaattttgtttctggtgtctaaagccaaaaaaaaaaaaaaaaacaaaaacaaaaaacctcgaatttctgctcctttatctgtctttctagtatgatcgagtacatgggtgcatggaatgatttgtggcttcttttggcttttattttggcagttgtggttcttaaagaatatggactgaattttcgttgggtgccaacatgagtagcttcagaaactaattctctgaatgaatgttttgtgtgctagcaatcgatcggtgaaattctattgttgcaattttttgttatgataaatcacttttcttaagtacgagaatcattttatcctggacagaatcaagccttataattcaagcataatcccgggatgaacaacgtcttgaactccggtagaaccaccatctgcgacgcctataatgtcgtcgcccacgaccctttttcttttgaacataagtccttggacaccatccagaaagagtggatggagtggaagagaaccgaccactctttgtacgtcgctcctgtcgttggtactgtctcttctttcttgttgaagaaggttggttccttgatcggtaagagaattttgtccgagttgtggggtattatcttcccatccggttccaccaatttgatgcaggatattttgagagaaactgagcaattccttaatcagagattgaacaccgatactttggcccgtgtcaacgccgaattgattggtttgcaggccaacatcagagagttcaaccaacaggttgacaattttttgaaccctacccagaacccagtcccattgtccatcacctcctccgtcaacaccatgcagcagttgtttttgaacagattgccacagtttcagatccagggatatcagttgttgttgttgcctttgtttgcccaagccgccaacatgcacttgtcctttatcagagacgtcatccttaacgccgacgaatggggaatctctgctgctaccttgagaacctaccgtgactacttgcgtaactacactagagactactccaactactgcatcaacacttatcagactgcctttagaggtttgaacacccgtttgcacgacatgttggagttccgtacctacatgttcttgaacgtttttgaatacgtttctatttggtccttgtttaagtatcagtctttgatggtctcctctggagccaacctttacgcctctggttctggtcctcaacagactcagtcctttaccgcccaaaattggccattcttgtactccctttttcaagttaactctaactatattttgtccggtatctccggtaccagactttctatcaccttcccaaacatcggaggtcttccaggttccactaccactcactcccttaactctgctcgtgttaactactccggtggtgtctcttctggtttgattggtgccactaacttgaatcataacttcaactgctccactgtcttgcctccattgtccacccctttcgtccgttcctggttggactctggaactgacagagagggtgttgctacctctaccaactggcagaccgagtcctttcaaaccaccttgtccttgcgttgtggtgccttctctgccagaggtaattctaattactttccagattatttcattagaaacatttctggtgtcccacttgtcatccgtaacgaggacttgaccagaccattgcattacaaccagatcagaaatatcgaatccccatccggtaccccaggaggtgccagagcctacttggtttctgtccacaacagaaagaacaacatctacgctgccaacgagaacggaaccatgattcaccttgccccagaggattacaccggttttaccatctcccctattcacgccacccaagttaacaatcaaactagaacttttatttctgaaaagtttggtaaccaaggagactccttgcgtttcgagcaatctaacaccaccgccagatacaccttgagaggaaacggtaactcttataacttgtacttgagagtttcctccatcggtaactccactatccgtgtcaccatcaacggacgtgtttacactgtctccaacgtcaacactaccaccaacaacgacggagtcaatgataacggtgcccgtttctctgacatcaacatcggtaacattgttgcctccgacaataccaatgtcacccttgacatcaacgtcaccttaaattctggtaccccatttgacttgatgaacatcatgttcgtcccaaccaacttgccaccattgtactgataagagctcaccggtcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgagaattgcggccggccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgactagtggtacaattctcaacacaacatatacaaaacaaacgaatctcaagcaatcaagcattctacttctattgcagcaatttaaatcatttcttttaaagcaaaagcaattttctgaaaattttcaccatttacgaacgatagcctaggatgattgaacaggatggtcttcatgctggatctccagctgcatgggttgaacgactctttgggtatgactgggcacaacagaccataggttgctctgatgctgcagttttcaggcttagtgcacaagggagacctgttctcttcgtcaagactgacttgtccggagctttgaatgagcttcaggatgaggctgccagactttcatggcttgctacaactggtgttccttgcgctgcagttcttgacgttgttacagaggctggaagggattggttgcttcttggtgaagtgccaggacaggacttgctttctagtcatctggcaccagctgagaaggtgtctataatggctgatgccatgcgaagacttcacacacttgatcctgccacatgtcctttcgatcaccaggctaagcataggattgagagggctaggactagaatggaagcaggtttggtggaccaagacgatcttgatgaggagcatcaaggacttgctccagctgaacttttcgcaaggctcaaagctaggatgccagatggagaggatcttgttgtgactcacggagatgcttgcttgcccaacatcatggttgagaatgggaggttctctggattcatcgactgtggtagactcggagttgcagataggtaccaggacattgcacttgcaactcgtgacatcgctgaggaacttggaggtgaatgggcagatcgtttccttgtcctctatgggattgctgcacctgattcacagcgtatcgcattctaccgactgctcgatgagttcttctgaaccggtgaaatcaccagtctctctctacaaatctatctctctctattttctccataataatgtgtgagtagttcccagataagggaattagggttcttatagggtttcgctcacgtgttgagcatataagaaacccttagtatgtatttgtatttgtaaaatacttctatcaataaaatttctaattcctaaaaccaaaatccagtactaaaatccagatcacctaaagtccctatagatctttgtcgtgaatataaaccagacatgagacgactaaacctggagcccagacgccgttcgaagctagaagtaccgcttaggcaggaggccgttagggaaaagatgctaaggcagggttggttacgttgactcccccgtaggtttggtttaaatatgataaagtggacgaattcaagcttgcatgcctgcagggcgatcgccaaatattacaacaataaggagtaaacttcactttttgagaatgaagatttttatacagagttaaaaatattacatcttttcagtaatatctaaagtaacatgtaaaaaattgactctatgtaaatgaggaaattaattgtatctataacttcacaaaagattgactctgtaatgtgcggcatgtgatgtggcattagatagagtaacaagaaagaagttaactttgtttagtaaaatataacacacttttggttttataaagaataatacatcttattttccttaattaggaaaaaaagaaaaggatgtatagtcaaagtcgatcaatattattagtatttcacttgacattgcatgtaccgctcgcttgttatttaggggaaaaaaaaagggtaccatactcttgttatttttccttttttcgcgaggatggtcgcgtttggttgggctttgggcctatccctacccaactccacgacatcaaatgcaacccctgcaaatttctttccccatcccttccccatttgcccatattatttgcacatgtaatggaaatatctctcttcctaaatttaacaccacatcttacgtttcacgagtcaattttttattagagcttttacaccatttttttcattaatgatttcgggatatgagaagaacccttctctaaattcttctcccccccccccccccccccacccaaaaaaaataaaaataaaattgggggacattttctcatgttaaaggtccacaaaaagagaaataaaaataaaatccgattgaatacatgattgaattattataattctagggttgatgactacatgtgattgaaattgacagatggggcatcaatggaatggcttagatccctcaattcctcaacaccagtgcttacaaaggagtggggatcagctcgagagacttcaacttcaccagtcaccaccatcacacattcatcgtcctccctaggatgagataagatcgccgagagcccgccacgtggcgctgtcatagcactggtaacgataaggctagatcaataaccttccccccttatgtggccagcaagcggtaatgtcatcacctatcagaaccgacggccaggatccgcagcctgtcaacgccggggatttgtggccctaggatacgtgaagggggaccttgtgtttatatataggatgaatcccacagtggaagttccataagagagcatacaagttctcactcaccaaaggaagaagaagaagaagaagagggagaagaggcgcgtattgggatcccgggatgaccagcaacaggaagaatgagaacgagataatcaacgcccttagcatccccactgttagcaacccctctactcagatgaatctcagccctgacgctaggatagaggatagcctctgtgtggccgaagttaataatatcgacccttttgttagcgccagcaccgtgcagaccgggatcaacatcgctgggcgtattttaggggtgcttggggtgccttttgccggtcagcttgccagcttctatagcttcctcgttggggaattatggcccagcggtcgtgatccttgggagatctttcttgagcacgttgaacagctcataaggcaacaggtgaccgaaaataccaggaataccgctatcgccagacttgaagggctcgggagggggtacaggagctaccaacaagccctcgagacctggctcgacaatagaaatgacgctaggagcaggagcatcatcctcgagagatacgtggccctcgagcttgacataaccaccgccatccccctttttaggatcaggaacgaggaggtgcccctccttatggtttacgcccaggctgccaatctccacctccttctcctcagagatgcctctctttttgggtctgagtggggtatggccagctctgacgtgaaccagtactaccaggagcaaatcaggtacaccgaggaatacagcaaccactgcgtgcaatggtacaataccgggcttaacaatctcagggggaccaacgccgaaagctggcttaggtacaaccaattcaggagggatctcactcttggggtgcttgacctcgtggccctctttcctagctacgacactagaacctaccccatcaatactagcgcccagctcactagggagatctacactgatcctataggtaggactaatgcccccagcggtttcgccagcaccaattggttcaataacaatgctcccagctttagcgctatagaggccgccatattcaggccccctcaccttctcgactttcctgagcagctcactatctacagcgccagcagcaggtggagcagcactcaacacatgaattactgggtggggcacaggcttaattttaggcctatcggggggaccctcaacactagcacccaagggctcaccaacaatactagcatcaaccccgtgactctccaattcacttctagggacgtgtacaggactgagagcaacgccgggaccaatatcctcttcactacccctgtgaacggggtgccttgggccagattcaacttcatcaaccctcagaacatctatgagaggggggccaccacttactctcaaccctaccaaggtgtgggtattcaactctttgatagcgagaccgaacttcctcccgagaccaccgagaggcccaactacgagagctatagccataggctcagccatatcgggcttatcatcgggaacactctcagagcccctgtgtatagctggactcataggagcgccgacaggaccaataccatcgggcccaatagaatcacccagatcccccttgttaaggccctcaatcttcatagcggggtgactgttgttggtgggcctgggtttactgggggggatatcctcaggaggactaacaccggtacctttggggacatcaggctcaatatcaatgtgcctctcagccagaggtacagggtgaggataagatacgccagcactaccgatctccagtttttcaccagaatcaatgggactaccgtgaatatcgggaatttcagcaggaccatgaacagaggggacaatctcgagtacaggagcttcaggaccgctgggtttagcactccttttaatttcctcaatgctcaaagcacctttactctcggggctcagagcttcagcaatcaggaagtgtacatcgatagagtggagttcgtgcctgccgaggtgacctttgaggctgagtacgatctcgagagggcccaaaagtgataagagctcggcgcgccgcaagcttatgtcatggtggatcgtggggttaccctcgaagcggcatcgttccaaacctttcaattgtagctagatgtcgaatgatccatttttcccttattacccaagtcctgtgatatttgtgtacttttgagttcgcagatgcgttgcattaagacccgatttattctatgaatgaattcagctgtttgcttgcttgcttactttcgttgttgatgtcctccactgagtgcaaattgcttgatgatgccgggtgtgtccacgacgagtactgtttcttttggttatcgaaagatgacttcgttaatccttccaagctagatgttgaaggaattgcaatggatgctagccattcatgagcttacataatgaagtgattgagccaagcttcaaatggaggagaagaagggaaacatctgagcgacggtagagtaccttgaagaatctctagagttagacgtaagagtaaaagtgattgcttagttgcgaactagaactcgcctcgattccaaccgagcatgagtaggttgagtgtcagatctcgttgagctcgacaatcgactcgagcaacatcagctgcctttaaagccttttcacttataaatgaagtatcaagtggagtgcgatcgcgaattcaagctttaatgcggtagtttatcacagttaaattgctaacgcagtcaggcaccgtgtatgaaatctaacaatgcgctcatcgtcatcctcggcaccgtcaccctggatgcttaggcatagcttggttatgccggtactgccgggcctcttgcgggatggggatcgatcccccctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggcttcttgagatccttcaacaattaccaacaacaacaaacaacaaacaacattacaattactatttacaattacagtcgactctagaggatccccgggatggataacaaccctaatatcaatgagtgcatcccctacaattgcctcagcaatcctgaggtggaggtgctcgggggtgagaggatagagactgggtacacccctatcgacatcagcctcagcctcacccagtttctcctctctgagttcgttcctggggccgggtttgtgctcgggctcgtggatataatctgggggatcttcggtcctagccagtgggacgccttccttgtgcagatcgagcaacttatcaaccagaggatcgaggagtttgctaggaaccaggccatcagcaggctcgaggggctcagcaatctctaccaaatctacgccgagagctttagggagtgggaggctgatcccactaaccctgccctcagggaggagatgcgtattcaattcaatgacatgaacagcgccctcactactgccatccctctcttcgccgtgcaaaattaccaagtgcccctcctcagcgtgtacgttcaagccgccaatcttcacctcagcgtgcttagggatgtgagcgtgttcgggcaaaggtgggggttcgatgccgccactatcaacagcaggtacaatgacctcaccaggctcatcgggaattacactgatcacgccgtgaggtggtacaatactgggctcgagagggtgtgggggcctgacagcagggattggatcaggtacaaccagttcaggagggaattaactctcactgtgctcgacatcgtgagcctcttccctaattacgacagcaggacttaccctatcaggactgtgagccaactcaccagggagatctacaccaatcctgtgctcgagaatttcgacgggagctttagggggagcgcccaagggatcgaggggagcatcaggagccctcacctcatggacatcctcaacagcatcaccatctacaccgacgcccacaggggggagtactactggagcgggcaccaaatcatggccagccctgtggggtttagcgggcctgagtttacctttcctctctacgggactatggggaatgccgcccctcaacaaaggatcgtggcccaactcgggcaaggggtgtacaggactctcagcagcaccctctacagaaggcctttcaatatcgggatcaacaatcaacaactcagcgtgctcgacggtactgagtttgcctacgggactagcagcaatctccctagcgccgtgtacaggaagagcggtactgtggacagcctcgatgagatcccccctcagaataacaacgtgcctcctaggcaagggtttagccacaggctcagccacgtgagcatgttcaggagcgggtttagcaatagcagcgtgagcatcatcagggcccctatgtttagctggattcataggagcgccgagttcaataacatcatccctagcagccaaatcactcagatccctctcaccaagagcaccaatctcgggagcgggacttctgtggtgaaggggcctgggtttactgggggggatatcctcaggaggactagccctgggcaaatcagcaccctcagggtgaatatcactgcccctctctctcagaggtacagagtgaggatcaggtacgccagcaccactaatctccagttccacaccagcatcgatgggaggcctatcaaccaggggaattttagcgccaccatgagcagcgggagcaatctccaaagcgggagctttaggactgtggggttcaccacccctttcaatttcagcaacgggagcagcgtgttcactctcagcgcccacgtgtttaacagcgggaacgaggtgtacatcgacagaatcgagtttgtgcctgccgaggtgaccttcgaggccgagtacgatctcgagagggcccaaaagtgataagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatatcaactcccctatccattgctcaccgaatggtacaggtcggggacccgaagttccgactgtcggcctgatgcatccccggctgatcgaccccagatctggggctgagaaagcccagtaaggaaacaactgtaggttcgagtcgcgagatcccccggaaccaaaggaagtaggttaaacccgctccgatcaggccgagccacgccaggccgagaacattggttcctgtaggcatcgggattggcggatcaaacactaaagctactggaacgagcagaagtcctccggccgccagttgccaggcggtaaaggtgagcagaggcacgggaggttgccacttgcgggtcagcacggttccgaacgccatggaaaccgcccccgccaggcccgctgcgacgccgacaggatctagcgctgcgtttggtgtcaacaccaacagcgccacgcccgcagttccgcaaatagcccccaggaccgccatcaatcgtatcgggctacctagcagagcggcagagatgaacacgaccatcagcggctgcacagcgcctaccgtcgccgcgaccccgcccggcaggcggtagaccgaaataaacaacaagctccagaatagcgaaatattaagtgcgccgaggatgaagatgcgcatccaccagattcccgttggaatctgtcggacgatcatcacgagcaataaacccgccggcaacgcccgcagcagcataccggcgacccctcggcctcgctgttcgggctccacgaaaacgccggacagatgcgccttgtgagcgtccttggggccgtcctcctgtttgaagaccgacagcccaatgatctcgccgtcgatgtaggcgccgaatgccacggcatctcgcaaccgttcagcgaacgcctccatgggctttttctcctcgtgctcgtaaacggacccgaacatctctggagctttcttcagggccgacaatcggatctcgcggaaatcctgcacgtcggccgctccaagccgtcgaatctgagccttaatcacaattgtcaattttaatcctctgtttatcggcagttcgtagagcgcgccgtgcgtcccgagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtgtctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaattatgatagaatttacaagctataaggttattgtcctgggtttcaagcattagtccatgcaagtttttatgctttgcccattctatagatatattgataagcgcgctgcctatgccttgccccctgaaatccttacatacggcgatatcttctatataaaagatatattatcttatcagtattgtcaatatattcaaggcaatctgcctcctcatcctcttcatcctcttcgtcttggtagctttttaaatatggcgcttcatagagtaattctgtaaaggtccaattctcgttttcatacctcggtataatcttacctatcacctcaaatggttcgctgggtttatcgcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 8 - DNA, 7267 bp, Eucalyptus grandis
  source 1..7267: mol_type = genomic DNA
gcaagaacccctttttcaaccctatgaagtagcatcataaggattttcaataagtatcattctaaatgtgttgcaagcaagtagagtgtttaaggtcgactttacaaggttttgatgttttctaggatatttggagaatgaaatgttgatttagcggcatctctctttgatgaaaaggagaggtagggttcaaatagggtgcagtaatagggtgcagtaaggaaaatctgatttatatgaaaatcgcatacatcttctttttcttaatcgggcaaaaaacaaaaaggatctatagtcaaagtcaatcaatataattagtatttcacttaacatagaacaataattctcaagaaaaacattgagattcccctcgtactaatcataaaaatcaaactaatctgaattatctgaaaataatcctcacaaaaaagtaattgaaatgtttttgtaatagaaaaccaggtaccattctcttgtcatctccacttttttttcttgaggatggtcgttgggttgggctttgggcctatctaaatgcgccctgaaccttactctctcatcagcactagcaaagctatacaaaatgaaaccgagttccgagatttccagctcaaaaactggagatgttctgttcgaatgttaccgattgcatgcgaactattctaggtcaaagccacttacacaaagtgtgtccaaataatcgaatcctcctgttcaaaaataaataaataaataatcgaatcctcacgccattcacctagattaaaaaagacaaataacaaaaagaaaattgctccaattctagggatgaacgtgattagatatctagattttctctatcataatcttccattcttagtccagtaaagatgacagtgattttaagtgaatgttttatcaacatttactttttcaagagaatgtattcaccacaatcttccttattgaacacaaacagcatgagtagatgtcggataggagagcaacatatggtcaaaattgacagcaaaaggaaagttatagacctattgcatttttcaataaaaataaattgtatatatactaattaagtcatgaaacttttaattataccaattgagcattaaaacctttgacaatttaccaatatccagtcaatttttgtcagaatcgctaacatggacactattgtcctacatagtgtggcctaggctgacatcaataatttttttaaacttcttattaattttttcttttctttttttcctcaattatgggttgacgactgccacaggctctatgtgagggcctccatgcccttgtcagccatgggcgagggcatccagtccttacctagatccaagagaggactgcgactttcgcctattggccaatgaggtccacaatgatgcccttgcttggtttgggcaagggccatgatacaattgcaagtgatgagcatctagctcttggtgagggcatggacgccctcgcccatgcttgcgagggccacgacgccctcacctatggctaataatggttggctgatgaccttgctatcctcgagttggtaaataaaaaataaaataaaataaaattatgcctatgttagtgttggttgtgtcatgtaggtcggccgctcatctatgttagggattttcaaccaatataagccacatagactacactggcaacttgtcaaaaaaatttaggactcaattagtacgaaatgtttagaattgaattagcataaaattgttatattaagacttctctccataattgactatatgactattttcgggtacttggttttggaccccatcactttggcaaggacagaggccttcagccaacacgctagcaatggcatatgagtctttccagttagacaaacattgaagacttaattgactaaaataatattttaacgacttgattgctcagcggtattgaaatatactcgataacctggaaataaccgagaaagtccgattgtaataatttattattaggttggaatcgttggataatataagatgcaaagtgtaatcatattgtactcgatgatttaggcaaaaatgcaatctgggagaaagatatttttcatgcaattggctccggaagattaattagcttttagcaatttataagtgtactaaaaacactagtagtttactaaaatatgaaatgcaaattttatggaatatactaaaagtgactcttggacactatgtgtactcagttttaatgaaatgaatattttctctcataaataaaaatctaagagttttatcaagaaaaactaaaaagtttggaggcaaatattacaacaataaggagtaaacttcactttttgagaatgaagatttttatacagagttaaaaatattacatcttttcagtaatatctaaagtaacatgtaaaaaattgactctatgtaaatgaggaaattaattgtatctataacttcacaaaagattgactctgtaatgtgcggcatgtgatgtggcattagatagagtaacaagaaagaagttaactttgtttagtaaaatataacacacttttggttttataaagaataatacatcttattttccttaattaggaaaaaaagaaaaggatgtatagtcaaagtcgatcaatattattagtatttcacttgacattgcatgtaccgctcgcttgttatttaggggaaaaaaaaagggtaccatactcttgttatttttccttttttcgcgaggatggtcgcgtttggttgggctttgggcctatccctacccaactccacgacatcaaatgcaacccctgcaaatttctttccccatcccttccccatttgcccatattatttgcacatgtaatggaaatatctctcttcctaaatttaacaccacatcttacgtttcacgagtcaattttttattagagcttttacaccatttttttcattaatgatttcgggatatgagaagaacccttctctaaattcttctcccccccccccccccccccacccaaaaaaaataaaaataaaattgggggacattttctcatgttaaaggtccacaaaaagagaaataaaaataaaatccgattgaatacatgattgaattattataattctagggttgatgactacatgtgattgaaattgacagatggggcatcaatggaatggcttagatccctcaattcctcaacaccagtgcttacaaaggagtggggatcagctcgagagacttcaacttcaccagtcaccaccatcacacattcatcgtcctccctaggatgagataagatcgccgagagcccgccacgtggcgctgtcatagcactggtaacgataaggctagatcaataaccttccccccttatgtggccagcaagcggtaatgtcatcacctatcagaaccgacggccaggatccgcagcctgtcaacgccggggatttgtggccctaggatacgtgaagggggaccttgtgtttatatataggatgaatcccacagtggaagttccataagagagcatacaagttctcactcaccaaaggaagaagaagaagaagaagagggagaagatggcctcctcaatgctctcctcggctgctgtcgccgccgtcaagagcaccagcccggttcaggctgccatggtggctccgttcaccggcctcaagtctgcctcggccttcccggtcacccgcaagagcaatgacatcacctctctccccagcaacggtggaagagtgcaatgcatgcaggtatactcgtctagctttagcttacagaatcatttctcggcactcgaaacaattcaggcttagatcgatttctacaacacgtgcctttagatggaagttctcatgagtagccatagttccagctgacatggaagaacgttctctgtgctggtgtgcataaaaaaggtgggagtgaatctggccagcgtcgtgagcgcgagttgtcgggcccattcttcattagaatcaaccggaagttaattccattttcgtgctgtttgtagaaaatgaacatcttaggagaatttcctaacgacgattctagtcagtgaggttattagcagcaacacatgtcctgaatgtcctgaataagttgagacttacgtcgatgcacatgggcaggtgtggccaccggtcgggctgaagaagttcgagacgctgtcctaccttcctcccctgactcccacacaattggcgaaggaaatcgactacctgctccggtcgggctgggttccttgcttggaatttgagactgaggtccttgtcttcaacaccctttttcaatttcaagttcttgacacaagtcgcaacgccattatgcaattgctttgcctaggtatatgatctttaactcgtagtacatattgtgatggaatggtgggatttcgcttttttgtttatgcagaagggcttcgtctaccgtgagcaccaccactccccggggtactacgatgggagatactggaccatgtggaagctgcccatgttcgggtgcaccgactcggcccaggtgttgaaggagcttgaggagtgcaagaaggcttaccccaccgctttcgtcagaatcatcggcttcgacaacaagcgtcaagtgcagtgcatcagtttcatcgccttcaagcccccgggcttctaagcaagcttatgtcatggtggatcgtggggttaccctcgaagcggcatcgttccaaacctttcaattgtagctagatgtcgaatgatccatttttcccttattacccaagtcctgtgatatttgtgtacttttgagttcgcagatgcgttgcattaagacccgatttattctatgaatgaattcagctgtttgcttgcttgcttactttcgttgttgatgtcctccactgagtgcaaattgcttgatgatgccgggtgtgtccacgacgagtactgtttcttttggttatcgaaagatgacttcgttaatccttccaagctagatgttgaaggaattgcaatggatgctagccattcatgagcttacataatgaagtgattgagccaagcttcaaatggaggagaagaagggaaacatctgagcgacggtagagtaccttgaagaatctctagagttagacgtaagagtaaaagtgattgcttagttgcgaactagaactcgcctcgattccaaccgagcatgagtaggttgagtgtcagatctcgttgagctcgacaatcgactcgagcaacatcagctgcctttaaagccttttcacttataaatgaagtatcaagtggagttttaaactaccatttgcttcaagagttagcaagtaaaagttaacgcttgacctcaagtataagtcatcttgaactaaaattgagtatttagtgctattactgcattctgagtgtcaagccttgaatattctcacgtgtagtcaagtgctgcgacgtccggacatgaatgggctacaccctcagtaactccagatacaataacaccgttggcactcgaggcaataatggatctacttcaccagaaaaagcacacccattctagcaccattgaagcttctgtggctgaactggtgccggcttgcaactcgattcttctaagcatcaatttatcttgtaacgcatacatcaaaatgagaaagtttgagggacaaatagagagagagagagagagagagatagagccataaaacactatctaggataaacatttttcaaagaggaaaagttgccggagaacgtctccaggactgtcgtcaagtataaagtaaggcatgaggtagacggaaatttcttgccttgatcctatgtagattaaggtacccggaaggggctcttgatctctaaaaggatctcctatagctactcgatctcaatcgtagatctctctctacttgagtactcataacagtatataaaactcttattgtgagtctcctcacatcatccaacaagtctgcctacacggcatccatgatggaccgaggtcaacataagatttgatgaaagatccacataacagtatatagaactagaaaactagttgtgcactagtgaaacaaccaaatgacataaattttcaacaagctcatttgctgggaaactgcggactatggagatttcctcccttttgttcttttttatctttctgtttttttcattgggattcaattctaccctcatattaggagtgacctgtttaatgatggattggttctcaacggagaacctaaaccttacccaattagaatcgcttacagatttttggaaatagttggatttagggtagatgccttcattactgatcctcggtgatggccacgattggccacaagcaagagccgaccttcccagcctcaagccaaactcaaactagccacatttgacaatgtggagccttgcccaaggctaaagagggtcaacctcactgagatttggcgaacttgagccttgctcaaggccagcaaggttggccctcaccaaccttttccaatggctagtaatcatcaagcctatcattggttgttgctaagccttggcgaccgacgaaagaaaaaattaaaaatatataaaaatattggttatatcacataggacgtttggcgtttatgttcgtaatttccaatcaaatttgattggaatgactaaattaacacattattataagatttagaactcgattagtgtaattaaaagatttataactaaattatcacaagcgtaataaatttatgattttttttttaaaatttatccatgaacttgcttagctcgctgagaacaagatcaatgctcacccttacctcatgtcatcgtcatttgtacgtttcatcctccattatttatttatttattttgagaaatgagcctcacaaacttgctcggctcatgcaacgaaacttttacatcatgtcggaggatttttgacagaatagataggtatcttgaaattataaatggtttgatataagaagactttatcaaaattgcccacatgactccacttgtcctatcatttcatgtccgtgtcgtggtgtcagatgtggaaaacaaaagattgataaaagataaaaatatggaaaaataatacaaatgatctatgaattttgacctaatattttatattgttcttaaacttttaatttatttaatatgatccccgaactattaataaatatttaatgtaatctttccatttggttaaacaaccaactgtatgtttttttataatgtggcttattatacaatgagttgaatttctaaaaatgacaagtcaacaaatc

SEQ ID NO: 9 - DNA, 67 bp, synthetic construct
  misc_feature 1..67: note = Omega 5 UTR sequence Tobacco Mosaic Virus
  source 1..67: mol_type = other DNA
agatccttcaacaattaccaacaacaacaaacaacaaacaacattacaattactatttacaattaca

SEQ ID NO: 10 - DNA, 69 bp, synthetic construct
  misc_feature 1..69: note = 5' UTR of RbcS
  source 1..69: mol_type = other DNA
gaagttccataagagagcatacaagttctcactcaccaaaggaagaagaagaagaagaagagggagaag

SEQ ID NO: 11 - DNA, 600 bp, synthetic construct
  misc_feature 1..600: note = 3 UTR and terminator of RbcS
  source 1..600: mol_type = other DNA
gcaagcttatgtcatggtggatcgtggggttaccctcgaagcggcatcgttccaaacctttcaattgtagctagatgtcgaatgatccatttttcccttattacccaagtcctgtgatatttgtgtacttttgagttcgcagatgcgttgcattaagacccgatttattctatgaatgaattcagctgtttgcttgcttgcttactttcgttgttgatgtcctccactgagtgcaaattgcttgatgatgccgggtgtgtccacgacgagtactgtttcttttggttatcgaaagatgacttcgttaatccttccaagctagatgttgaaggaattgcaatggatgctagccattcatgagcttacataatgaagtgattgagccaagcttcaaatggaggagaagaagggaaacatctgagcgacggtagagtaccttgaagaatctctagagttagacgtaagagtaaaagtgattgcttagttgcgaactagaactcgcctcgattccaaccgagcatgagtaggttgagtgtcagatctcgttgagctcgacaatcgactcgagcaacatcagctgcctttaaagccttttcactt

SEQ ID NO: 12 - DNA, 1902 bp, Bacillus thuringiensis
  source 1..1902: mol_type = genomic DNA
atgaataatgtattgaatagtggaagaacaactatttgtgatgcgtataatgtagtagcccatgatccatttagttttgaacataaatcattagataccatccaaaaagaatggatggagtggaaaagaacagatcatagtttatatgtagctcctgtagtcggaactgtgtctagttttttgctaaagaaagtggggagtcttattggaaaaaggatattgagtgaattatgggggataatatttcctagtggtagtacaaatctaatgcaagatattttaagggagacagaacaattcctaaatcaaagacttaatacagatacccttgctcgtgtaaatgcagaattgatagggctccaagcgaatataagggagtttaatcaacaagtagataattttttaaaccctactcaaaaccctgttcctttatcaataacttcttcggttaatacaatgcagcaattatttctaaatagattaccccagttccagatacaaggataccagttgttattattacctttatttgcacaggcagccaatatgcatctttcttttattagagatgttattcttaatgcagatgaatggggtatttcagcagcaacattacgtacgtatcgagattacctgagaaattatacaagagattattctaattattgtataaatacgtatcaaactgcgtttagagggttaaacacccgtttacacgatatgttagaatttagaacatatatgtttttaaatgtatttgaatatgtatccatttggtcattgtttaaatatcagagtcttatggtatcttctggcgctaatttatatgctagcggtagtggaccacagcagacacaatcatttacagcacaaaactggccatttttatattctcttttccaagttaattcgaattatatattatctggtattagtggtactaggctttctattaccttccctaatattggtggtttaccgggtagtactacaactcattcattgaatagtgccagggttaattatagcggaggagtttcatctggtctcataggggcgactaatctcaatcacaactttaattgcagcacggtcctccctcctttatcaacaccatttgttagaagttggctggattcaggtacagatcgagagggcgttgctacctctacgaattggcagacagaatcctttcaaacaactttaagtttaaggtgtggtgctttttcagcccgtggaaattcaaactatttcccagattattttatccgtaatatttctggggttcctttagttattagaaacgaagatctaacaagaccgttacactataaccaaataagaaatatagaaagtccttcgggaacacctggtggagcacgggcctatttggtatctgtgcataacagaaaaaataatatctatgccgctaatgaaaatggtactatgatccatttggcgccagaagattatacaggatttactatatcgccaatacatgccactcaagtgaataatcaaactcgaacatttatttctgaaaaatttggaaatcaaggtgattccttaagatttgaacaaagcaacacgacagctcgttatacgcttagagggaatggaaatagttacaatctttatttaagagtatcttcaataggaaattcaactattcgagttactataaacggtagagtttatactgtttcaaatgttaataccactacaaataacgatggagttaatgataatggagctcgtttttcagatattaatatcggtaatatagtagcaagtgataatactaatgtaacgctagatataaatgtgacattaaactccggtactccatttgatctcatgaatattatgtttgtgccaactaatcttccaccactttattaa

SEQ ID NO: 13 - DNA, 3468 bp, Bacillus thuringiensis
  source 1..3468: mol_type = genomic DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaaggcggtgaatgagctgtttacttcttccaatcaaatcgggttaaaaacagatgtgacggattatcatattgatcaagtatccaatttagttgagtgtttatctgatgaattttgtctggatgaaaaaaaagaattgtccgagaaagtcaaacatgcgaagcgacttagtgatgagcggaatttacttcaagatccaaactttagagggatcaatagacaactagaccgtggctggagaggaagtacggatattaccatccaaggaggcgatgacgtattcaaagagaattacgttacgctattgggtacctttgatgagtgctatccaacgtatttatatcaaaaaatagatgagtcgaaattaaaagcctatacccgttaccaattaagagggtatatcgaagatagtcaagacttagaaatctatttaattcgctacaatgccaaacacgaaacagtaaatgtgccaggtacgggttccttatggccgctttcagccccaagtccaatcggaaaatgtgcccatcattcccatcatttctccttggacattgatgttggatgtacagacttaaatgaggacttaggtgtatgggtgatattcaagattaagacgcaagatggccatgcaagactaggaaatctagaatttctcgaagagaaaccattagtaggagaagcactagctcgtgtgaaaagagcggagaaaaaatggagagacaaacgtgaaaaattggaatgggaaacaaatattgtttataaagaggcaaaagaatctgtagatgctttatttgtaaactctcaatatgatagattacaagcggataccaacatcgcgatgattcatgcggcagataaacgcgttcatagcattcgagaagcttatctgcctgagctgtctgtgattccgggtgtcaatgcggctatttttgaagaattagaagggcgtattttcactgcattctccctatatgatgcgagaaatgtcattaaaaatggtgattttaataatggcttatcctgctggaacgtgaaagggcatgtagatgtagaagaacaaaacaaccaccgttcggtccttgttgttccggaatgggaagcagaagtgtcacaagaagttcgtgtctgtccgggtcgtggctatatccttcgtgtcacagcgtacaaggagggatatggagaaggttgcgtaaccattcatgagatcgagaacaatacagacgaactgaagtttagcaactgtgtagaagaggaagtatatccaaacaacacggtaacgtgtaatgattatactgcgactcaagaagaatatgagggtacgtacacttctcgtaatcgaggatatgacggagcctatgaaagcaattcttctgtaccagctgattatgcatcagcctatgaagaaaaagcatatacagatggacgaagagacaatccttgtgaatctaacagaggatatggggattacacaccactaccagctggctatgtgacaaaagaattagagtacttcccagaaaccgataaggtatggattgagatcggagaaacggaaggaacattcatcgtggacagcgtggaattacttcttatggaggaataa

SEQ ID NO: 14 - DNA, 3690 bp, Bacillus thuringiensis
  source 1..3690: mol_type = genomic DNA
atgacttcaaataggaaaaatgagaatgaaattataaatgctttatcgattccaacggtatcgaatccttccacgcaaatgaatctatcaccagatgctcgtattgaagatagcttgtgtgtagccgaggtgaacaatattgatccatttgttagcgcatcaacagtccaaacgggtataaacatagctggtagaatattgggcgtattaggtgtgccgtttgctggacaactagctagtttttatagttttcttgttggggaattatggcctagtggcagagatccatgggaaattttcctggaacatgtagaacaacttataagacaacaagtaacagaaaatactaggaatacggctattgctcgattagaaggtctaggaagaggctatagatcttaccagcaggctcttgaaacttggttagataaccgaaatgatgcaagatcaagaagcattattcttgagcgctatgttgctttagaacttgacattactactgctataccgcttttcagaatacgaaatgaagaagttccattattaatggtatatgctcaagctgcaaatttacacctattattattgagagacgcatccctttttggtagtgaatgggggatggcatcttccgatgttaaccaatattaccaagaacaaatcagatatacagaggaatattctaaccattgcgtacaatggtataatacagggctaaataacttaagagggacaaatgctgaaagttggttgcggtataatcaattccgtagagacctaacgttaggggtattagatttagtagccctattcccaagctatgatactcgcacttatccaatcaatacgagtgctcagttaacaagagaaatttatacagatccaattgggagaacaaatgcaccttcaggatttgcaagtacgaattggtttaataataatgcaccatcgttttctgccatagaggctgccattttcaggcctccgcatctacttgattttccagaacaacttacaatttacagtgcatcaagccgttggagtagcactcaacatatgaattattgggtgggacataggcttaacttccgcccaataggagggacattaaatacctcaacacaaggacttactaataatacttcaattaatcctgtaacattacagtttacgtctcgagacgtttatagaacagaatcaaatgcagggacaaatatactatttactactcctgtgaatggagtaccttgggctagatttaattttataaaccctcagaatatttatgaaagaggcgccactacctacagtcaaccgtatcagggagttgggattcaattatttgattcagaaactgaattaccaccagaaacaacagaacgaccaaattatgaatcatatagtcatagattatctcatataggactaatcataggaaacactttgagagcaccagtctattcttggacgcatcgtagtgcagatcgtacgaatacgattggaccaaatagaattacacaaataccattggtaaaagcactgaatcttcattcaggtgttactgttgttggagggccaggatttacaggtggggatatccttcgtagaacaaatacgggtacatttggagatatacgattaaatattaatgtgccattatcccaaagatatcgcgtaaggattcgttatgcttctactacagatttacaatttttcacgagaattaatggaaccactgttaatattggtaatttctcaagaactatgaataggggggataatttagaatatagaagttttagaactgcaggatttagtactccttttaattttttaaatgcccaaagcacattcacattgggtgctcagagtttttcaaatcaggaagtttatatagatagagtcgaatttgttccagcagaggtaacatttgaggcagaatatgatttagaaagagcacaaaaggcggtgaatgctctgtttacttctacaaatccaagaagattgaaaacagatgtgacagattatcatattgaccaagtgtccaatatggtggcatgtttatcagatgaattttgcttggatgagaagcgagaattatttgagaaagtgaaatatgcgaagcgactcagtgatgaaagaaacttactccaagatccaaacttcacattcatcagtgggcaattaagtttcgcatccatcgatggacaatcaaacttcccctctattaatgagctatctgaacatggatggtggggaagtgcgaatgttaccattcaggaagggaatgacgtatttaaagagaattacgtcacactaccgggtacttttaatgagtgttatccaaattatttatatcaaaaaataggagagtcagaattaaaagcttatacgcgctatcaattaagagggtatattgaagatagtcaagatctagagatttatttaattcgttacaatgcaaagcatgaaacattggatgttccaggtaccgattccctatggccgctttcagttgaaagcccaatcggaaggtgcggagaaccaaatcgatgcgcaccacattttgaatggaatcctgatctagattgttcctgcagagatggagaaagatgtgcgcatcattcccatcatttcactttggatattgatgttgggtgcacagacttgcatgagaacctaggcgtgtgggtggtattcaagattaagacgcaggaaggttatgcaagattaggaaatctggaatttatcgaagagaaaccattaattggagaagcactgtctcgtgtgaagagagcggaaaaaaaatggagagacaaacgggaaaaactacaattggaaacaaaacgagtatatacagaggcaaaagaagctgtggatgctttattcgtagattctcaatatgatcaattacaagcggatacaaacattggcatgattcatgcggcagataaacttgttcatcgaattcgagaggcgtatctttcagaattacctgttatcccaggtgtaaatgcggaaatttttgaagaattagaaggtcacattatcactgcaatgtccttatacgatgcgagaaatgtcgttaaaaatggtgattttaataatggattaacatgttggaatgtaaaagggcatgtagatgtacaacagagccatcatcgttctgaccttgttatcccagaatgggaagcagaagtgtcacaagcagttcgcgtctgtccggggcgtggctatatccttcgtgtcacagcgtacaaagagggatatggagagggctgcgtaacgatccatgaaatcgagaacaatacagacgaactaaaatttaaaaactgtgaagaagaggaagtgtatccaacggatacaggaacgtgtaatgattatactgcacaccaaggtacagcagcatgtaattcccgtaatgctggatatgaggatgcatatgaagttgatactacagcatctgttaattacaaaccgacttatgaagaagaaacgtatacagatgtacgaagagataatcattgtgaatatgacagagggtatgtgaattatccaccagtaccagctggttatgtgacaaaagaattagaatacttcccagaaacagatacagtatggattgagattggagaaacggaaggaaagtttattgtagatagcgtggaactactcctcatggaagaatag

SEQ ID NO: 15 - DNA, 20 bp, synthetic construct
  misc_feature 1..20: note = pET8NdeI-cry1Aa forward primer
  source 1..20: mol_type = other DNA
attccgccgcaggataacag

SEQ ID NO: 16 - DNA, 19 bp, synthetic construct
  misc_feature 1..19: note = pET8NdeI-cry1Ab forward primer
  source 1..19: mol_type = other DNA
tcagtaacagcagcgtgag

SEQ ID NO: 17 - DNA, 22 bp, synthetic construct
  misc_feature 1..22: note = pET8NdeI-cry1Ac forward primer
  source 1..22: mol_type = other DNA
atcccgccgcaaaacaacaacg

SEQ ID NO: 18 - DNA, 20 bp, synthetic construct
  misc_feature 1..20: note = pET8NdeI-cry1Ca forward primer
  source 1..20: mol_type = other DNA
caaccctgaccaataccatc

SEQ ID NO: 19 - DNA, 19 bp, synthetic construct
  misc_feature 1..19: note = pET8NdeI-cry1Da forward primer
  source 1..19: mol_type = other DNA
gcatctatcgcaagagtgg

SEQ ID NO: 20 - DNA, 24 bp, synthetic construct
  misc_feature 1..24: note = pET8NdeI-cry1Ea forward primer
  source 1..24: mol_type = other DNA
ttgacggtgaaaacagcctggtgg

SEQ ID NO: 21 - DNA, 20 bp, synthetic construct
  misc_feature 1..20: note = pET8NdeI-cry1Fa forward primer
  source 1..20: mol_type = other DNA
tgacagcctggacgaaatcc

SEQ ID NO: 22 - DNA, 20 bp, synthetic construct
  misc_feature 1..20: note = pET8NdeI-cry1Bb forward primer
  source 1..20: mol_type = other DNA
tcgcaccaataccatcggtc

SEQ ID NO: 23 - DNA, 18 bp, synthetic construct
  misc_feature 1..18: note = pET8NdeI-cry2Aa forward primer
  source 1..18: mol_type = other DNA
tatgccgccaacgaaaac

SEQ ID NO: 24 - DNA, 21 bp, synthetic construct
  misc_feature 1..21: note = pET8NdeI-cry9Ca forward primer
  source 1..21: mol_type = other DNA
ggacctgaacaataccatcac

SEQ ID NO: 25 - DNA, 19 bp, synthetic construct
  misc_feature 1..19: note = pET8NdeI-cry9Ea1 forward primer
  source 1..19: mol_type = other DNA
acatctgctgctgctgaag

SEQ ID NO: 26 - DNA, 19 bp, synthetic construct
  misc_feature 1..19: note = pET8NdeI-generic reverse primer
  source 1..19: mol_type = other DNA
gctagttattgctcagcgg

SEQ ID NO: 27 - DNA, 1311 bp, synthetic construct
  misc_feature 1..1311: note = 35S EF1 intron promoter
  source 1..1311: mol_type = other DNA
ctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacaggctcatttccacacccaccttcctcgtcttctccattcgcttcgccagcgctcgagcggccgaagggtttttggttttggtggccgaggcatcaggaggtgcgtcttccgtttcgatttcggattcgtcgatttgttttcgtgcttcgtgttgattgacatggctgaatcgcgcggtggattgtgttcttttgtttgtttccgcaatgtatgatggatcggatgatcttgtttggatgtctgcctcgtgcttttgtttgggattctggctcgtggaaacgagcgctcgttttctcttgctttgtcgtcgaatcggttgaatctgaggatagcgttttagatctgtgtagtgattggatctgctccgaacgagttgttgattggaccgaccggtttcgagatgatcaatcgaaatcatttttaagcgctgaggtgattgtgagatgaatattatatggtacctacaggattgaagttaatttggtatagtcaaaacgattttcaactgcaattttgtttgggtatttcgatgacttaaatttatcatgaggttcttgttcttgtttggggttaggtttcttccttgaggaaattttgtttctggtgtctaaagccaaaaaaaaaaaaaaaaacaaaaacaaaaaacctcgaatttctgctcctttatctgtctttctagtatgatcgagtacatgggtgcatggaatgatttgtggcttcttttggcttttattttggcagttgtggttcttaaagaatatggactgaattttcgttgggtgccaacatgagtagcttcagaaactaattctctgaatgaatgttttgtgtgctagcaatcgatcggtgaaattctattgttgcaattttttgttatgataaatcacttttcttaagtacgagaatcattttatcctggacagaatcaagccttataattcaag

SEQ ID NO: 28 - DNA, 26 bp, synthetic construct
  misc_feature 1..26: note = cry2Aa forward primer
  source 1..26: mol_type = other DNA
atgaacaacgtcttgaactccggtag

SEQ ID NO: 29 - DNA, 27 bp, synthetic construct
  misc_feature 1..27: note = cry1Ab forward primer
  source 1..27: mol_type = other DNA
atggataacaaccctaatatcaatgag

SEQ ID NO: 30 - DNA, 24 bp, synthetic construct
  misc_feature 1..24: note = cry1Bb forward primer
  source 1..24: mol_type = other DNA
atgaccagcaacaggaagaatgag

SEQ ID NO: 31 - DNA, 26 bp, synthetic construct
  misc_feature 1..26: note = RbcS forward primer
  source 1..26: mol_type = other DNA
gcaagaacccctttttcaaccctatg

SEQ ID NO: 32 - DNA, 26 bp, synthetic construct
  misc_feature 1..26: note = cry2Aa reverse primer
  source 1..26: mol_type = other DNA
tcagtacaatggtggcaagttggttg

SEQ ID NO: 33 - DNA, 24 bp, synthetic construct
  misc_feature 1..24: note = cry1Ab reverse primer
  source 1..24: mol_type = other DNA
tcacttttgggccctctcgagatc

SEQ ID NO: 34 - DNA, 24 bp, synthetic construct
  misc_feature 1..24: note = cry1Bb reverse primer
  source 1..24: mol_type = other DNA
tcacttttgggccctctcgagatc

SEQ ID NO: 35 - DNA, 30 bp, synthetic construct
  misc_feature 1..30: note = RbcS reverse primer
  source 1..30: mol_type = other DNA
gaatttctaaaaatgacaagtcaacaaatc

SEQ ID NO: 36 - DNA, 1200 bp, synthetic construct
  misc_feature 1..1200: note = Eucgr.J01502 promoter
  source 1..1200: mol_type = other DNA
caaatattacaacaataaggagtaaacttcactttttgagaatgaagatttttatacagagttaaaaatattacatcttttcagtaatatctaaagtaacatgtaaaaaattgactctatgtaaatgaggaaattaattgtatctataacttcacaaaagattgactctgtaatgtgcggcatgtgatgtggcattagatagagtaacaagaaagaagttaactttgtttagtaaaatataacacacttttggttttataaagaataatacatcttattttccttaattaggaaaaaaagaaaaggatgtatagtcaaagtcgatcaatattattagtatttcacttgacattgcatgtaccgctcgcttgttatttaggggaaaaaaaaagggtaccatactcttgttatttttccttttttcgcgaggatggtcgcgtttggttgggctttgggcctatccctacccaactccacgacatcaaatgcaacccctgcaaatttctttccccatcccttccccatttgcccatattatttgcacatgtaatggaaatatctctcttcctaaatttaacaccacatcttacgtttcacgagtcaattttttattagagcttttacaccatttttttcattaatgatttcgggatatgagaagaacccttctctaaattcttctcccccccccccccccccccacccaaaaaaaataaaaataaaattgggggacattttctcatgttaaaggtccacaaaaagagaaataaaaataaaatccgattgaatacatgattgaattattataattctagggttgatgactacatgtgattgaaattgacagatggggcatcaatggaatggcttagatccctcaattcctcaacaccagtgcttacaaaggagtggggatcagctcgagagacttcaacttcaccagtcaccaccatcacacattcatcgtcctccctaggatgagataagatcgccgagagcccgccacgtggcgctgtcatagcactggtaacgataaggctagatcaataaccttccccccttatgtggccagcaagcggtaatgtcatcacctatcagaaccgacggccaggatccgcagcctgtcaacgccggggatttgtggccctaggatacgtgaagggggaccttgtgtttatatataggatgaatcccacagtg

SEQ ID NO: 37 - protein, 623 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIASDSITQIPAVKGNFLFNGSVISGPGFTGGDLVRLNSSGNNIQNRGYIEVPIHFPSTSTRYRVRVRYASVTPIHLNVNWGNSSIFSNTVPATATSLDNLQSSDFGYFESANAFTSSLGNIVGVRNFSGTAGVIIDRFEFIPVTATLEAEYNLERAQK

SEQ ID NO: 38 - DNA, 1872 bp, synthetic construct
  misc_feature 1..1872: note = cry1Ac coding sequence optimized
  source 1..1872: mol_type = other DNA
atggataacaaccctaatatcaatgagtgcatcccctacaattgcctcagcaatcctgaggtggaggtgctcgggggtgagaggatagagactgggtacacccctatcgacatcagcctcagcctcacccagtttctcctctctgagttcgttcctggggccgggtttgtgctcgggctcgtggatataatctgggggatcttcggtcctagccagtgggacgccttccttgtgcagatcgagcaacttatcaaccagaggatcgaggagtttgctaggaaccaggccatcagcaggctcgaggggctcagcaatctctaccaaatctacgccgagagctttagggagtgggaggctgatcccactaaccctgccctcagggaggagatgcgtattcaattcaatgacatgaacagcgccctcactactgccatccctctcttcgccgtgcaaaattaccaagtgcccctcctcagcgtgtacgttcaagccgccaatcttcacctcagcgtgcttagggatgtgagcgtgttcgggcaaaggtgggggttcgatgccgccactatcaacagcaggtacaatgacctcaccaggctcatcgggaattacactgattacgctgtcagatggtacaacaccggtttggagagagtttggggtccagactccagagactgggttcgttacaaccagttccgtcgtgagttgaccttgactgtcttggacatcgtcgccttgttcccaaactacgactccagaagataccctatcaggactgtgagccaactcaccagggagatctacaccaatcctgtgctcgagaatttcgacgggagctttagggggagcgcccaagggatcgagagatctattagatctccacatttgatggatattttgaactccatcaccatctacaccgacgctcatagaggttactactactggagcgggcaccaaatcatggccagccctgtggggtttagcgggcctgagtttacctttcctctctacgggactatggggaatgccgcccctcaacaaaggatcgtggcccaactcgggcaaggggtgtacaggactctcagcagcaccctctacagaaggcctttcaatatcgggatcaacaatcaacaactcagcgtgctcgacggtactgagtttgcctacgggactagcagcaatctccctagcgccgtgtacaggaagagcggtactgtggacagcctcgatgagatcccccctcagaataacaacgtgcctcctaggcaagggtttagccacaggctcagccacgtgagcatgttcaggagcgggtttagcaatagcagcgtgagcatcatcagggcccctatgtttagctggattcataggagcgccgagttcaataacatcatcgcttctgactccattactcagatcccagccgtcaagggtaacttcttgttcaacggttccgttatctctggtcctggtttcactggtggtgacttggtccgtttgaactcctccggtaacaacatccagaaccgtggttacatcgaggtcccaattcacttcccatccacctccactagataccgtgtcagagtcagatacgcctctgtcaccccaattcatttgaacgttaattggggtaactcttccatcttctccaacaccgtccctgctaccgccacttctttggacaacttgcagtcctccgacttcggttacttcgagtctgctaacgccttcacctcctccttgggtaacatcgtcggtgtcagaaacttctccggaactgccggtgtcatcatcgacagattcgagttcatcccagtcactgccactttggaggccgagtacaacttggagcgtgctcaaaagtga

SEQ ID NO: 39 - protein, 630 aa, Bacillus thuringiensis
MEENNQNQCIPYNCLSNPEEVLLDGERISTGNSSIDISLSLVQFLVSNFVPGGGFLVGLIDFVWGIVGPSQWDAFLVQIEQLINERIAEFARNAAIANLEGLGNNFNIYVEAFKEWEEDPNNPETRTRVIDRFRILDGLLERDIPSFRISGFEVPLLSVYAQAANLHLAILRDSVIFGERWGLTTINVNENYNRLIRHIDEYADHCANTYNRGLNNLPKSTYQDWITYNRLRRDLTLTVLDIAAFFPNYDNRRYPIQPVGQLTREVYTDPLINFNPQLQSVAQLPTFNVMESSRIRNPHLFDILNNLTIFTDWFSVGRNFYWGGHRVISSLIGGGNITSPIYGREANQEPPRSFTFNGPVFRTLSNPTLRLLQQPWPAPPFNLRGVEGVEFSTPTNSFTYRGRGTVDSLTELPPEDNSVPPREGYSHRLCHATFVQRSGTPFLTTGVVFSWTDRSATLTNTIDPERINQIPLVKGFRVWGGTSVITGPGFTGGDILRRNTFGDFVSLQVNINSPITQRYRLRFRYASSRDARVIVLTGAASTGVGGQVSVNMPLQKTMEIGENLTSRTFRYTDFSNPFSFRANPDIIGISEQPLFGAGSISSGELYIDKIEIILADATFEAESDLERAQK

SEQ ID NO: 40 - DNA, 1893 bp, synthetic construct
  misc_feature 1..1893: note = cry1Ca coding sequence optimized
  source 1..1893: mol_type = other DNA
atggaggaaaacaaccaaaaccaatgtatcccttacaactgtttgtccaaccctgaggaggttcttcttgacggtgagcgtatttctactggtaattcttccatcgacatttccttgtctttggtccagtttttggtctctaacttcgtccctggtggaggtttcttggttggtcttattgacttcgtttggggtattgtcggaccatctcagtgggacgcctttttggtccagattgagcagcttatcaacgagagaatcgccgagtttgccagaaacgctgctatcgccaaccttgagggattgggtaacaatttcaacatttatgtcgaggccttcaaggagtgggaagaagatcctaacaatcctgagaccagaactcgtgtcatcgacagatttagaatcttggacggtttgttggagagagacatcccatccttccgtatctccggttttgaggtccctttgttgtctgtctacgcccaggccgccaacttgcacttggctattttgagagactccgttattttcggagagcgttggggtcttactaccatcaatgttaatgagaattacaacagattgattagacacattgatgaatacgctgatcattgtgctaatacttacaaccgtggattgaacaacttgcctaagtccacctaccaggactggattacttataatagattgcgtagagatttgaccttgaccgtccttgacatcgccgcttttttcccaaattacgataacagaagataccctatccaacctgtcggtcagttgaccagagaggtctacaccgacccattgattaacttcaacccacaattgcagtctgtcgcccagcttccaaccttcaacgtcatggaatcctcccgtatcagaaaccctcacttgtttgatattttgaacaatttgactatctttactgattggttctctgttggtagaaacttctattggggaggacaccgtgtcatttcctcccttatcggtggtggtaacatcacctcccctatctacggtcgtgaagctaaccaggagcctcctcgttccttcacttttaacggtccagtctttagaaccttgtctaaccctaccttgcgtttgttgcagcagccatggccagctccaccattcaacttgcgtggtgttgagggagttgagttctccactcctaccaactccttcacctatcgtggtagaggtaccgtcgactccttgaccgagcttcctcctgaagataattccgtcccacctcgtgagggttactcccacagattgtgccacgccacctttgttcaacgttccggtaccccattcttgactactggtgtcgttttctcttggaccgatcgttctgccactttgaccaacaccattgatcctgaaagaattaaccagattccacttgttaaaggtttccgtgtctggggtggaacttccgttatcactggtccaggtttcaccggaggtgatatcttgagaagaaacaccttcggtgatttcgtctccttgcaggtcaacatcaactcccctatcacccagagataccgtttgagattccgttatgcctcctctcgtgacgctagagtcatcgttttgactggtgctgcctccactggagttggaggtcaggtttccgtcaacatgccattgcagaagaccatggaaattggtgaaaaccttacttccagaactttcagatacactgacttttctaacccattctccttcagagctaatccagacattatcggtatctctgagcagccattgttcggtgctggatctatctcctctggtgaattgtacattgataagatcgaaattatcttggctgatgccacctttgaagccgaatccgatttggagagagcccagaaatga

SEQ ID NO: 41 - protein, 1155 aa, Bacillus thuringiensis
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAVNELFTSSNQIGLKTDVTDYHIDQVSNLVECLSDEFCLDEKKELSEKVKHAKRLSDERNLLQDPNFRGINRQLDRGWRGSTDITIQGGDDVFKENYVTLLGTFDECYPTYLYQKIDESKLKAYTRYQLRGYIEDSQDLEIYLIRYNAKHETVNVPGTGSLWPLSAPSPIGKCAHHSHHFSLDIDVGCTDLNEDLGVWVIFKIKTQDGHARLGNLEFLEEKPLVGEALARVKRAEKKWRDKREKLEWETNIVYKEAKESVDALFVNSQYDRLQADTNIAMIHAADKRVHSIREAYLPELSVIPGVNAAIFEELEGRIFTAFSLYDARNVIKNGDFNNGLSCWNVKGHVDVEEQNNHRSVLVVPEWEAEVSQEVRVCPGRGYILRVTAYKEGYGEGCVTIHEIENNTDELKFSNCVEEEVYPNNTVTCNDYTATQEEYEGTYTSRNRGYDGAYESNSSVPADYASAYEEKAYTDGRRDNPCESNRGYGDYTPLPAGYVTKELEYFPETDKVWIEIGETEGTFIVDSVELLLMEE

SEQ ID NO: 42 - DNA, 1866 bp, synthetic construct
  misc_feature 1..1866: note = cry1Ab truncated native
  source 1..1866: mol_type = other DNA
atggataacaatccgaacatcaatgaatgcattccttataattgtttaagtaaccctgaagtagaagtattaggtggagaaagaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctctttttgcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagatcatgctgtacgctggtacaatacgggattagagcgtgtatggggaccggattctagagattggataagatataatcaatttagaagagaattaacactaactgtattagatatcgtttctctatttccgaactatgatagtagaacgtatccaattcgaacagtttcccaattaacaagagaaatttatacaaacccagtattagaaaattttgatggtagttttcgaggctcggctcagggcatagaaggaagtattaggagtccacatttgatggatatacttaacagtataaccatctatacggatgctcatagaggagaatattattggtcagggcatcaaataatggcttctcctgtagggttttcggggccagaattcacttttccgctatatggaactatgggaaatgcagctccacaacaacgtattgttgctcaactaggtcagggcgtgtatagaacattatcgtccactttatatagaagaccttttaatatagggataaataatcaacaactatctgttcttgacgggacagaatttgcttatggaacctcctcaaatttgccatccgctgtatacagaaaaagcggaacggtagattcgctggatgaaataccgccacagaataacaacgtgccacctaggcaaggatttagtcatcgattaagccatgtttcaatgtttcgttcaggctttagtaatagtagtgtaagtataataagagctcctatgttctcttggatacatcgtagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctaccacaaatttacaattccatacatcaattgacggaagacctattaatcaggggaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaag

SEQ ID NO: 43 - protein, 1229 aa, Bacillus thuringiensis
MTSNRKNENEIINALSIPTVSNPSTQMNLSPDARIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQKAVNALFTSTNPRRLKTDVTDYHIDQVSNMVACLSDEFCLDEKRELFEKVKYAKRLSDERNLLQDPNFTFISGQLSFASIDGQSNFPSINELSEHGWWGSANVTIQEGNDVFKENYVTLPGTFNECYPNYLYQKIGESELKAYTRYQLRGYIEDSQDLEIYLIRYNAKHETLDVPGTDSLWPLSVESPIGRCGEPNRCAPHFEWNPDLDCSCRDGERCAHHSHHFTLDIDVGCTDLHENLGVWVVFKIKTQEGYARLGNLEFIEEKPLIGEALSRVKRAEKKWRDKREKLQLETKRVYTEAKEAVDALFVDSQYDQLQADTNIGMIHAADKLVHRIREAYLSELPVIPGVNAEIFEELEGHIITAMSLYDARNVVKNGDFNNGLTCWNVKGHVDVQQSHHRSDLVIPEWEAEVSQAVRVCPGRGYILRVTAYKEGYGEGCVTIHEIENNTDELKFKNCEEEEVYPTDTGTCNDYTAHQGTAACNSRNAGYEDAYEVDTTASVNYKPTYEEETYTDVRRDNHCEYDRGYVNYPPVPAGYVTKELEYFPETDTVWIEIGETEGKFIVDSVELLLMEE

SEQ ID NO: 44 - DNA, 1965 bp, synthetic construct
  misc_feature 1..1965: note = cry1Bb truncated native
  source 1..1965: mol_type = other DNA
atgacttcaaataggaaaaatgagaatgaaattataaatgctttatcgattccaacggtatcgaatccttccacgcaaatgaatctatcaccagatgctcgtattgaagatagcttgtgtgtagccgaggtgaacaatattgatccatttgttagcgcatcaacagtccaaacgggtataaacatagctggtagaatattgggcgtattaggtgtgccgtttgctggacaactagctagtttttatagttttcttgttggggaattatggcctagtggcagagatccatgggaaattttcctggaacatgtagaacaacttataagacaacaagtaacagaaaatactaggaatacggctattgctcgattagaaggtctaggaagaggctatagatcttaccagcaggctcttgaaacttggttagataaccgaaatgatgcaagatcaagaagcattattcttgagcgctatgttgctttagaacttgacattactactgctataccgcttttcagaatacgaaatgaagaagttccattattaatggtatatgctcaagctgcaaatttacacctattattattgagagacgcatccctttttggtagtgaatgggggatggcatcttccgatgttaaccaatattaccaagaacaaatcagatatacagaggaatattctaaccattgcgtacaatggtataatacagggctaaataacttaagagggacaaatgctgaaagttggttgcggtataatcaattccgtagagacctaacgttaggggtattagatttagtagccctattcccaagctatgatactcgcacttatccaatcaatacgagtgctcagttaacaagagaaatttatacagatccaattgggagaacaaatgcaccttcaggatttgcaagtacgaattggtttaataataatgcaccatcgttttctgccatagaggctgccattttcaggcctccgcatctacttgattttccagaacaacttacaatttacagtgcatcaagccgttggagtagcactcaacatatgaattattgggtgggacataggcttaacttccgcccaataggagggacattaaatacctcaacacaaggacttactaataatacttcaattaatcctgtaacattacagtttacgtctcgagacgtttatagaacagaatcaaatgcagggacaaatatactatttactactcctgtgaatggagtaccttgggctagatttaattttataaaccctcagaatatttatgaaagaggcgccactacctacagtcaaccgtatcagggagttgggattcaattatttgattcagaaactgaattaccaccagaaacaacagaacgaccaaattatgaatcatatagtcatagattatctcatataggactaatcataggaaacactttgagagcaccagtctattcttggacgcatcgtagtgcagatcgtacgaatacgattggaccaaatagaattacacaaataccattggtaaaagcactgaatcttcattcaggtgttactgttgttggagggccaggatttacaggtggggatatccttcgtagaacaaatacgggtacatttggagatatacgattaaatattaatgtgccattatcccaaagatatcgcgtaaggattcgttatgcttctactacagatttacaatttttcacgagaattaatggaaccactgttaatattggtaatttctcaagaactatgaataggggggataatttagaatatagaagttttagaactgcaggatttagtactccttttaattttttaaatgcccaaagcacattcacattgggtgctcagagtttttcaaatcaggaagtttatatagatagagtcgaatttgttccagcagaggtaacatttgaggcagaatatgatttagaaagagcacaaaag

SEQ ID NO: 45 - protein, 621 aa, synthetic construct
MDNNPNINECIPYNCLSNPEVEVLGGEQETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQK

SEQ ID NO: 46 - protein, 655 aa, synthetic construct
MTSNQKNENEIINALSIPTVSNPSTQMNLSPDAQIEDSLCVAEVNNIDPFVSASTVQTGINIAGRILGVLGVPFAGQLASFYSFLVGELWPSGRDPWEIFLEHVEQLIRQQVTENTRNTAIARLEGLGRGYRSYQQALETWLDNRNDARSRSIILERYVALELDITTAIPLFRIRNEEVPLLMVYAQAANLHLLLLRDASLFGSEWGMASSDVNQYYQEQIRYTEEYSNHCVQWYNTGLNNLRGTNAESWLRYNQFRRDLTLGVLDLVALFPSYDTRTYPINTSAQLTREIYTDPIGRTNAPSGFASTNWFNNNAPSFSAIEAAIFRPPHLLDFPEQLTIYSASSRWSSTQHMNYWVGHRLNFRPIGGTLNTSTQGLTNNTSINPVTLQFTSRDVYRTESNAGTNILFTTPVNGVPWARFNFINPQNIYERGATTYSQPYQGVGIQLFDSETELPPETTERPNYESYSHRLSHIGLIIGNTLRAPVYSWTHRSADRTNTIGPNRITQIPLVKALNLHSGVTVVGGPGFTGGDILRRTNTGTFGDIRLNINVPLSQRYRVRIRYASTTDLQFFTRINGTTVNIGNFSRTMNRGDNLEYRSFRTAGFSTPFNFLNAQSTFTLGAQSFSNQEVYIDRVEFVPAEVTFEAEYDLERAQK

SEQ ID NO: 47 - protein, 639 aa, synthetic construct
MNNVLNSGRTTICDAYNVVAHDPFSFEHKSLDTIQKEWMEWKRTDHSLYVAPVVGTVSSFLLKKVGSLIGKRILSELWGIIFPSGSTNLMQDILRETEQFLNQRLNTDTLARVNAELIGLQANIREFNQQVDNFLNPTQNPVPLSITSSVNTMQQLFLNRLPQFQIQGYQLLLLPLFAQAANMHLSFIRDVILNADEWGISAATLRTYRDYLRNYTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANENGTMIHLAPEDYTGFTISPIHATQVNNQTRTFISEKFGNQGDSLRFEQSNTTARYTLRGNGNSYNLYLRVSSIGNSTIRVTINGRVYTVSNVNTTTNNDGVNDNGARFSDINIGNIVASDNTNVTLDINVTLNSGTPFDLMNIMFVPTNLPPLYHHHHHH

SEQ ID NO: 48 - DNA, 15845 bp, synthetic construct
  source 1..15845: mol_type = other DNA
attttatatatatatgtgtgtctgctctttccactgatcagtttaaacttttggatttgactttctgacataatatcacgctcttaatttgatttgcatatgagaggcaatggttcaatatactctttatatttatatagtctcccttcatctattgacacacctttttggatttgactttctattgtgcggaacggatggtgaataatctcggaaccaaaatcaataaaagaacaattattattatgacttgaaacgtttgtgttgtcgtctagacgctagccaacaaagacccaccatccgtcgttgtcaatcacaagagaggaaagacaaagaaaacaagaaaaagattccataaagcattagccacgcattgcttattatagcactgaaaggctgccattcttgtaagttagattcttttgcattcagtcgcataatgcttccacatttgttatttctaagtgtcaagtttttcgtcattaactgtctttaacacggagcttaattgatgtattgacaagtgatcttttatggtgttatttattacatatttcttaagaggactgtcgtctttaggttttaagaagctaggacacaatgaattagatggaacatagacaaacttgccacaaagcactgacgtgatatgtaaaatcgtgacatgcattcaatgttcataaggcagttctatggtgggaatttactaaaagatgaatagtttaattagtctcccttttcttttccgtgggttgtcataaaaatgtattttgaactaatttgtacgcatgatattgagtctaactcttgagcgtattattctaaggaggtctagagattctttgacatgtcaagcatgaaagcatagactactttttacaactgcaaactgtggttggaagttcctcaagatttataagcttgtgattccagctttccccgtttaaagtcgtcttggcttgtgaaattgcaggatatcaatacaatggttcgttaatctatgcccgtgaaattgtaaatgacctaagcctatggtcccttagagtatcttaactcctatacgtgcgtttttacaaattacttagggtctatagattacgtagatcaaaatcatctcaactatcaatcatataattcaactataagatccacatgatcattaatcaggatgaactcaatgaacaatatggtctaaagatcatttaaagaaattcaatggaccgagagtctatcatgtaatccatgaccagtttcaaatcatatctatgcttgtgggttctttagatttcgtggatcatccaactcctgttcaagacactgttccaacaagataaggatgccttttccttctcagcaatatttcaaagttcagagttgtcaaaatcgcagtttcttgtcccttcaagtgagcaggagtcagctgctctccaactttctgtcgaattttccagcccaccatcttactaaatttgtattaatttcatcaatgattgacacccctgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttatcacttttgggccctctcgagatcgtactcggcctcgaaggtcacctcggcaggcacaaactcgattctgtcgatgtacacctcgttcccgctgttaaacacgtgggcgctgagagtgaacacgctgctcccgttgctgaaattgaaaggggtggtgaaccccacagtcctaaagctcccgctttggagattgctcccgctgctcatggtggcgctaaaattcccctggttgataggcctcccatcgatgctggtgtggaactggagattagtggtgctggcgtacctgatcctcactctgtacctctgagagagaggggcagtgatattcaccctgagggtgctgatttgcccagggctagtcctcctgaggatatcccccccagtaaacccaggccccttcaccacagaagtcccgctcccgagattggtgctcttggtgagagggatctgagtgatttggctgctagggatgatgttattgaactcggcgctcctatgaatccagctaaacataggggccctgatgatgctcacgctgctattgctaaacccgctcctgaacatgctcacgtggctgagcctgtggctaaacccttgcctaggaggcacgttgttattctgaggggggatctcatcgaggctgtccacagtaccgctcttcctgtacacggcgctagggagattgctgctagtcccgtaggcaaactcagtaccgtcgagcacgctgagttgttgattgttgatcccgatattgaaaggccttctgtagagggtgctgctgagagtcctgtacaccccttgcccgagttgggccacgatcctttgttgaggggcggcattccccatagtcccgtagagaggaaaggtaaactcaggcccgctaaaccccacagggctggccatgatttggtgcccgctccagtagtactcccccctgtgggcgtcggtgtagatggtgatgctgttgaggatgtccatgaggtgagggctcctgatgctcccctcgatcccttgggcgctccccctaaagctcccgtcgaaattctcgagcacaggattggtgtagatctccctggtgagttggctcacagtcctgatagggtaagtcctgctgtcgtaattagggaagaggctcacgatgtcgagcacagtgagagttaattccctcctgaactggttgtacctgatccaatccctgctgtcaggcccccacaccctctcgagcccagtattgtaccacctcacggcgtgatcagtgtaattcccgatgagcctggtgaggtcattgtacctgctgttgatagtggcggcatcgaacccccacctttgcccgaacacgctcacatccctaagcacgctgaggtgaagattggcggcttgaacgtacacgctgaggaggggcacttggtaattttgcacggcgaagagagggatggcagtagtgagggcgctgttcatgtcattgaattgaatacgcatctcctccctgagggcagggttagtgggatcagcctcccactccctaaagctctcggcgtagatttggtagagattgctgagcccctcgagcctgctgatggcctggttcctagcaaactcctcgatcctctggttgataagttgctcgatctgcacaaggaaggcgtcccactggctaggaccgaagatcccccagattatatccacgagcccgagcacaaacccggccccaggaacgaactcagagaggagaaactgggtgaggctgaggctgatgtcgataggggtgtacccagtctctatcctctcacccccgagcacctccacctcaggattgctgaggcaattgtaggggatgcactcattgatattagggttgttatccatcccggggatcctctagagtcgactgtaattgtaaatagtaattgtaatgttgtttgttgtttgttgttgttggtaattgttgaaggatctcaagaagcctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtaggagccaccttccttttctactgtcctttcgatgaagtgacagatagctgggcaatggaatccgaggaggtttcccgatattaccctttgttgaaaagtctcaatagccctctggtcttctgaggggggatcgatccccatcccgcaagaggcccggcagtaccggcataaccaagctatgcctaagcatccagggtgacggtgccgaggatgacgatgagcgcattgttagatttcatacacggtgcctgactgcgttagcaatttaactgtgataaactaccgcattaaagcttgaattcgcgatcgcactccacttgatacttcatttataagtgaaaaggctttaaaggcagctgatgttgctcgagtcgattgtcgagctcaacgagatctgacactcaacctactcatgctcggttggaatcgaggcgagttctagttcgcaactaagcaatcacttttactcttacgtctaactctagagattcttcaaggtactctaccgtcgctcagatgtttcccttcttctcctccatttgaagcttggctcaatcacttcattatgtaagctcatgaatggctagcatccattgcaattccttcaacatctagcttggaaggattaacgaagtcatctttcgataaccaaaagaaacagtactcgtcgtggacacacccggcatcatcaagcaatttgcactcagtggaggacatcaacaacgaaagtaagcaagcaagcaaacagctgaattcattcatagaataaatcgggtcttaatgcaacgcatctgcgaactcaaaagtacacaaatatcacaggacttgggtaataagggaaaaatggatcattcgacatctagctacaattgaaaggtttggaacgatgccgcttcgagggtaaccccacgatccaccatgacataagcttgcggcgcgccgagctcttatcacttttgggccctctcgagatcgtactcagcctcaaaggtcacctcggcaggcacgaactccactctatcgatgtacacttcctgattgctgaagctctgagccccgagagtaaaggtgctttgagcattgaggaaattaaaaggagtgctaaacccagcggtcctgaagctcctgtactcgagattgtcccctctgttcatggtcctgctgaaattcccgatattcacggtagtcccattgattctggtgaaaaactggagatcggtagtgctggcgtatcttatcctcaccctgtacctctggctgagaggcacattgatattgagcctgatgtccccaaaggtaccggtgttagtcctcctgaggatatcccccccagtaaacccaggcccaccaacaacagtcaccccgctatgaagattgagggccttaacaagggggatctgggtgattctattgggcccgatggtattggtcctgtcggcgctcctatgagtccagctatacacaggggctctgagagtgttcccgatgataagcccgatatggctgagcctatggctatagctctcgtagttgggcctctcggtggtctcgggaggaagttcggtctcgctatcaaagagttgaatacccacaccttggtagggttgagagtaagtggtggcccccctctcatagatgttctgagggttgatgaagttgaatctggcccaaggcaccccgttcacaggggtagtgaagaggatattggtcccggcgttgctctcagtcctgtacacgtccctagaagtgaattggagagtcacggggttgatgctagtattgttggtgagcccttgggtgctagtgttgagggtccccccgataggcctaaaattaagcctgtgccccacccagtaattcatgtgttgagtgctgctccacctgctgctggcgctgtagatagtgagctgctcaggaaagtcgagaaggtgagggggcctgaatatggcggcctctatagcgctaaagctgggagcattgttattgaaccaattggtgctggcgaaaccgctgggggcattagtcctacctataggatcagtgtagatctccctagtgagctgggcgctagtattgatggggtaggttctagtgtcgtagctaggaaagagggccacgaggtcaagcaccccaagagtgagatccctcctgaattggttgtacctaagccagctttcggcgttggtccccctgagattgttaagcccggtattgtaccattgcacgcagtggttgctgtattcctcggtgtacctgatttgctcctggtagtactggttcacgtcagagctggccataccccactcagacccaaaaagagaggcatctctgaggagaaggaggtggagattggcagcctgggcgtaaaccataaggaggggcacctcctcgttcctgatcctaaaaagggggatggcggtggttatgtcaagctcgagggccacgtatctctcgaggatgatgctcctgctcctagcgtcatttctattgtcgagccaggtctcgagggcttgttggtagctcctgtaccccctcccgagcccttcaagtctggcgatagcggtattcctggtattttcggtcacctgttgccttatgagctgttcaacgtgctcaagaaagatctcccaaggatcacgaccgctgggccataattccccaacgaggaagctatagaagctggcaagctgaccggcaaaaggcaccccaagcacccctaaaatacgcccagcgatgttgatcccggtctgcacggtgctggcgctaacaaaagggtcgatattattaacttcggccacacagaggctatcctctatcctagcgtcagggctgagattcatctgagtagaggggttgctaacagtggggatgctaagggcgttgattatctcgttctcattcttcctgttgctggtcatcccgggatcccaatacgcgcctcttctccctcttcttcttcttcttcttcctttggtgagtgagaacttgtatgctctcttatggaacttccactgtgggattcatcctatatataaacacaaggtcccccttcacgtatcctagggccacaaatccccggcgttgacaggctgcggatcctggccgtcggttctgataggtgatgacattaccgcttgctggccacataaggggggaaggttattgatctagccttatcgttaccagtgctatgacagcgccacgtggcgggctctcggcgatcttatctcatcctagggaggacgatgaatgtgtgatggtggtgactggtgaagttgaagtctctcgagctgatccccactcctttgtaagcactggtgttgaggaattgagggatctaagccattccattgatgccccatctgtcaatttcaatcacatgtagtcatcaaccctagaattataataattcaatcatgtattcaatcggattttatttttatttctctttttgtggacctttaacatgagaaaatgtcccccaattttatttttattttttttgggtgggggggggggggggggggagaagaatttagagaagggttcttctcatatcccgaaatcattaatgaaaaaaatggtgtaaaagctctaataaaaaattgactcgtgaaacgtaagatgtggtgttaaatttaggaagagagatatttccattacatgtgcaaataatatgggcaaatggggaagggatggggaaagaaatttgcaggggttgcatttgatgtcgtggagttgggtagggataggcccaaagcccaaccaaacgcgaccatcctcgcgaaaaaaggaaaaataacaagagtatggtaccctttttttttcccctaaataacaagcgagcggtacatgcaatgtcaagtgaaatactaataatattgatcgactttgactatacatccttttctttttttcctaattaaggaaaataagatgtattattctttataaaaccaaaagtgtgttatattttactaaacaaagttaacttctttcttgttactctatctaatgccacatcacatgccgcacattacagagtcaatcttttgtgaagttatagatacaattaatttcctcatttacatagagtcaattttttacatgttactttagatattactgaaaagatgtaatatttttaactctgtataaaaatcttcattctcaaaaagtgaagtttactccttattgttgtaatatttggcgatcgccctgcaggcatgcaagcttgaattcgtccactttatcatatttaaaccaaacctacgggggagtcaacgtaaccaaccctgccttagcatcttttccctaacggcctcctgcctaagcggtacttctagcttcgaacggcgtctgggctccaggtttagtcgtctcatgtctggtttatattcacgacaaagatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaaatagagagagatagatttgtagagagagactggtgatttcaccggttcagaagaactcatcgagcagtcggtagaatgcgatacgctgtgaatcaggtgcagcaatcccatagaggacaaggaaacgatctgcccattcacctccaagttcctcagcgatgtcacgagttgcaagtgcaatgtcctggtacctatctgcaactccgagtctaccacagtcgatgaatccagagaacctcccattctcaaccatgatgttgggcaagcaagcatctccgtgagtcacaacaagatcctctccatctggcatcctagctttgagccttgcgaaaagttcagctggagcaagtccttgatgctcctcatcaagatcgtcttggtccaccaaacctgcttccattctagtcctagccctctcaatcctatgcttagcctggtgatcgaaaggacatgtggcaggatcaagtgtgtgaagtcttcgcatggcatcagccattatagacaccttctcagctggtgccagatgactagaaagcaagtcctgtcctggcacttcaccaagaagcaaccaatcccttccagcctctgtaacaacgtcaagaactgcagcgcaaggaacaccagttgtagcaagccatgaaagtctggcagcctcatcctgaagctcattcaaagctccggacaagtcagtcttgacgaagagaacaggtctcccttgtgcactaagcctgaaaactgcagcatcagagcaacctatggtctgttgtgcccagtcatacccaaagagtcgttcaacccatgcagctggagatccagcatgaagaccatcctgttcaatcatcctaggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattgtaccactagtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggccggccgcaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcagccgggcggccgaccggtgagctcttatcagtacaatggtggcaagttggttgggacgaacatgatgttcatcaagtcaaatggggtaccagaatttaaggtgacgttgatgtcaagggtgacattggtattgtcggaggcaacaatgttaccgatgttgatgtcagagaaacgggcaccgttatcattgactccgtcgttgttggtggtagtgttgacgttggagacagtgtaaacacgtccgttgatggtgacacggatagtggagttaccgatggaggaaactctcaagtacaagttataagagttaccgtttcctctcaaggtgtatctggcggtggtgttagattgctcgaaacgcaaggagtctccttggttaccaaacttttcagaaataaaagttctagtttgattgttaacttgggtggcgtgaataggggagatggtaaaaccggtgtaatcctctggggcaaggtgaatcatggttccgttctcgttggcagcgtagatgttgttctttctgttgtggacagaaaccaagtaggctctggcacctcctggggtaccggatggggattcgatatttctgatctggttgtaatgcaatggtctggtcaagtcctcgttacggatgacaagtgggacaccagaaatgtttctaatgaaataatctggaaagtaattagaattacctctggcagagaaggcaccacaacgcaaggacaaggtggtttgaaaggactcggtctgccagttggtagaggtagcaacaccctctctgtcagttccagagtccaaccaggaacggacgaaaggggtggacaatggaggcaagacagtggagcagttgaagttatgattcaagttagtggcaccaatcaaaccagaagagacaccaccggagtagttaacacgagcagagttaagggagtgagtggtagtggaacctggaagacctccgatgtttgggaaggtgatagaaagtctggtaccggagataccggacaaaatatagttagagttaacttgaaaaagggagtacaagaatggccaattttgggcggtaaaggactgagtctgttgaggaccagaaccagaggcgtaaaggttggctccagaggagaccatcaaagactgatacttaaacaaggaccaaatagaaacgtattcaaaaacgttcaagaacatgtaggtacggaactccaacatgtcgtgcaaacgggtgttcaaacctctaaaggcagtctgataagtgttgatgcagtagttggagtagtctctagtgtagttacgcaagtagtcacggtaggttctcaaggtagcagcagagattccccattcgtcggcgttaaggatgacgtctctgataaaggacaagtgcatgttggcggcttgggcaaacaaaggcaacaacaacaactgatatccctggatctgaaactgtggcaatctgttcaaaaacaactgctgcatggtgttgacggaggaggtgatggacaatgggactgggttctgggtagggttcaaaaaattgtcaacctgttggttgaactctctgatgttggcctgcaaaccaatcaattcggcgttgacacgggccaaagtatcggtgttcaatctctgattaaggaattgctcagtttctctcaaaatatcctgcatcaaattggtggaaccggatgggaagataataccccacaactcggacaaaattctcttaccgatcaaggaaccaaccttcttcaacaagaaagaagagacagtaccaacgacaggagcgacgtacaaagagtggtcggttctcttccactccatccactctttctggatggtgtccaaggacttatgttcaaaagaaaaagggtcgtgggcgacgacattataggcgtcgcagatggtggttctaccggagttcaagacgttgttcatcccgggattatgcttgaattataaggcttgattctgtccaggataaaatgattctcgtacttaagaaaagtgatttatcataacaaaaaattgcaacaatagaatttcaccgatcgattgctagcacacaaaacattcattcagagaattagtttctgaagctactcatgttggcacccaacgaaaattcagtccatattctttaagaaccacaactgccaaaataaaagccaaaagaagccacaaatcattccatgcacccatgtactcgatcatactagaaagacagataaaggagcagaaattcgaggttttttgtttttgtttttttttttttttttggctttagacaccagaaacaaaatttcctcaaggaagaaacctaaccccaaacaagaacaagaacctcatgataaatttaagtcatcgaaatacccaaacaaaattgcagttgaaaatcgttttgactataccaaattaacttcaatcctgtaggtaccatataatattcatctcacaatcacctcagcgcttaaaaatgatttcgattgatcatctcgaaaccggtcggtccaatcaacaactcgttcggagcagatccaatcactacacagatctaaaacgctatcctcagattcaaccgattcgacgacaaagcaagagaaaacgagcgctcgtttccacgagccagaatcccaaacaaaagcacgaggcagacatccaaacaagatcatccgatccatcatacattgcggaaacaaacaaaagaacacaatccaccgcgcgattcagccatgtcaatcaacacgaagcacgaaaacaaatcgacgaatccgaaatcgaaacggaagacgcacctcctgatgcctcggccaccaaaaccaaaaacccttcggccgctcgagcgctggcgaagcgaatggagaagacgaggaaggtgggtgtggaaatgagcctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtaggagccaccttccttttctactgtcctttcgatgaagtgacagatagctgggcaatggaatccgaggaggtttcccgatattaccctttgttgaaaagtctcaatagccctctggtcttctgagtatcgatccccatcccgcaagaggcccggcagtaccggcataaccaagctatgcctaagcatccagggtgacggtgccgaggatgacgatgagcgcattgttagatttcatacacggtgcctgactgcgttagcaatttaactgtgataaactaccgcattaaagcttcggccggccgtaaaactatcagtgtttgacacgcaagtgtctccacacgtttatggttagaatctatttggtgatcacttattcctgaaaacaagaaaaaaaagagaaaaaataattttttttttttttgcttcttgattctattttaaatttattttcaagaacaaatctattctcaggagtagaataattagctgatattaccaaattgacttcatttttttagaactaaagagcagatatagccaataattgaccaattactaaataggctcttagcatactttttttttttggttctttatgtagtcacgtctattgtcaaccttgcaaatagaaccatcaacaagtaaaataatattttttttttcttcttcggttattcacatgtgatgcgagacccaccacaatttcttgaaatgtgactgaacttggatttagtagggtaatatctttattggggaatgccgtgtcccttgcttcacatggaaacaaatctcgtgattacgaaaggatgcatcttgcccgcataaccccacacaaatcttcctctgttcttttctccttctcctcctcctccttccccacatgttatattagagaacgacaatttcaattcaatgttatctcaccagctatgggcatctcaggctcagagaccccatcacaagcctcaccaccgacaccgaagccaacagcaccaccaccgccaacaacgacaaattcgcctccttcgattgaagtggagactgcaaactgtgcttcctgtgggttcacagaggaatgcacccctgcatatatcctccggattcgacaacggtaccaagggagatggctctgtgggctctgcattcaggcggtgaaggacgagctcttgcgatcggacaggctcatatccaccgaggaggcattgaataggcacatcagcttttgcaagaagttcaagtcatccgatcccttgaaggagacagagaaccccatctctgccatggggaaactgatcaggcgcagcttggattcgccgagggctatccggacaaaatcgagcagcgctttgccagggatcgatgcagtaacgggcataactacgccggtgcttgctcggtcagggagctgcttctcttctatatctagttgaatgaggaataagtatatgttcatggagcagatgaactaggagcagataaaatatgtttctggcatgggcatctgggttttctatgcttttccttttattcaaattactggatgatgaactggatttggctatgcagatgacaattttctatgtacatatgatcccggatctgatgtattagttaggtatgctcatgaagtcacgtcgatgattttatgttgctacatacttcttgcttggacatttcatagtctaagtcgataaagaaatcttctgcaaaagagtgttcgtatccatgtccaagggtccatgtccatgtgctttcagtttcaattgatgaaaatgtcgagaaaacccattagcaatccaccaagcaacgtccatatagtttct

SEQ ID NO: 49 - DNA, 81 bp, synthetic construct
  source 1..81: mol_type = other DNA
ttttccagcccaccatcttactaaatttgtattaatttcatcaatgattgacacccctgtactggggtggtttttcttttc

SEQ ID NO: 50 - DNA, 51 bp, synthetic construct
  source 1..51: mol_type = other DNA
ggccggccgtaaaactatcagtgtttgacacgcaagtgtctccacacgttt

SEQ ID NO: 51 - DNA, 160 bp, synthetic construct
  source 1..160: mol_type = other DNA
tcgcagtttcttgtcccttcaagtgagcaggagtcagctgctctccaactttctgtcgaattttccagcccaccatcttactaaatttgtattaatttcatcaatgattgacacccctgtactggggtggtttttcttttcaccagtgagacgggcaaca

SEQ ID NO: 52 - DNA, 114 bp, synthetic construct
  source 1..114: mol_type = other DNA
accgcattaaagcttcggccggccgtaaaactatcagtgtttgacacgcaagtgtctccacacgtttatggttagaatctatttggtgatcacttattcctgaaaacaagaaaa

SEQ ID NO: 53 - DNA, 255 bp, synthetic construct
  source 1..255: mol_type = other DNA
atgccttttccttctcagcaatatttcaaagttcagagttgtcaaaatcgcagtttcttgtcccttcaagtgagcaggagtcagctgctctccaactttctgtcgaattttccagcccaccatcttactaaatttgtattaatttcatcaatgattgacacccctgtactggggtggtttttcttttcaccagtgagacgggcaacagctgattgcccttcaccgcctggccctgagagagttgcagcaagcggt

SEQ ID NO: 54 - DNA, 493 bp, synthetic construct
  source 1..493: mol_type = other DNA
gatgacgatgagcgcattgttagatttcatacacggtgcctgactgcgttagcaatttaactgtgataaactaccgcattaaagcttcggccggccgtaaaactatcagtgtttgacacgcaagtgtctccacacgtttatggttagaatctatttggtgatcacttattcctgaaaacaagaaaaaaaagagaaaaaataattttttttttttttgcttcttgattctattttaaatttattttcaagaacaaatctattctcaggagtagaataattagctgatattaccaaattgacttcatttttttagaactaaagagcagatatagccaataattgaccaattactaaataggctcttagcatactttttttttttggttctttatgtagtcacgtctattgtcaaccttgcaaatagaaccatcaacaagtaaaataatattttttttttcttcttcggttattcacatgtgatgcgagacccacc

SEQ ID NO: 55 - DNA, 1500 bp, Eucalyptus grandis
  source 1..1500: mol_type = genomic DNA
attttatatatatatgtgtgtctgctctttccactgatcagtttaaacttttggatttgactttctgacataatatcacgctcttaatttgatttgcatatgagaggcaatggttcaatatactctttatatttatatagtctcccttcatctattgacacacctttttggatttgactttctattgtgcggaacggatggtgaataatctcggaaccaaaatcaataaaagaacaattattattatgacttgaaacgtttgtgttgtcgtctagacgctagccaacaaagacccaccatccgtcgttgtcaatcacaagagaggaaagacaaagaaaacaagaaaaagattccataaagcattagccacgcattgcttattatagcactgaaaggctgccattcttgtaagttagattcttttgcattcagtcgcataatgcttccacatttgttatttctaagtgtcaagtttttcgtcattaactgtctttaacacggagcttaattgatgtattgacaagtgatcttttatggtgttatttattacatatttcttaagaggactgtcgtctttaggttttaagaagctaggacacaatgaattagatggaacatagacaaacttgccacaaagcactgacgtgatatgtaaaatcgtgacatgcattcaatgttcataaggcagttctatggtgggaatttactaaaagatgaatagtttaattagtctcccttttcttttccgtgggttgtcataaaaatgtattttgaactaatttgtacgcatgatattgagtctaactcttgagcgtattattctaaggaggtctagagattctttgacatgtcaagcatgaaagcatagactactttttacaactgcaaactgtggttggaagttcctcaagatttataagcttgtgattccagctttccccgtttaaagtcgtcttggcttgtgaaattgcaggatatcaatacaatggttcgttaatctatgcccgtgaaattgtaaatgacctaagcctatggtcccttagagtatcttaactcctatacgtgcgtttttacaaattacttagggtctatagattacgtagatcaaaatcatctcaactatcaatcatataattcaactataagatccacatgatcattaatcaggatgaactcaatgaacaatatggtctaaagatcatttaaagaaattcaatggaccgagagtctatcatgtaatccatgaccagtttcaaatcatatctatgcttgtgggttctttagatttcgtggatcatccaactcctgttcaagacactgttccaacaagataaggatgccttttccttctcagcaatatttcaaagttcagagttgtcaaaatcgcagtttcttgtcccttcaagtgagcaggagtcagctgctctccaactttctgtcgaattttccagcccaccatcttactaaatttgtattaatttcatcaatgattgacacccc

SEQ ID NO: 56 - DNA, 1500 bp, Eucalyptus grandis
  source 1..1500: mol_type = genomic DNA
cgcaagtgtctccacacgtttatggttagaatctatttggtgatcacttattcctgaaaacaagaaaaaaaagagaaaaaataattttttttttttttgcttcttgattctattttaaatttattttcaagaacaaatctattctcaggagtagaataattagctgatattaccaaattgacttcatttttttagaactaaagagcagatatagccaataattgaccaattactaaataggctcttagcatactttttttttttggttctttatgtagtcacgtctattgtcaaccttgcaaatagaaccatcaacaagtaaaataatattttttttttcttcttcggttattcacatgtgatgcgagacccaccacaatttcttgaaatgtgactgaacttggatttagtagggtaatatctttattggggaatgccgtgtcccttgcttcacatggaaacaaatctcgtgattacgaaaggatgcatcttgcccgcataaccccacacaaatcttcctctgttcttttctccttctcctcctcctccttccccacatgttatattagagaacgacaatttcaattcaatgttatctcaccagctatgggcatctcaggctcagagaccccatcacaagcctcaccaccgacaccgaagccaacagcaccaccaccgccaacaacgacaaattcgcctccttcgattgaagtggagactgcaaactgtgcttcctgtgggttcacagaggaatgcacccctgcatatatcctccggattcgacaacggtaccaagggagatggctctgtgggctctgcattcaggcggtgaaggacgagctcttgcgatcggacaggctcatatccaccgaggaggcattgaataggcacatcagcttttgcaagaagttcaagtcatccgatcccttgaaggagacagagaaccccatctctgccatggggaaactgatcaggcgcagcttggattcgccgagggctatccggacaaaatcgagcagcgctttgccagggatcgatgcagtaacgggcataactacgccggtgcttgctcggtcagggagctgcttctcttctatatctagttgaatgaggaataagtatatgttcatggagcagatgaactaggagcagataaaatatgtttctggcatgggcatctgggttttctatgcttttccttttattcaaattactggatgatgaactggatttggctatgcagatgacaattttctatgtacatatgatcccggatctgatgtattagttaggtatgctcatgaagtcacgtcgatgattttatgttgctacatacttcttgcttggacatttcatagtctaagtcgataaagaaatcttctgcaaaagagtgttcgtatccatgtccaagggtccatgtccatgtgctttcagtttcaattgatgaaaatgtcgagaaaacccattagcaatccaccaagcaacgtccatatagtttct

SEQ ID NO: 57 - DNA, 12845 bp, synthetic construct
  source 1..12845: mol_type = other DNA
tgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcttatcacttttgggccctctcgagatcgtactcggcctcgaaggtcacctcggcaggcacaaactcgattctgtcgatgtacacctcgttcccgctgttaaacacgtgggcgctgagagtgaacacgctgctcccgttgctgaaattgaaaggggtggtgaaccccacagtcctaaagctcccgctttggagattgctcccgctgctcatggtggcgctaaaattcccctggttgataggcctcccatcgatgctggtgtggaactggagattagtggtgctggcgtacctgatcctcactctgtacctctgagagagaggggcagtgatattcaccctgagggtgctgatttgcccagggctagtcctcctgaggatatcccccccagtaaacccaggccccttcaccacagaagtcccgctcccgagattggtgctcttggtgagagggatctgagtgatttggctgctagggatgatgttattgaactcggcgctcctatgaatccagctaaacataggggccctgatgatgctcacgctgctattgctaaacccgctcctgaacatgctcacgtggctgagcctgtggctaaacccttgcctaggaggcacgttgttattctgaggggggatctcatcgaggctgtccacagtaccgctcttcctgtacacggcgctagggagattgctgctagtcccgtaggcaaactcagtaccgtcgagcacgctgagttgttgattgttgatcccgatattgaaaggccttctgtagagggtgctgctgagagtcctgtacaccccttgcccgagttgggccacgatcctttgttgaggggcggcattccccatagtcccgtagagaggaaaggtaaactcaggcccgctaaaccccacagggctggccatgatttggtgcccgctccagtagtactcccccctgtgggcgtcggtgtagatggtgatgctgttgaggatgtccatgaggtgagggctcctgatgctcccctcgatcccttgggcgctccccctaaagctcccgtcgaaattctcgagcacaggattggtgtagatctccctggtgagttggctcacagtcctgatagggtaagtcctgctgtcgtaattagggaagaggctcacgatgtcgagcacagtgagagttaattccctcctgaactggttgtacctgatccaatccctgctgtcaggcccccacaccctctcgagcccagtattgtaccacctcacggcgtgatcagtgtaattcccgatgagcctggtgaggtcattgtacctgctgttgatagtggcggcatcgaacccccacctttgcccgaacacgctcacatccctaagcacgctgaggtgaagattggcggcttgaacgtacacgctgaggaggggcacttggtaattttgcacggcgaagagagggatggcagtagtgagggcgctgttcatgtcattgaattgaatacgcatctcctccctgagggcagggttagtgggatcagcctcccactccctaaagctctcggcgtagatttggtagagattgctgagcccctcgagcctgctgatggcctggttcctagcaaactcctcgatcctctggttgataagttgctcgatctgcacaaggaaggcgtcccactggctaggaccgaagatcccccagattatatccacgagcccgagcacaaacccggccccaggaacgaactcagagaggagaaactgggtgaggctgaggctgatgtcgataggggtgtacccagtctctatcctctcacccccgagcacctccacctcaggattgctgaggcaattgtaggggatgcactcattgatattagggttgttatccatcccggggatcctctagagtcgactgtaattgtaaatagtaattgtaatgttgtttgttgtttgttgttgttggtaattgttgaaggatctcaagaagcctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtaggagccaccttccttttctactgtcctttcgatgaagtgacagatagctgggcaatggaatccgaggaggtttcccgatattaccctttgttgaaaagtctcaatagccctctggtcttctgaggggggatcgatccccatcccgcaagaggcccggcagtaccggcataaccaagctatgcctaagcatccagggtgacggtgccgaggatgacgatgagcgcattgttagatttcatacacggtgcctgactgcgttagcaatttaactgtgataaactaccgcattaaagcttgaattcgcgatcgcactccacttgatacttcatttataagtgaaaaggctttaaaggcagctgatgttgctcgagtcgattgtcgagctcaacgagatctgacactcaacctactcatgctcggttggaatcgaggcgagttctagttcgcaactaagcaatcacttttactcttacgtctaactctagagattcttcaaggtactctaccgtcgctcagatgtttcccttcttctcctccatttgaagcttggctcaatcacttcattatgtaagctcatgaatggctagcatccattgcaattccttcaacatctagcttggaaggattaacgaagtcatctttcgataaccaaaagaaacagtactcgtcgtggacacacccggcatcatcaagcaatttgcactcagtggaggacatcaacaacgaaagtaagcaagcaagcaaacagctgaattcattcatagaataaatcgggtcttaatgcaacgcatctgcgaactcaaaagtacacaaatatcacaggacttgggtaataagggaaaaatggatcattcgacatctagctacaattgaaaggtttggaacgatgccgcttcgagggtaaccccacgatccaccatgacataagcttgcggcgcgccgagctcttatcacttttgggccctctcgagatcgtactcagcctcaaaggtcacctcggcaggcacgaactccactctatcgatgtacacttcctgattgctgaagctctgagccccgagagtaaaggtgctttgagcattgaggaaattaaaaggagtgctaaacccagcggtcctgaagctcctgtactcgagattgtcccctctgttcatggtcctgctgaaattcccgatattcacggtagtcccattgattctggtgaaaaactggagatcggtagtgctggcgtatcttatcctcaccctgtacctctggctgagaggcacattgatattgagcctgatgtccccaaaggtaccggtgttagtcctcctgaggatatcccccccagtaaacccaggcccaccaacaacagtcaccccgctatgaagattgagggccttaacaagggggatctgggtgattctattgggcccgatggtattggtcctgtcggcgctcctatgagtccagctatacacaggggctctgagagtgttcccgatgataagcccgatatggctgagcctatggctatagctctcgtagttgggcctctcggtggtctcgggaggaagttcggtctcgctatcaaagagttgaatacccacaccttggtagggttgagagtaagtggtggcccccctctcatagatgttctgagggttgatgaagttgaatctggcccaaggcaccccgttcacaggggtagtgaagaggatattggtcccggcgttgctctcagtcctgtacacgtccctagaagtgaattggagagtcacggggttgatgctagtattgttggtgagcccttgggtgctagtgttgagggtccccccgataggcctaaaattaagcctgtgccccacccagtaattcatgtgttgagtgctgctccacctgctgctggcgctgtagatagtgagctgctcaggaaagtcgagaaggtgagggggcctgaatatggcggcctctatagcgctaaagctgggagcattgttattgaaccaattggtgctggcgaaaccgctgggggcattagtcctacctataggatcagtgtagatctccctagtgagctgggcgctagtattgatggggtaggttctagtgtcgtagctaggaaagagggccacgaggtcaagcaccccaagagtgagatccctcctgaattggttgtacctaagccagctttcggcgttggtccccctgagattgttaagcccggtattgtaccattgcacgcagtggttgctgtattcctcggtgtacctgatttgctcctggtagtactggttcacgtcagagctggccataccccactcagacccaaaaagagaggcatctctgaggagaaggaggtggagattggcagcctgggcgtaaaccataaggaggggcacctcctcgttcctgatcctaaaaagggggatggcggtggttatgtcaagctcgagggccacgtatctctcgaggatgatgctcctgctcctagcgtcatttctattgtcgagccaggtctcgagggcttgttggtagctcctgtaccccctcccgagcccttcaagtctggcgatagcggtattcctggtattttcggtcacctgttgccttatgagctgttcaacgtgctcaagaaagatctcccaaggatcacgaccgctgggccataattccccaacgaggaagctatagaagctggcaagctgaccggcaaaaggcaccccaagcacccctaaaatacgcccagcgatgttgatcccggtctgcacggtgctggcgctaacaaaagggtcgatattattaacttcggccacacagaggctatcctctatcctagcgtcagggctgagattcatctgagtagaggggttgctaacagtggggatgctaagggcgttgattatctcgttctcattcttcctgttgctggtcatcccgggatcccaatacgcgcctcttctccctcttcttcttcttcttcttcctttggtgagtgagaacttgtatgctctcttatggaacttccactgtgggattcatcctatatataaacacaaggtcccccttcacgtatcctagggccacaaatccccggcgttgacaggctgcggatcctggccgtcggttctgataggtgatgacattaccgcttgctggccacataaggggggaaggttattgatctagccttatcgttaccagtgctatgacagcgccacgtggcgggctctcggcgatcttatctcatcctagggaggacgatgaatgtgtgatggtggtgactggtgaagttgaagtctctcgagctgatccccactcctttgtaagcactggtgttgaggaattgagggatctaagccattccattgatgccccatctgtcaatttcaatcacatgtagtcatcaaccctagaattataataattcaatcatgtattcaatcggattttatttttatttctctttttgtggacctttaacatgagaaaatgtcccccaattttatttttattttttttgggtgggggggggggggggggggagaagaatttagagaagggttcttctcatatcccgaaatcattaatgaaaaaaatggtgtaaaagctctaataaaaaattgactcgtgaaacgtaagatgtggtgttaaatttaggaagagagatatttccattacatgtgcaaataatatgggcaaatggggaagggatggggaaagaaatttgcaggggttgcatttgatgtcgtggagttgggtagggataggcccaaagcccaaccaaacgcgaccatcctcgcgaaaaaaggaaaaataacaagagtatggtaccctttttttttcccctaaataacaagcgagcggtacatgcaatgtcaagtgaaatactaataatattgatcgactttgactatacatccttttctttttttcctaattaaggaaaataagatgtattattctttataaaaccaaaagtgtgttatattttactaaacaaagttaacttctttcttgttactctatctaatgccacatcacatgccgcacattacagagtcaatcttttgtgaagttatagatacaattaatttcctcatttacatagagtcaattttttacatgttactttagatattactgaaaagatgtaatatttttaactctgtataaaaatcttcattctcaaaaagtgaagtttactccttattgttgtaatatttggcgatcgccctgcaggcatgcaagcttgaattcgtccactttatcatatttaaaccaaacctacgggggagtcaacgtaaccaaccctgccttagcatcttttccctaacggcctcctgcctaagcggtacttctagcttcgaacggcgtctgggctccaggtttagtcgtctcatgtctggtttatattcacgacaaagatctatagggactttaggtgatctggattttagtactggattttggttttaggaattagaaattttattgatagaagtattttacaaatacaaatacatactaagggtttcttatatgctcaacacgtgagcgaaaccctataagaaccctaattcccttatctgggaactactcacacattattatggagaaaatagagagagatagatttgtagagagagactggtgatttcaccggttcagaagaactcatcgagcagtcggtagaatgcgatacgctgtgaatcaggtgcagcaatcccatagaggacaaggaaacgatctgcccattcacctccaagttcctcagcgatgtcacgagttgcaagtgcaatgtcctggtacctatctgcaactccgagtctaccacagtcgatgaatccagagaacctcccattctcaaccatgatgttgggcaagcaagcatctccgtgagtcacaacaagatcctctccatctggcatcctagctttgagccttgcgaaaagttcagctggagcaagtccttgatgctcctcatcaagatcgtcttggtccaccaaacctgcttccattctagtcctagccctctcaatcctatgcttagcctggtgatcgaaaggacatgtggcaggatcaagtgtgtgaagtcttcgcatggcatcagccattatagacaccttctcagctggtgccagatgactagaaagcaagtcctgtcctggcacttcaccaagaagcaaccaatcccttccagcctctgtaacaacgtcaagaactgcagcgcaaggaacaccagttgtagcaagccatgaaagtctggcagcctcatcctgaagctcattcaaagctccggacaagtcagtcttgacgaagagaacaggtctcccttgtgcactaagcctgaaaactgcagcatcagagcaacctatggtctgttgtgcccagtcatacccaaagagtcgttcaacccatgcagctggagatccagcatgaagaccatcctgttcaatcatcctaggctatcgttcgtaaatggtgaaaattttcagaaaattgcttttgctttaaaagaaatgatttaaattgctgcaatagaagtagaatgcttgattgcttgagattcgtttgttttgtatatgttgtgttgagaattgtaccactagtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggccggccgcaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcagccgggcggccgaccggtgagctcttatcagtacaatggtggcaagttggttgggacgaacatgatgttcatcaagtcaaatggggtaccagaatttaaggtgacgttgatgtcaagggtgacattggtattgtcggaggcaacaatgttaccgatgttgatgtcagagaaacgggcaccgttatcattgactccgtcgttgttggtggtagtgttgacgttggagacagtgtaaacacgtccgttgatggtgacacggatagtggagttaccgatggaggaaactctcaagtacaagttataagagttaccgtttcctctcaaggtgtatctggcggtggtgttagattgctcgaaacgcaaggagtctccttggttaccaaacttttcagaaataaaagttctagtttgattgttaacttgggtggcgtgaataggggagatggtaaaaccggtgtaatcctctggggcaaggtgaatcatggttccgttctcgttggcagcgtagatgttgttctttctgttgtggacagaaaccaagtaggctctggcacctcctggggtaccggatggggattcgatatttctgatctggttgtaatgcaatggtctggtcaagtcctcgttacggatgacaagtgggacaccagaaatgtttctaatgaaataatctggaaagtaattagaattacctctggcagagaaggcaccacaacgcaaggacaaggtggtttgaaaggactcggtctgccagttggtagaggtagcaacaccctctctgtcagttccagagtccaaccaggaacggacgaaaggggtggacaatggaggcaagacagtggagcagttgaagttatgattcaagttagtggcaccaatcaaaccagaagagacaccaccggagtagttaacacgagcagagttaagggagtgagtggtagtggaacctggaagacctccgatgtttgggaaggtgatagaaagtctggtaccggagataccggacaaaatatagttagagttaacttgaaaaagggagtacaagaatggccaattttgggcggtaaaggactgagtctgttgaggaccagaaccagaggcgtaaaggttggctccagaggagaccatcaaagactgatacttaaacaaggaccaaatagaaacgtattcaaaaacgttcaagaacatgtaggtacggaactccaacatgtcgtgcaaacgggtgttcaaacctctaaaggcagtctgataagtgttgatgcagtagttggagtagtctctagtgtagttacgcaagtagtcacggtaggttctcaaggtagcagcagagattccccattcgtcggcgttaaggatgacgtctctgataaaggacaagtgcatgttggcggcttgggcaaacaaaggcaacaacaacaactgatatccctggatctgaaactgtggcaatctgttcaaaaacaactgctgcatggtgttgacggaggaggtgatggacaatgggactgggttctgggtagggttcaaaaaattgtcaacctgttggttgaactctctgatgttggcctgcaaaccaatcaattcggcgttgacacgggccaaagtatcggtgttcaatctctgattaaggaattgctcagtttctctcaaaatatcctgcatcaaattggtggaaccggatgggaagataataccccacaactcggacaaaattctcttaccgatcaaggaaccaaccttcttcaacaagaaagaagagacagtaccaacgacaggagcgacgtacaaagagtggtcggttctcttccactccatccactctttctggatggtgtccaaggacttatgttcaaaagaaaaagggtcgtgggcgacgacattataggcgtcgcagatggtggttctaccggagttcaagacgttgttcatcccgggattatgcttgaattataaggcttgattctgtccaggataaaatgattctcgtacttaagaaaagtgatttatcataacaaaaaattgcaacaatagaatttcaccgatcgattgctagcacacaaaacattcattcagagaattagtttctgaagctactcatgttggcacccaacgaaaattcagtccatattctttaagaaccacaactgccaaaataaaagccaaaagaagccacaaatcattccatgcacccatgtactcgatcatactagaaagacagataaaggagcagaaattcgaggttttttgtttttgtttttttttttttttttggctttagacaccagaaacaaaatttcctcaaggaagaaacctaaccccaaacaagaacaagaacctcatgataaatttaagtcatcgaaatacccaaacaaaattgcagttgaaaatcgttttgactataccaaattaacttcaatcctgtaggtaccatataatattcatctcacaatcacctcagcgcttaaaaatgatttcgattgatcatctcgaaaccggtcggtccaatcaacaactcgttcggagcagatccaatcactacacagatctaaaacgctatcctcagattcaaccgattcgacgacaaagcaagagaaaacgagcgctcgtttccacgagccagaatcccaaacaaaagcacgaggcagacatccaaacaagatcatccgatccatcatacattgcggaaacaaacaaaagaacacaatccaccgcgcgattcagccatgtcaatcaacacgaagcacgaaaacaaatcgacgaatccgaaatcgaaacggaagacgcacctcctgatgcctcggccaccaaaaccaaaaacccttcggccgctcgagcgctggcgaagcgaatggagaagacgaggaaggtgggtgtggaaatgagcctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtaggagccaccttccttttctactgtcctttcgatgaagtgacagatagctgggcaatggaatccgaggaggtttcccgatattaccctttgttgaaaagtctcaatagccctctggtcttctgagtatcgatccccatcccgcaagaggcccggcagtaccggcataaccaagctatgcctaagcatccagggtgacggtgccgaggatgacgatgagcgcattgttagatttcatacacggtgcctgactgcgttagcaatttaactgtgataaactaccgcattaaagcttcggccggccgtaaaactatcagtgtttgaca

SEQ ID NO: 58 - DNA, 20 bp, synthetic construct
  source 1..20: mol_type = other DNA
ttttccagcccaccatctta

SEQ ID NO: 59 - DNA, 21 bp, synthetic construct
  source 1..21: mol_type = other DNA
gaaaagaaaaaccaccccagt

SEQ ID NO: 60 - DNA, 20 bp, synthetic construct
  source 1..20: mol_type = other DNA
ggccggccgtaaaactatca

SEQ ID NO: 61 - DNA, 20 bp, synthetic construct
  source 1..20: mol_type = other DNA
aaacgtgtggagacacttgc

SEQ ID NO: 62 - DNA, 20 bp, synthetic construct
  source 1..20: mol_type = other DNA
tcgcagtttcttgtcccttc

SEQ ID NO: 63 - DNA, 22 bp, synthetic construct
  source 1..22: mol_type = other DNA
tgttgcccgtctcactggtgaa

SEQ ID NO: 64 - DNA, 20 bp, synthetic construct
  source 1..20: mol_type = other DNA
accgcattaaagcttcggcc

SEQ ID NO: 65 - DNA, 25 bp, synthetic construct
  source 1..25: mol_type = other DNA
ttttcttgttttcaggaataagtga

SEQ ID NO: 66 - DNA, 20 bp, synthetic construct
  source 1..20: mol_type = other DNA
atgccttttccttctcagca

SEQ ID NO: 67 - DNA, 21 bp, synthetic construct
  source 1..21: mol_type = other DNA
accgcttgctgcaactctctc

SEQ ID NO: 68 - DNA, 20 bp, synthetic construct
  source 1..20: mol_type = other DNA
gatgacgatgagcgcattgt

SEQ ID NO: 69 - DNA, 19 bp, synthetic construct
  source 1..19: mol_type = other DNA
ggtgggtctcgcatcacat

(19) United States
(12) Patent Application Publication
(10) Pub. No.: US 2025/0388922 A1
(43) Pub. Date: Dec. 25, 2025

(54) A BACILLUS THURINGIENSIS PESTICIDAL PROTEIN (BT PP) COMBINATION USEFUL FOR PLANT PROTECTION

(71) Applicants: FuturaGene Israel, Ltd., Park Tamar, Rehovot (IL); SUZANO S.A., Pituba Salvador (BR)

(72) Inventors: Dror Avisar, Ramat Gan (IL); Shelly Azulay, Ness Ziona (IL); Sivan Livne, Rehovot (IL); Carolina Da Silva Rocha, Itapetininga (BR); Esteban Roberto Gonzalez, Itapetininga (BR); Murici Carlos Candelaria, Itapetininga (BR); Anselmo Anzevedo Dos Santos

(21) Appl. No.: 18/837,818

(22) PCT Filed: Feb. 15, 2023
(86) PCT No.: PCT/IB2023/051354

§ 371 Date: Aug. 12, 2024

Related U.S. Application Data
(60) Provisional application No. 63/310,518, filed on Feb. 15, 2022.

(51) Int. Cl. C12N 15/82; C07K 14/325; C12N 5/04; C12N 5/10; C12N 15/11; C12Q 1/6813; C12Q 1/6844; C12Q 1/6895

(52) U.S. Cl. CPC C12N 15/8286; C07K 14/325; C12N 5/04; C12N 5/10; C12N 15/11; C12N 15/8201; C12Q 1/6813; C12Q 1/6844; C12Q 1/6895; C12N 2510/00

(57) ABSTRACT

The present disclosure relates to the use of Bacillus thuringiensis (Bt) pesticidal proteins (PP), and, specifically, to the use of a combination of Bt PP for inhibiting, killing or managing insect pests. More specifically, the pesticidal protein (PP) combination may be co-expressed in a plant cell or plant. The present disclosure also encompasses methods for expressing an effective combination of two or 2 or 3 Bt PP in plants such as Eucalyptus and methods useful for managing insect pests using a combination of Bt PP. The disclosure also relates to Tg event No. 49, to a recombinant DNA molecule corresponding to the insertion locus (insert and flanking genomic sequences) in Tg event No. 49, and to plants comprising the recombinant DNA molecule. The invention also relates to uses of such plants to manage insect pest infestation, and to produce further plants comprising the recombinant DNA molecule.

### FIELD OF THE INVENTION

The present disclosure relates in general to compositions and methods useful to render plants resistant to pests. More specifically, the present disclosure relates to the field of transgenic plants harboring Bacillus thuringiensis Cry-family pesticidal proteins (PP), also known as Bt Toxins, for pest resistance, and more specifically, insect resistance.

### BACKGROUND OF THE INVENTION

Bacillus thuringiensis (Bt) bacterial strains have been long known for their pesticidal and specifically their insecticidal activity. Bt δ-endotoxins are pore-forming pesticidal proteins (PP, or toxins). They are naturally occurring and expressed in Bt and can be found in their crystalline matrix.

Bt spores are known to possess insecticidal activity when ingested by certain insects. To exert an insecticidal function, naturally occurring δ-endotoxins must first be ingested by the insect and undergo proteolytic activation (cleavage of an N-terminus, and sometimes of a C-terminal extension-portion) to form an active “Bt PP” insecticidal protein. Once the protein has been solubilized and processed, it has been reported that it may bind specific receptors on the surface of the insects' mid-gut epithelium and subsequently integrate into the lipid bilayer of the brush border membrane opening non-selective ion channels that disrupt the osmotic pressure and lead to cell collapse. These disruptive changes to the insect digestive tract prevent intact digestion leading to insect death.

Some Bt PP are effective against insects, innocuous to humans, vertebrates and plants and are completely biodegradable. They have been used effectively for the control of insect pests in agriculture by spraying formulated Bt reagents and later by expressing the insecticidal active proteins in transgenic crops. The first Bt-based transgenic technology, which was based on a single PP, led to relatively rapid development of insect resistance and thus the design of Bt transgenic plants that are less prone to the development of resistance by their target pest are of particular value.

### SUMMARY OF INVENTION

In one aspect, the present disclosure provides a Bacillus thuringiensis (Bt)-derived Pesticidal Protein (PP) combination expressed in a plant cell, and useful for inhibiting, killing or managing specific insect pests. As described herein, the combination can include a double-combination of any two of the Bacillus thuringiensis pesticidal proteins (Bt PP): (1) Cry2Aa (2) Cry1Ab and (3) Cry1Bb (Double Bt PP) or a triple-combination of the Bacillus thuringiensis pesticidal proteins (Bt PP): (1) Cry2Aa (2) Cry1Ab and (3) Cry1Bb (Triple Bt PP). In another aspect, the present disclosure provides a method useful for of inhibiting, killing or managing insect pests by transgenically co-expressing in a plant a combination of the above-mentioned Double Bt PP or Triple Bt PP. In another aspect, the present disclosure provides a nucleic acid construct that includes a Rubisco promoter sequence operably linked to a nucleic acid encoding a Bt PP. In another aspect, the present disclosure provides a nucleic acid construct that includes the Double Bt PP-combination or Triple Bt PP-combination of the above-mentioned Bt PP.

In another aspect the present disclosure provides a method of generating a transgenic plant, having steps of introducing into a plant cell one or more constructs with nucleic acid sequences encoding Cry1Bb, Cry2Aa and Cry1Ab, and regenerating the plant cell into an intact plant. The above method may include producing an intact plant in which said nucleic acid sequence/s encoding Bt PP are inserted in specific chromosomes, or in chromosomal locations, found to be particularly effective for Bt PP production and/or inhibiting, killing or managing specific insect pests. The method may also include the step of selecting for growing in the field, plants, with a given efficiency in inhibiting, killing or managing specific insect pests as determined by in vitro assay. Such methods may involve using the plants, or parts thereof, as a sole in vitro food source for insects, such as caterpillars, and determining pesticidal activity against said insects.

The invention is also directed to a particular beneficial plant referred to a Tg event No. 49, to a recombinant DNA molecule representing the insertion locus (insert and flanking genomic sequences) in Tg event No. 49, and to plants such as progeny plants and newly produced plants comprising the recombinant DNA molecule. The invention is also directed to uses of such plants to manage insect pest infestation, and to produce further plants comprising the recombinant DNA molecule.

In one aspect the invention provides a transgenic plant, comprising at least two of.
(i) a first nucleic acid sequence encoding a Cry2Aa Bacillus thuringiensis (Bt) pesticidal protein (PP) or an active portion thereof, and
(ii) a second nucleic acid sequence encoding a Cry1Ab Bt PP or an active portion thereof, and
(iii) a third nucleic acid sequence encoding a Cry1Bb Bt PP or an active portion thereof.

In one embodiment the transgenic plant comprises all three of:
(i) a first nucleic acid sequence encoding a Cry2Aa Bacillus thuringiensis (Bt) pesticidal protein (PP) or an active portion thereof, and
(ii) a second nucleic acid sequence encoding a Cry1Ab Bt PP or an active portion thereof, and
(iii) a third nucleic acid sequence encoding a Cry1Bb Bt PP or an active portion thereof.

In one embodiment:
said Cry2Aa Bt PP is characterized by comprising a sequence with at least 95% sequence identity to SEQ ID NO: 1,
said Cry1Ab Bt PP is characterized by comprising a sequence with at least 95% sequence identity to SEQ ID NO: 3, and
said Cry1Bb Bt PP is characterized by comprising a sequence with at least 95% sequence identity to SEQ ID NO: 5.

In a further embodiment:
said Cry2Aa Bt PP is characterized by comprising a sequence with at least 99% sequence identity to SEQ ID NO: 1,
said Cry1Ab Bt PP is characterized by comprising a sequence with at least 99% sequence identity to SEQ ID NO: 3, and
said Cry1Bb Bt PP is characterized by comprising a sequence with at least 99% sequence identity to SEQ ID NO: 5.

In a further embodiment:
said nucleic acid sequence encoding said Cry2Aa Bt PP is characterized by comprising a sequence with at least 95% sequence identity to SEQ ID NO: 2,
said nucleic acid sequence encoding said Cry1Ab Bt PP is characterized by comprising a sequence with at least 95% sequence identity to SEQ ID NO: 4, and
said nucleic acid sequence encoding said Cry1Bb Bt PP is characterized by comprising a sequence with at least 95% sequence identity to SEQ ID NO: 6

In a further embodiment:
said nucleic acid sequence encoding said Cry2Aa Bt PP is characterized by comprising a sequence with at least 99% sequence identity to SEQ ID NO: 2,
said nucleic acid sequence encoding said Cry1Ab Bt PP is characterized by comprising a sequence with at least 99% sequence identity to SEQ ID NO: 4, and
said nucleic acid sequence encoding said Cry1Bb Bt PP is characterized by comprising a sequence with at least 99% sequence identity to SEQ ID NO: 6

In a further embodiment:
said nucleic acid sequence encoding said Cry2Aa Bt PP comprises the sequence shown in SEQ ID NO: 2, and
said nucleic acid sequence encoding said Cry1Ab Bt PP comprises the sequence shown in SEQ ID NO: 4, and
said nucleic acid sequence encoding said Cry1Bb Bt PP comprises the sequence shown in SEQ ID NO: 6

In a further embodiment the transgenic plant comprises the nucleic acid sequence of a Rubisco promoter comprising a sequence with at least 90% sequence identity to the Rubisco promoter of SEQ ID NO: 36, operably linked to at least one of the first, second or third nucleic acid sequences.

In one embodiment the Rubisco promoter comprises the nucleic acid sequence shown in SEQ ID NO: 36. In a further embodiment said Rubisco promoter is operably linked to said nucleic acid sequence encoding said Cry1Bb Bt PP.

In a further embodiment said plant expresses at least two of the Cry2Aa, Cry1Ab, and Cry1Bb Bt PP. Ina further embodiment said plant expresses all three of the Cry2Aa, Cry1Ab, and Cry1Bb Bt PP.

In a further embodiment said expression of the at least two, or all three Bt PP confers increased resistance to insect pest infestation relative to that in a plant that does not express the at least two, or all least 3, Bt PP respectively.

In a further embodiment said combined expression of the at least two, or all three Bt PP respectively, produces synergistic effect on increasing resistance to insect pest infestation.

In one embodiment said each of the Bt PP binds to a different binding site in the gut membrane of an insect. In a further embodiment said each binding sites is in a different receptor in the gut membrane of the insect.

In one embodiment said plant is a Eucalyptus plant.

In a further aspect the invention provides seed from the transgenic plant of the invention, wherein said seed comprises said nucleic acid sequences encoding at least two, or all three, of the Cry2Aa, Cry1Ab, and Cry1Bb Bt PP.

In a further aspect the invention provides tissue or plant material from the transgenic plant of the invention, wherein said tissue or plant material comprises the nucleic acids sequences encoding the at least two of, or all three of, Cry2Aa, Cry1Ab, and Cry1Bb Bt PP.

In a further aspect the invention provides a method of inhibiting growth of, or killing, or managing an insect pest infestation of a plant, comprising transgenically co-expressing in said plant at least two, or all three, of Cry2Aa, Cry1Ab and Cry1Bb Bacillus thuringiensis-derived Pesticidal Proteins (Bt PP).

In a further aspect the invention provides a method for producing a plant that is resistant to insect pest infestation, the method comprising transforming the plant with nucleic acids encoding at least two, or all three of Cry2Aa, Cry1Ab and Cry1Bb Bacillus thuringiensis-derived Pesticidal Proteins (Bt PP).

In a further aspect the invention provides a method for producing a progeny plant that is resistant to insect pest infestation, the method comprising at least one of:
a) propagating a first plant of the invention to produce the progeny plant, and
b) crossing a first plant of the invention with second plant to produce the progeny plant,
wherein the progeny plant comprises the recited nucleic acids from the first plant as defined in above.

In a further aspect the invention provides a method of controlling insect pest infestation the method comprising growing the plant of the invention in the field.

In one embodiment said insect pest infestation is caused by an insect pest is selected from the group consisting of: Thyrinteina arnobia (Geometridae), Physocleora dukinfeldia (Geometridae), Sarsina violascens (Erebidae), Glena spp. (Geometridae), Melanolophia consmilaria (Geometridae), Eacles spp. (Saturniidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

In a further embodiment said insect pest is selected from the group consisting of: Thyrinteina arnobia (Geometridae), Thyrinteina leucocerae (Geometridae), Physocleora dukinfeldia (Geometridae), Sarsina violascens (Eribidae), Oxydia vesulia (Geometridae), Melanolophia consimilaria (Geometridae), and Spodoptera cosmioides (Noctuidae).

In a further embodiment said insect pest is Thyrinteina arnobia (Geometridae) or Physocleora dukinfeldia (Geometridae).

In various embodiments of the methods of the invention, said plant is a woody plant.

In various embodiments said plant is a Eucalyptus plant.

In various embodiments of the methods of the invention:
said Cry1Bb Bt PP is characterized by comprising a sequence with at least 95% sequence identity to SEQ ID NO: 5,
said Cry2Aa Bt PP is characterized by comprising a sequence with at least 95% sequence identity to SEQ ID NO: 1 and
said Cry1Ab Bt PP is characterized by comprising a sequence with at least 95% sequence identity to SEQ ID NO: 3.

In a further aspect the invention provides a nucleic acid construct, comprising:
(i) a first nucleic acid comprising the sequence of the Rubisco promoter of SEQ ID NO: 36 or a sequence having at least 80% sequence similarity to the Rubisco promoter of SEQ ID NO: 36,
(ii) a second nucleic acid sequence encoding at least one of the Bt PP selected from the group consisting of Cry1Bb, Cry1Ab and Cry2Aa,
wherein said first nucleic acid and said second nucleic acid are operably linked.

In one embodiment said Bt PP is characterized by comprising a sequence with at least 95% sequence identity to one of the amino acid sequences selected from the group consisting of SEQ ID NO: 5, SEQ ID NO: 3 or SEQ ID NO: 1.

In a further aspect the invention provides a nucleic acid construct, comprising at least two, or at least three, of
(i) a first nucleic acid sequence encoding Cry2Aa Bt PP,
(ii) a second nucleic acid sequence encoding Cry1Ab Bt PP, and
(iii) a third nucleic acid sequence encoding Cry1Bb Bt PP,
wherein expression of said first nucleic acid, said second nucleic acid and said third nucleic acid are directed by a promoter that is functional in a plant cell.

In one embodiment:
said first nucleic acid is characterized by comprising a sequence with at least 95% sequence identity to SEQ ID NO: 2,
said second nucleic acid is characterized by comprising a sequence with at least 95% sequence identity to SEQ ID NO: 4, and
said third nucleic acid is characterized by comprising a sequence with at least 95% sequence identity to SEQ ID NO: 6.

In a further embodiment:
a) said first nucleic acid comprises the sequence of SEQ ID NO: 2,
b) said second nucleic acid comprises the sequence of SEQ ID NO: 4, and
c) said third nucleic acid comprises the sequence of SEQ ID NO: 6.

In a further aspect the invention provides a method of making a transgenic plant, comprising:
a) introducing at least one nucleic acid construct of the invention into plant cells to produce transformed plant cells, and
b) culturing the transformed plant cells under conditions appropriate to regenerate a plant, thereby making a transgenic plant.

In one embodiment the method of further comprising at least one of:
a) screening for the presence of the said nucleic acid sequences in said transformed plant cells or plant,
b) screening said regenerated plant for insect resistance, and
c) selecting said plants cell or plant, on the basis of the screening in a) or b).

In a further aspect the invention provides a recombinant DNA molecule comprising a nucleotide sequence selected from the group consisting of SEQ ID NO:48, SEQ ID NO:49, SEQ ID NO:50, SEQ ID NO:51, SEQ ID NO:52, SEQ ID NO:53 and SEQ ID NO:54 and a complete complement thereof of any of the foregoing.

In one embodiment the recombinant DNA molecule is from eucalyptus Tg event No:49.

In a further aspect the invention provides a DNA molecule comprising a polynucleotide segment of sufficient length to function as a DNA probe that hybridizes specifically under stringent hybridization conditions with eucalyptus Tg event No:49 DNA in a sample, wherein detecting hybridization of said DNA molecule under said stringent hybridization conditions is diagnostic for the presence of eucalyptus Tg event No:49 DNA in said sample.

In one embodiment said sample comprises a plant, part thereof, tissue thereof or cell thereof, of or from eucalyptus Tg event No:49.

In a further aspect the invention provides a pair of DNA molecules, comprising a first DNA molecule and a second DNA molecule different from the first DNA molecule, that function as DNA primers when used together in an amplification reaction with a sample containing a plant, part thereof or tissue thereof, of or from eucalyptus Tg event No:49 template DNA to produce an amplicon diagnostic for the presence of said eucalyptus Tg event No:49 DNA in said sample, wherein said amplicon comprises the recombinant DNA molecule of the invention.

In one embodiment:
a) The first DNA molecule comprises the sequence of any one of SEQ ID NO: 58, SEQ ID NO: 60, SEQ ID NO: 62, SEQ ID NO: 64, SEQ ID NO: 66, SEQ ID NO: 68, and
b) The second DNA molecule comprises the sequence of any one of SEQ ID NO: 59, SEQ ID NO: 61, SEQ ID NO: 63, SEQ ID NO: 65, SEQ ID NO: 67, SEQ ID NO: 69.

In a further aspect the invention provides a method of detecting the presence of a DNA segment diagnostic for eucalyptus Tg event No:49 DNA in a sample, said method comprising:
a) contacting said sample with the DNA molecule of the invention;
b) subjecting said sample and said DNA molecule to stringent hybridization conditions; and
c) detecting hybridization of said DNA molecule to said DNA in said sample,
wherein said detection is diagnostic for the presence of said eucalyptus Tg event No:49 DNA in said sample.

In a further aspect the invention provides a method of detecting the presence of a DNA segment diagnostic for eucalyptus Tg event No:49 DNA in a sample, said method comprising:
a) contacting said sample with the pair of DNA molecules of the invention as primers;
b) performing an amplification reaction sufficient to produce a DNA amplicon; and
c) detecting the presence of said DNA amplicon in said reaction,
wherein said DNA amplicon comprises the nucleotide sequence selected from the group consisting of SEQ ID NO:48, SEQ ID NO:49, SEQ ID NO:50, SEQ ID NO:51, SEQ ID NO:52, SEQ ID NO:53 and SEQ ID NO:54.

In a further aspect the invention provides a eucalyptus plant, part thereof, tissue thereof or cell thereof comprising eucalyptus Tg event No:49 DNA characterized by the detectable presence of the recombinant DNA molecule of the invention.

In one embodiment the eucalyptus plant, part thereof, tissue thereof or cell thereof is insecticidal when provided in the diet of an insect pest.

In one embodiment the insect pest is selected from the group consisting of Thyrinteina arnobia (Geometridae), Physocleora dukinfeldia (Geometridae), Sarsina violascens (Erebidae), Glena spp. (Geometridae), Melanolophia consimilaria (Geometridae), Eacles spp. (Satumiidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

In a further embodiment the insect pest is selected from the group consisting of Thyrinteina arnobia (Geometridae), Thyrinteina leucocerae (Geometridae), Physocleora dukinfeldia (Geometridae), Sarsina violascens (Eribidae), Oxydia vesulia (Geometridae), Melanolophia consimilaria (Geometridae), and Spodoptera cosmioides (Noctuidae).

In a further embodiment the plant is further defined as progeny of any generation of a plant comprising the eucalyptus Tg event No:49.

In a further aspect the invention provides a method for protecting a eucalyptus plant from insect infestation, wherein said method comprises providing in the diet of an insect pest an insecticidally effective amount of cells or tissue of the plant of the invention.

In one embodiment said insect pest is selected from the group consisting of Thyrinteina arnobia (Geometridae), Physocleora dukinfeldia (Geometridae), Sarsina violascens (Erebidae), Glena spp. (Geometridae), Melanolophia consimilaria (Geometridae), Eacles spp. (Satumiidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

In a further embodiment said insect pest is selected from the group consisting of Thyrinteina arnobia (Geometridae), Thyrinteina leucocerae (Geometridae), Physocleora dukinfeldia (Geometridae), Sarsina violascens (Eribidae), Oxydia vesulia (Geometridae), Melanolophia consimilaria (Geometridae), and Spodoptera cosmioides (Noctuidae).

In a further aspect the invention provides a method of producing an insect resistant eucalyptus plant comprising:
a) breeding two different eucalyptus plants with at least one of the two different eucalyptus plants comprising the recombinant DNA molecule of the invention to produce progeny;
b) confirming in said progeny the presence of the recombinant DNA molecule; and
c) selecting said progeny comprising the recombinant DNA molecule;
wherein said progeny of step c) are insect resistant.

In a further aspect the invention provides a eucalyptus plant part or tissue comprising a detectable amount of a recombinant DNA molecule of the invention.

In a further aspect the invention provides a nonliving eucalyptus plant material comprising a detectable amount of a DNA molecule of the invention.

### CROSS REFERENCE TO RELATED APPLICATION

The contents of U.S. provisional patent application No. 63/310,518, filed 15 Feb. 2022, is incorporated herein by reference in its entirety.

### BRIEF DESCRIPTION OF DRAWINGS

FIG. 1: A schematic depiction of a plant-compatible construct used for the expression of single-Bt PP (represented as encoded by Gene A).

FIG. 2: Percent mortality of Thyrinteina arnobia (T. arnobia) caterpillars after 5 days of feeding on green-tissue derived from Eucalyptus transgenic (Tg) plants of several different transgenic events (Tg event No.s), expressing Cry2Aa

FIG. 3: Percent mortality of T. arnobia caterpillars after 5 days of feeding on green-tissue derived from further Eucalyptus transgenic events (Tg event No.s) expressing Cry1Ab.

FIG. 4: Percent mortality of T. arnobia caterpillars after 5 days of feeding on green-tissue derived from further Eucalyptus transgenic events (Tg event No.s) expressing Cry1Bb.

FIG. 5: Percent mortality of T. arnobia caterpillars after 5 days of feeding on green-tissue derived from background control non-transgenic Eucalyptus plants.

FIG. 6: Depiction of herbivorous activity on Eucalyptus leaves: Illustrates percent (%) surface area loss as a quantification of damage to leaves by caterpillars, and as a proxy of plant resistance to herbivorous activity.

FIG. 7: Depiction of herbivorous activity to Eucalyptus leaves: exemplary leaves showing varying damage percentages.

FIG. 8: Exemplary leaf or seedling damage to Wild Type (W.t) plants compared to Triple-Bt-Tg (Tg) plants (Tg event No. 49). Leaf damage (FIG. 8a, upper panel) or seedling damage (FIG. 8b, lower panel) inoculated with 3 or 10 2^{nd} instar T. arnobia caterpillar larvae on leaves and seedlings, respectively, observed at time 0 and after 6 days.

FIG. 9: A schematic representation of a binary expression vector used to express a Triple Bt PP combination, including a pBI121-backbone, encoding Cry2Aa and Cry1Ab driven by a 35S promoter and Cry1Bb driven by a Eucalyptus Rubisco promoter variant.

FIG. 10: The scheme of the expression cassette between T-DNA borders of the expression vector provided in FIG. 9 above.

FIG. 11: A Western Blot (WB) of Cry2Aa, Cry1Ab and Cry1Bb Bt PP in protein samples taken from cultured shoot tissue of Triple-Bt-Tg (Tg event no. 38) and control (AEC0224). Expected-size bands are marked with arrowheads. Ponceau S staining is provided below as loading control.

FIG. 12: Alignment of 1) the activated protein (native) Cry2Aa AA sequence (SEQ ID NO: 1), 2) the native Bt nucleic acid sequence (SEQ ID NO: 12) encoding the activated Cry2Aa protein, and 3) the nucleic acid sequence (of 2) optimized for expression in Eucalyptus (SEQ ID NO: 2).

FIG. 13: Alignment of 1) the activated protein Cry1Ab (truncated) AA sequence (SEQ ID NO: 3), 2), the native Bt nucleic acid sequence (SEQ ID NO: 42) encoding the activated Cry1Ab protein and 3) the nucleic acid sequence (of 2) optimized for expression in Eucalyptus (SEQ ID NO: 4).

FIG. 14: Alignment of 1) the activated protein Cry1Bb (truncated) AA sequence (SEQ ID NO: 5), 2) the native Bt nucleic acid sequence (SEQ ID NO: 44) encoding the activated Cry1Bb protein, and 3) the nucleic acid sequence (of 2) optimized for expression in Eucalyptus (SEQ ID NO: 6).

FIG. 15: List of transgenic events (Event name), CRY proteins (Bt PP line Expressed) and their respective Eucalyptus background clones (Background Clone).

FIG. 16: Caterpillar confinement. Cages installed in the field

FIG. 17: A—Removal of the branch for evaluation; B—Count of live caterpillars; C—Visual comparison between treatments.

FIG. 18: Average mortality of 1st instar caterpillars in treatments Tg event no. 49 (TR01), FGN-K (TR02) and FGN-K+Dipel (TR03) in Angatuba/SP. Values refer to the means of the 5 repetitions after 7 days of experimentation. Different letters represent statistically significant differences between treatment means by Tukey's 5% test. FGN-K is wildtype clone AEC0224.

FIG. 19. Average mortality of 1st instar caterpillars in treatments Tg event no. 49 (TR01), FGN-K (TR02) and FGN-K+Dipel (TR03) in Ibaté/SP. Values refer to the means of the 5 repetitions after 7 days of experimentation. Different letters represent statistically significant differences between treatment means by Tukey's 5% test.

FIG. 20. Average mortality of 1st instar caterpillars in treatments Tg event no. 49 (TR01), FGN-K (TR02) and FGN-K+Dipel (TR03) in Tres Lagoas/MS. Values refer to the means of the 5 repetitions after 7 days of experimentation. Different letters represent statistically significant differences between treatment means by Tukey's 5% test.

FIG. 21: Methodology with the use of microtubes, a caterpillar by microtube.

FIG. 22: Mortality results of T. arnobia when exposed to different interactions of Cry proteins

FIG. 23: Mean lethality rate in T. arnobia under different levels of doses of cry protein-prepared diets. Lowercase letters compare proteins within each dose and uppercase letters compare doses within each protein.

FIG. 24: Competition binding with 125I-Cry1Ab to T. arnobia BBMV. 0.1 nM of 125I-Cry1Ab in the presence of increasing excess (5, 10, 30, 50, 100, 300, 500 and 1000-fold) of unlabeled Cry1Ab, Cry2Aa and Cry1Bb as competitors.

FIG. 25: Competition binding with biotin-labeled Cry1Bb to T. arnobia BBMV. 22 nM biotin-Cry1Bb in the presence of 300 fold of unlabeled Cry1 Ab, Cry2Aa and Cry1Bb (left panel) or increasing excess (10, 50, 100, 300, and 500-fold) of unlabeled Cry1Ab, Cry2Aa as competitors (right panel). Total binding (TB) show the biotin-labeled Cry1Bb binding with no competitor.

FIG. 26: Competition binding with biotin-labeled Cry2Aa to T. arnobia BBMV. 22 nM biotin-Cry2Aa in the presence of increasing excess (10, 50, 100, 300, 500 and 1000-fold) of unlabeled Cry1Bb, Cry1Ab as competitors. Total binding (TB) show the biotin-labeled Cry2Aa binding with no competitor.

FIG. 27: A schematic representation of a model for the binding of Cry1Ab, Cry1Bb, and Cry2Aa to the midgut membrane of T. arnobia.

FIG. 28: Tg event no. 49 insert and genomic flanking region map.

FIG. 29: Insert elements with the nucleotide sequence of Tg event no. 49

FIG. 30: Sequence alignment of the insertion site in Tg event no. 49 (Tg event no. 49 site), clone AEC0224. Insert allele: The allele in which the t-DNA was inserted. Second allele: The second allele of this genetic locus. Grey shading indicates sequence identity. Insert site is indicated in white box. Analysis was done by MacVector software (wvw.macvector.com).

FIG. 31: Tg event no. 49 insert location based on the reference genome database.

FIG. 32: is a graphical depiction of the orientation and alignment of the DNA elements/segments that are present within the nucleotide sequence shown in SEQ ID NO: 48, which is the sequence of the inserted transgenic DNA and the corresponding adjacent 5′ and 3′ sequences of the Eucalyptus genome present within the Tg event No. 49.

The figure illustrates the physical arrangement of the junction sequences, arranged from 5′ to 3′, relative to SEQ ID NO: 48. The junction sequences of Tg event 49 may be present as part of the genome of a plant, seed, or cell containing Tg event 49. The identification of any one or more of the junction sequences in a sample containing DNA from a Eucalyptus plant, plant part, seed, or cell indicates that the DNA was obtained from Eucalyptus containing Tg event 49.

The presence of junction sequences for Tg event 49 may be demonstrated by a sequence from the group consisting of SEQ ID NO:49, SEQ ID NO:50, SEQ ID NO:51, SEQ ID NO:52, SEQ ID NO:53 and SEQ ID NO:54. The junction sequences may be indicated by the nucleotide sequences provided as SEQ ID NO:49 (5′ junction sequence) and SEQ ID NO:50 (3′junction sequence). Alternatively, the junction sequences may be indicated by the nucleotide sequences provided as SEQ ID NO:51 (5′ junction sequence) and SEQ ID NO:52 (3′ junction sequence) or the junction sequences may be indicated by the nucleotide sequences provided as SEQ ID NO: 53 (5′junction sequence) and SEQ ID NO:54 (3′junction sequence). In addition, any polynucleotide comprising a sequence complementary to any of the sequences described within SEQ ID NO 48 is within the scope of the invention.

FIG. 33: PCR amplification of an amplicon diagnostic for Tg event No. 49, as described in Example 17.

### DETAILED DESCRIPTION OF THE INVENTION

Bacillus thuringiensis pesticidal proteins (Bt PP) are known for their ability to deter and kill plant-pests, and transgenic expression of Bt PP has been used to render plants less susceptible to pest damages. Different insect pests may be either sensitive or resistant to the pesticidal effect of a specific Bt PP. There are hundreds of different Bt PP—and, accordingly, there remains a standing need to identify which Bt PP and which Bt PP combinations are potent against specific groups of insects, and have ample insecticidal activity, e.g., against insects in the order Lepidoptera.

Bacillus thuringiensis (Bt), (e.g. Bacillus thuringiensis Berliner, ATCC 10792), is a gram-positive, ubiquitous, spore-forming soil bacterium. During the sporulation stage of Bt, the bacteria produce crystal (Cry) proteins, which have selective insecticidal activity. The insecticide activity and its usefulness were indicated upon identification of Bt strain by Krieg et al., 1983 and Krieg et al. 1984 (described in U.S. Pat. No. 4,766,203, incorporated herein by reference), disclosing the toxicity against larvae of the coleopteran insects Agelastica alni (blue alder leaf beetle) and Leptinotarsa decemlineata (Colorado potato beetle). U.S. Pat. No. 4,797,279 disclosed a Bt tenebrionis and Bt kurstaki conjugated by means of microbiology to produce a hybrid strain and having in parallel both bi-pyramidal (kurstaki-) and cubic (tenebrionis-) crystals, and showing functionality in lepidopteran larvae mortality. It is commonly considered that the efficiency of a specific Bt PP is related to the interaction between the Bt PP and the proteins in the epithelial cells in a given insect's mid-gut—such as that genetic differences dictate the tropism and spectrum of a Bt PP (I.e. the insecticidal activity range of target insects of an individual Bt PP). In order to evaluate the potency of select Bt PP to protect against insect species, the applicants performed many of the experiments presented using T. arnobia at the larval stage.

The insect species (of the Lepidoptera order) Thyrinteina arnobia (arnobia t.: MONA No. 6772) is a geometrid moth found in North, Central and South America. The species includes (but may not be limited to) the subspecies: Thyrinteina arnobia arnobia; Thyrinteina arnobia phala Rindge; Thyrinteina arnobia picta Rindge; Thyrinteina arnobia quadricostaria Herrich-Schaffer; Thyrinteina arnobia tephra Rindge.

A composition or a method may be considered useful for protecting plants from damage by pests, such as insect pests, if for example, less damage is generated by the pest to a plant or plants (in the lab, greenhouse or in the field) following deposition of the composition on- or in the proximity of the plant(s); or after practicing the method in relation to the plant(s).

A composition or a method may be considered useful for inhibiting, killing or managing insect pests if, for example, in either laboratory, greenhouse, or field conditions—reduced population-size or population-growth of the pest is observable, more cases of dead insects are observable, less insects of advanced-life-stages are observable, smaller body-size of exposed insects are observable, and the like.

A common mechanism of resistance to Bt PP that has been observed in insect pests is linked to reduced toxin binding to brush border membrane vesicles (BBMV), due to mutations in receptor protein genes or mutations that result in reduced expression of receptor protein genes, thus affecting binding of Bt PP. Thus, an insecticidal Bt PP-agent (such as a transgenic plant expressing Bt PP) comprising more than one Bt PP is advantageous, as it is expected to greatly reduce the probability of evolution-based resistance of insects to multiple insecticidal Bt PP simultaneously (See also—U.S. Pat. No. 6,033,874, incorporated herein by reference in its entirety), and as described below. As described herein, the method utilizes a Triple Bt PP combination.

Without being bound by theory, a suggested mechanism/mode of action (MOA) for Bt PP insecticidal activity, following ingestion by an insect, comprises binding of a Bt PP to unique and specific midgut receptors followed by formation of cation-selective channels in the midgut cell membranes. In this manner, Bt PP disrupt the normal function of the midgut, eventually leading to the death of the insect.

Insect avoidance of Bt PP toxicity can evolve following genetic changes that lead to a reduction in Bt PP-binding and activity. Examples for such genetic changes are mutations, allelic variations, and/or knockout or knockdown of a specific receptor, any of which can lead to reduced toxicity of any potential PPs that bind to that receptor as a part of their MOA. Thus, it would be advantageous if a plant expressed more than one Bt PP, each of which recognizes their own unique receptor such that a mutation in a given receptor for a first PP, for example, would not eliminate the plant's resistance to the insect since the remaining Bt PPs would continue to be toxic to the targeted pest, making moot the evolving resistant population to the first PP.

In order to address the question of whether two or more Bt PPs of interest share receptors and, if so, their affinity to specific midgut receptors or, alternatively, have explicitly discrete Modes of Action (MOA), competition assays using labeled proteins can be performed to elucidate these characteristics.

A common and sensitive method to test for sharing of midgut receptors between two distinct Bt PP is the performance of competition assays using labeled proteins; see U.S. Pat. No. 9,567,602, incorporated herein by reference in its entirety.

Insects against which the present invention is directed may be from any species. In one embodiment the insects are from the order Lepidoptera.

In a further embodiment the insects are selected from at least one of the following families or tribes: Geometridae, Erebidae, Satumiidae, Arctiidae, Riodinidae and Notodontidae.

In one embodiment the Geometridae insects are selected from at least one of the following genera: Thyrinteina, Physocleora, Glena, Melanolophia, Oxydia and Iridopsis.

In one embodiment the insects from the Thyrinteina genus are selected from Thyrinteina arnobia and Thyrinteina leucocerae. Preferably the insect is from Thyrinteina arnobia. The Thyrinteina arnobia may in the following subspecies: Thyrinteina arnobia arnobia; Thyrinteina arnobia phala Rindge; Thyrinteina arnobia picta Rindge; Thyrinteina arnobia quadricostaria Herrich-Schaffer and Thyrinteina arnobia tephra Rindge.

In one embodiment the insects from the Physocleora genus is Physocleora dukinfeldia.

In one embodiment the insect from the Melanolophia genus is Melanolophia consimilaria.

In one embodiment the insect from the Oxydia genus is Oxydia vesulia.

In one embodiment the Erebidae insects are selected from the Sarsina genus.

In one embodiment the insect from the Sarsina genus is Sarsina violascens.

In one embodiment the Satumiidae insects are genus Eacles spp.

In one embodiment the Arctiidae insects are from the genus Eupseudosoma.

In one embodiment the insects from the Eupseudosoma genus are selected from Eupseudosoma aberrans and Eupseudosoma involuta.

In one embodiment the Riodinidae insects are from the genus Euselasia.

In one embodiment the insects from the Euselasia genus is Euselasia apisaon.

In one embodiment the Notodontidae insects are from the genera Nystalea and Spodoptera In one embodiment the insects from the Nystalea genus is Nystalea nyseus.

In one embodiment the insects from the Spodoptera genus is Spodoptera cosmioides.

In the context of this application, “transgenic co-expression” of proteins refers to expression in the same plant; there is not a requirement to have co-expression of all three polypeptides in the same cell, unless specified otherwise.

Suitable plants that can be used in the transgenic technology described herein include, as non-limiting examples, woody plants (e.g., perennial plants having an elongated hard lignified stem; i.e., trees), such as Eucalyptus, poplar, pine, fir, spruce, acacia, sweet gum, ash, birch, oak, teak, mahogany, sugar and Monterey, nut trees, e.g., walnut and almond, and fruit trees, e.g., apple, plum, cherry, citrus and apricot.

Other notable non-limiting examples of plants in which the methods described herein can be practiced include alfalfa, artichoke, arugula, asparagus, avocado, banana, barley, beans, beet, blackberry, blueberry, broccoli, Brussel-Sprouts, cabbage, canola, cantaloupe, carrot, cassava, cauliflower, celery, cilantro, coffee, corn, cotton, cucumber, duckweed, eggplant, endive, escarole, fennel, gourd, Indian mustard, safflower, olive, rice, barley, sugarcane, wheat, sorghum, sweet sorghum, and duckweed. Eucalyptus and related plants are of specific interest.

Relevant tree subtypes amendable for use in the methods described herein include but are not limited to Eucalyptus and pine species, including, for example, Eucalyptus (such as Eucalyptus grandis) and its hybrids, and Pinus subtypes. For example: Eucalyptus alba, Eucalyptus bancrofiti, Eucalyptus botryoides, Eucalyptus bridgesiana, Eucalyptus calophylla, Eucalyptus camaldulensis, Eucalyptus citriodora, Eucalyptus cladocalyx, Eucalyptus coccifera, Eucalyptus curtisii, Eucalyptus dairympleana, Eucalyptus deglupta, Eucalyptus delagatensis, Eucalyptus diversicolor, Eucalyptus dunnii, Eucalyptus ficifolia, Eucalyptus globulus, Eucalyptus gomphocephala, Eucalyptus gunnii, Eucalyptus henryi, Eucalyptus laevopinea, Eucalyptus macarthurii, Eucalyptus macrorhyncha, Eucalyptus maculata, Eucalyptus marginata, Eucalyptus megacarpa, Eucalyptus melliodora, Eucalyptus nicholii, Eucalyptus nitens, Eucalyptus nova-angelica, Eucalyptus obliqua, Eucalyptus occidentalis Eucalyptus obtusiflora, Eucalyptus oreades, Eucalyptus pauciflora, Eucalyptus polybractea, Eucalyptus regnans, Eucalyptus resinfera, Eucalyptus robusta, Eucalyptus rudis, Eucalyptus saligna, Eucalyptus sideroxylon, Eucalyptus stuartiana, Eucalyptus tereticornis, Eucalyptus torelliana, Eucalyptus urnigera, Eucalyptus urophylla, Eucalyptus viminalis, Eucalyptus viridis, Eucalyptus wandoo, and Eucalyptus youmanni, OR Pinus banksiana, Pinus brutia, Pinus caribaea, Pinus clasusa, Pinus contorta, Pinus coulteri, Pinus echinata, Pinus eldarica, Pinus ellioti, Pinus jeffreyi, Pinus lambertiana, Pinus massoniana, Pinus monticola, Pinus nigra, Pinus palustrus, Pinus pinaster, Pinus ponderosa, Pinus radiata, Pinus resinosa, Pinus rigida, Pinus serotina, Pinus strobus, Pinus sylvestris, Pinus taeda, Pinus virginiana, or Abies amabilis, Abies balsamea, Abies concolor, Abies grandis, Abies lasiocarpa, Abies magnifica, Abies procera, Chamaecyparis lawsoniona, Chamaecyparis nootkatensis, Chamaecyparis thyoides, Juniperus virginiana, Larix decidua, Larix laricina, Larix leptolepis, Larix occidentalis, Larix siberica, Libocedrus decurrens, Picea abies, Picea engelmanni, Picea glauca, Picea mariana, Picea pungens, Picea rubens, Picea sitchensis, Pseudotsuga menziesii, Sequoia gigantea, Sequoia sempervirens, Taxodium distichum, Tsuga canadensis, Tsuga heterophylla, Tsuga mertensiana, Thuja occidentalis, Thuja plicata.

Insecticides based on cry-gene Bt PP are known and available commercially. For example, DiPel® Df biological insecticide (EPA Reg. No 73049-39) includes the active ingredient of Bt subtype Kurstaki stein ABTS-351, and, specifically, fermentation solids, spores and Bt PP, all derived from a naturally occurring Bacillus strain that expresses full-length Bt PP (i.e., in contrast to only the activated portions). DiPel® Df is prepared into a spray composition by adding water followed by mechanical or hydraulic agitation. When using DiPel® as control in the assays described herein, 1 mL of DiPel® stock (obtained from “Sumitomo Chemical Co., Ltd, Japan”) was diluted into 1000 mL of distilled water. Bt PP have been historically named and categorized in several manners, as is known in the art; see Crickmore, N., Berry, C., Panneerselvam, S., Mishra, R., Connor, T. R. and Bonning, B. C. (2020). Bacterial Pesticidal Protein Resource Center, bpprc.org on the World Wide Web.

The methods described herein will employ, unless otherwise indicated, the conventional techniques in practicing of chemistry, biochemistry, microbiology, tissue culture, molecular biology, recombinant DNA technology and botany, which are within the skill of the art. Such techniques are known and explained fully in the literature.

Examples of literature which provides such techniques include: Langenheim and Thimann, (1982) Botany: Plant Biology and Its Relation to Human Affairs, John Wiley; Cell Culture and Somatic Cell Genetics of Plants, vol. 1, Vasil, ed. (1984); Stanier, et al., (1986) The Microbial World, 5^{th} ed., Prentice-Hall; Dhringra and Sinclair, (1985) Basic Plant Pathology Methods, CRC Press: Maniatis, et al., (1982) Molecular Cloning: A Laboratory Manual; DNA Cloning, vols. I and II, Glover, ed. (1985); Oligonucleotide Synthesis, Gait, ed. (1984); Nucleic Acid Hybridization, Hames and Higgins, eds. (1984); and the series Methods in Enzymology, Colowick and Kaplan, eds, Academic Press, Inc., San Diego, Calif.

As used herein, the term “DNA” or “DNA molecule” can refer to a double-stranded DNA molecule of genomic or synthetic origin. i.e., a polymer of deoxyribonucleotide bases or a polynucleotide molecule, read in the 5′ (upstream) to the 3′ (downstream) direction. As used herein, the term “DNA sequence” refers to the nucleotide sequence of a DNA molecule. The nomenclature used herein is that required by Title 37 of the United States Code of Federal Regulations § 1.822 and set forth in the tables in WIPO Standard ST.25 (1998), Appendix 2, Tables 1 and 3.

DNA elements useful for the expression of Bt PPs, as described herein include, for example, transgenic promoters derived by tools of molecular biology, and can be obtained by polymerase chain (PCR) amplification from a wide set of sources such as genomic sequences, plasmids and libraries of nucleic acid sequences, many of which are publicly available. Alternatively, such DNA elements can be synthesized chemically based on sequences described electronically in depositories. Such DNAs can be synthesized either in full or in portions and subsequently conjugated to generate the complete promoter.

The term “recombinant DNA” or “recombinant nucleotide sequence” is understood in the art to mean DNA that contains a genetically engineered modification through manipulation via mutagenesis, restriction enzymes, ligation, and the like.

The phrase “functional in a plant” such as for a nucleic acid that functions as a promoter can be taken to refer to the ability of that nucleic acid to drive expression in a plant nucleus when operably linked to a sequence to be expressed (e.g., a coding sequence). A promoter and expressed sequence are operably linked—when joined as part of the same nucleic acid molecule and suitably positioned and oriented for transcription to be initiated.

Expressed sequences in the context of the methods described herein can be expressed with or without an omega 5′ UTR sequence (“Ω”, a nucleic acid sequence transcribed into omega leader of TMV RNA). The omega leader of tobacco mosaic virus (TMV) is known in the art to provide enhanced translation of foreign RNAs both in vivo and in vitro; without being bound by theory, the omega leader of TMV may act as a translational enhancer in various cell types and different cell-free translational systems due to the transcript adopting a stable compact structure that avoids degradation.

For some applications, a functional recombinant DNA can be introduced into a non-specific location in a plant genome, which can be achieved by random genomic integration. In certain cases, a recombinant DNA construct can be introduced using site-specific integration. Both may be relevant to the methods described herein. Site-specific recombination systems include Cre-Lox as disclosed in U.S. Pat. No. 4,959,317; FLP-FRT as disclosed in U.S. Pat. No. 5,527,695; and site directed integration using CRISPR genome editing methods as disclosed in China patent application publication CN 107,142,282.

Several genome editing methods are known in the art. For example, CRISPR Genome Editing is the method of using the CRISPR-Cas system (derived from the prokaryotic acquired immune system) to enable site directed changes as well as site-selected insertions of larger elements into genomes. The methods described herein can be practiced using CRISPR site-directed insertion to insert a Bt PP sequence (a Cry coding sequence) into a specific location in the genome; to avoid negative positional effects when inserting a Bt PP-encoding sequence into the genome; or to edit a Bt PP sequence inserted into the genome after it has been incorporated there, and in order to, for example, refine the DNA sequence to be optimally expressed.

For example, sight directed insertion approaches can be used to insert the Bt PP-encoding constructs and expression cassettes described herein into the insertion site identified in Examples 16 and 17. Methods described herein, and exemplified in Example 17, can also be used to confirm correct insertion. Such methods can be used to produce a plant corresponding to Tg event No. 49 and/or a plant comprising the insertion locus (insert and genomic DNA flanking sequences) characteristic of the Tg event No. 49.

Adaptation of the CRISPR system to eukaryotes can be found in U.S. application Ser. No. 15/981,807 (to UCB) and U.S. Pat. No. 8,697,359 (Broad Institute), and the use of CRISPR in plants is provided, e.g., in US Pub. No. US 2020/0299717A1 (Ceres Inc.).

Numerous methods for introducing foreign genes into plants are known and can be used to insert a functional polynucleotide into a plant host, including biological and physical plant transformation protocols. See, e.g., Miki et al., “Procedure for Introducing Foreign DNA into Plants,” in Methods in Plant Molecular Biology and Biotechnology, Glick and Thompson, eds., CRC Press, Inc., Boca Raton, pp. 67-88 (1993).

The methods chosen can vary with the host plant, and can include chemical transfection methods such as calcium phosphate, microorganism-mediated gene transfer such as Agrobacterium (Horsch et al., Science 227:1229-31 (1985)), electroporation, micro-injection, and biolistic bombardment.

Expression cassettes and vectors and in vitro culture methods for plant cell or tissue transformation and regeneration of plants are known and available. See. e.g., Gruber et al., “Vectors for Plant Transformation,” in Methods in Plant Molecular Biology and Biotechnology, supra, pp. 89-119. Isolated polynucleotides or polypeptides can be introduced into a plant or a plant cell by one or more techniques typically used for delivery of nucleic acids into cells. Such protocols may vary depending on the type of organism, cell, plant or plant cell, i.e., monocot or dicot, targeted for gene modification. Suitable methods of transforming plant cells include microinjection (U.S. Pat. No. 6,300,543), electroporation (Riggs, et al., (1986) Proc. Natl. Acad. Sci. USA 83:5602-5606, direct gene transfer (Paszkowski et al., (1984) EMBO J. 3:2717-2722), and ballistic particle acceleration (U.S. Pat. No. 4,945,050), Agrobacterium mediated transformation (U.S. Pat. No. 5,981,840); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crosswa et al., (1986) Mol. Gen. Genet. 202: 179-185).

It is common to describe as ‘transgenic events’ different plants (or organisms) resulting from the same transformation; it is common that such events can differ in phenotype despite having the same genetic construct inserted into their genome: the reason commonly is considered to be each event having discrete genomic insertion points; thus, having different regulatory elements in their proximity which effect the extent of expression and the expression profile.

For this reason, differing phenotypes, particularly mildly different phenotypes, between transgenic events are not to be considered contradictory, and, specifically, the lack of a phenotype in a ‘transformation event’ may not be representative that the inserted element does not exert a function in regard to that phenotype; since the positional effect (in the genome) may render a transformed construct partially or effectively inactive.

In combination with the Bt PP described herein, a (i) selection marker or (ii) screenable markers may be used and expressed.
(i) Commonly used selective marker genes include those conferring resistance to antibiotics such as kanamycin (nptII), hygromycin B (aph IV) and gentamycin (aac3 and aacC4) or resistance/tolerance to herbicides such as glufosinate (bar or pat), glyphosate (epsps), and AMPA (phno). EPSPS (a cp4 epsps (aroA:CP4) as referred to herein) is a herbicide tolerant form of 5-enolpyruvulshikimate-3-phosphate synthase (EPSPS) enzyme that decreases binding affinity for glyphosate, thereby conferring increased tolerance to glyphosate herbicide. Examples of such selectable markers are illustrated in U.S. Pat. Nos. 5,550,318; 5,633,435; 5,780,708 and 6,118,047.
(ii) Screenable markers that provide an ability to visually identify transformants and expression such as the green fluorescent protein (GFP) and mCherry used as indicators of expression and expression level in the examples below and/or beta-glucuronidase or uidA gene (GUS) for which various chromogenic substrates are known. Fluorescence such as that acquired and measured by microscopic imaging is most commonly presented by (relative and) arbitrary units [A.u.].

The terms “isolated nucleic acid sequence” and “isolated DNA molecule” can be a nucleic acid or DNA molecule at least partially separated from other molecules normally associated with it in its native state (such as in a naturally occurring genomic sequence). In one embodiment, the term “isolated” is also used herein in reference to a nucleic acid or DNA molecule that is at least partially separated from nucleic acids that normally accompany the DNA molecule in its native state.

Thus, a nucleic acid or DNA molecule fused (or operably linked) to regulatory or coding sequences with which it is not normally associated, for example as the result of recombinant techniques, are considered herein to be isolated. Such molecules are considered isolated even when present, for example in the chromosome of a host cell, or within a plasmid construct in solution. The term “isolated” in this context encompasses molecules not present in their native state or context.

The term “primer” refers to a short polynucleotide, usually having a free 3′OH group, that is hybridized to a template and used for priming polymerization of a polynucleotide complementary to the target.

The term “probe” refers to a short polynucleotide that is used to detect a polynucleotide sequence that is complementary to the probe, in a hybridization-based assay.

Preferred probes for use in the present invention to identify recombinant DNA molecules corresponding to the insertion locus (insert and flanking genomic sequences) in Tg event No. 49, preferably span the junction between the insert and flanking genomic sequences at either the 5′ or 3′ end of the insert.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds. 1987, Molecular Cloning, A Laboratory Manual, 2nd Ed. Cold Spring Harbor Press; Ausubel et al., 1987, Current Protocols in Molecular Biology, Greene Publishing). Tm for polynucleotide molecules greater than about 100 bases can be calculated by the formula Tm=81, 5+0.41% (G+C−log (Na+). (Sambrook et al., Eds. 1987, Molecular Cloning, A Laboratory Manual, 2nd Ed. Cold Spring Harbor Press, Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length)° C.)

As used herein “sequence identity” relates to the extent to which two aligned polynucleotide or polypeptide sequences are identical throughout a window of alignment (e.g. window of alignment of nucleotides or amino acids). And wherein “optimally aligned” is in accordance with the criteria on which the algorithm is based.

An “identity fraction” for aligned segments of a test sequence and a reference sequence is the number of identical components that are shared by the two aligned sequences divided by the total number of components in reference sequence segment, i.e., the entire reference sequence or a smaller defined part of the reference sequence.

As used herein, the term “percent sequence identity” or “percent identity” refers to the percentage (%) of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than a given percent of the reference sequence over the window of comparison). Optimal alignment of sequences for aligning a comparison window are well known to those skilled in the art. A very common tool functional in this context is the BLAST algorithm, described below. In one embodiment percent identity is calculated over the full length of the recited sequence.

The Bt PP sequences described herein have been exemplified and disclosed based on their sequence. These sequences can be taken to be exemplary, as it would be understood that variant and equivalent Bt PP (and the nucleotide sequences encoding them), having a substantially similar sequence and thus similar functionality in the context of pesticidal and insecticidal activity, could be used.

Equivalent Bt PP can be taken to be those that share substantial amino acid homology. Substantial amino acid homology typically refers to greater than 90% (e.g., greater than 91%, 92%, 93%, 94% or 95%, or greater than 96%, 97%, 98%, or 99% identical in amino acid sequence).

Certain Bt PP have been exemplified and disclosed herein based on the nucleic acid sequences encoding the polypeptide sequence: it would be readily apparent to one of skill in the art that proteins, such as the Bt PPs disclosed herein, may be encoded by alternative sequences using different codons (e.g., codon degeneracy, codon optimization) that encode the same or substantially the same amino acids, as is known in the art.

The importance of codon optimization for transgenic expression when sequences are expressed in different organisms also has been recognized in the art, for example, using codons that have better efficiency (as they are more highly represented in the anti-codon tRNA pool) for a specific organism's genome, such as a plant (e.g., a Eucalyptus plant). Sequence optimization (optimization) can be used to avoid splice-sites and polyadenylation (poly-A) sites when, for example, a bacterial sequence is being expressed in a eukaryotic cell.

Those skilled in the art will understand that polynucleotides and constructs for expressing polypeptides in cells, plants and other organisms can include various other modifications including restriction sites, recombination/excision sites, codon optimisation, tags to facilitate protein purification, etc. Those skilled in the art will understand how to utilise such modifications, some of which may influence transgene expression, stability and translation. However, an art skilled worker would also understand that these modifications are not essential, and do not limit the scope of the invention unless particularly stated.

Variations of alternative sequences encoding the same protein sequences may be obtained by algorithms which ‘perform reverse transcription’ by relating amino acid sequences to the different codons which encode them. An example of an algorithm that identifies listed sequences using an encoded protein sequence of choice as a query—is ‘tblastn’ by NCBI (National Center for Biotechnology Information, available from: ncbi.nlm.nih.gov/on the World Wide Web) BLAST (basic local alignment search tool) is known in the art as an algorithm and program for comparing primary biological sequence information, such as the amino-acid sequences of proteins or the nucleotides of DNA and/or RNA sequences. BLAST is a registered trademark of the NCBI National Library of Medicine (National Center for Biotechnology Information. U.S. National Library of Medicine, 8600 Rockville Pike, Bethesda MD, 20894 USA).

For comparisons of protein sequences, the BLASTP is commonly used, and for the comparison of a DNA to a protein to which it may code, BLASTX (translated nucleotide sequences: BLASTX version 2.0) and BLASTN version 2.0 for reciprocally, identifying polynucleotide sequences which may translate to a protein query.

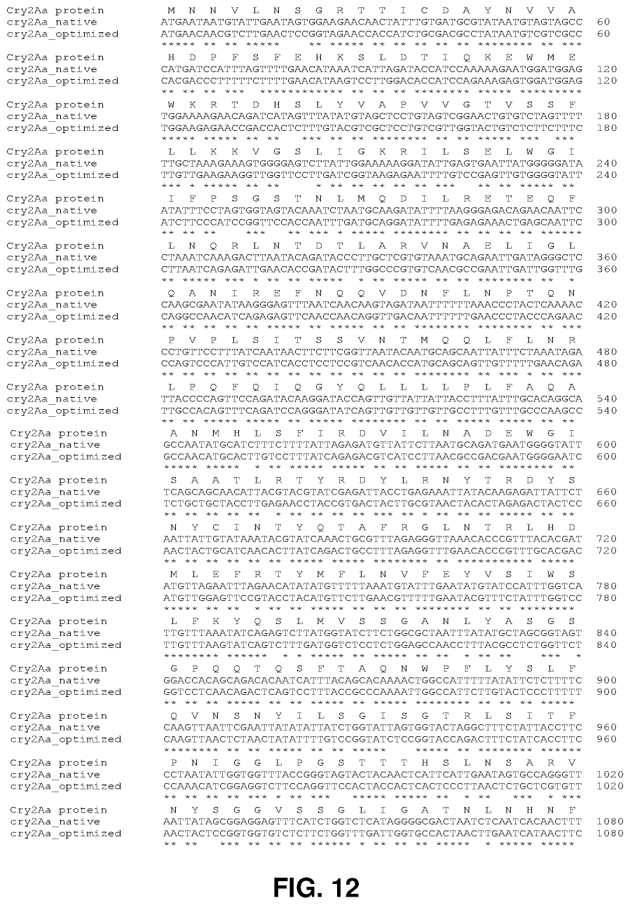

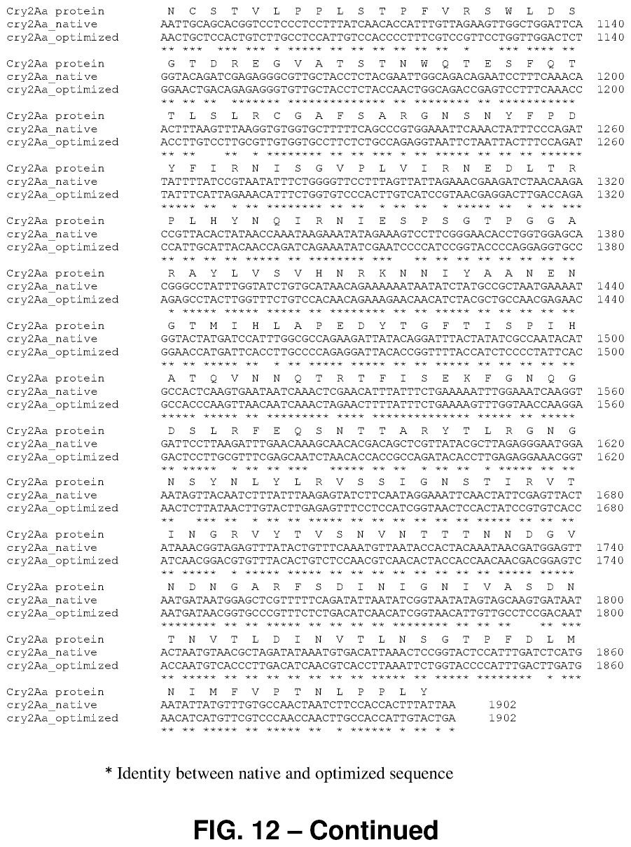

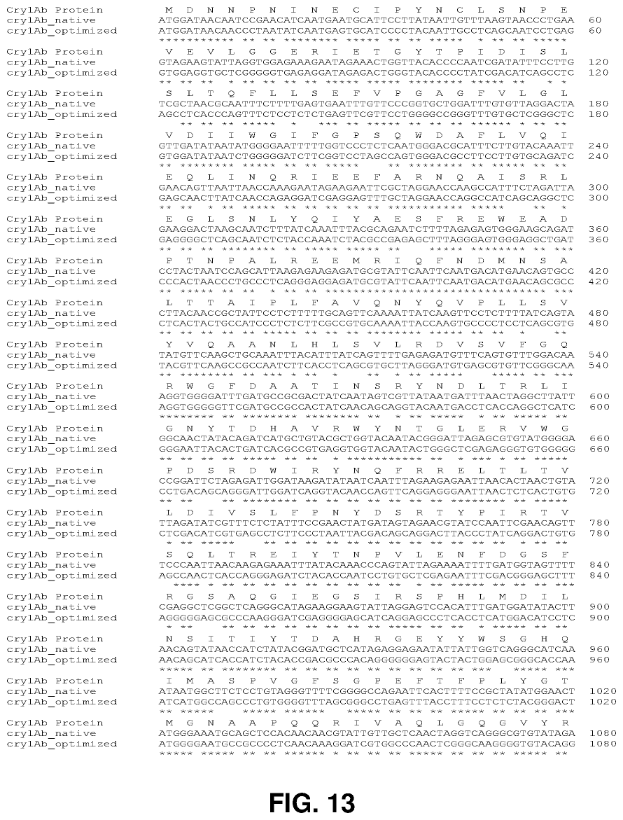

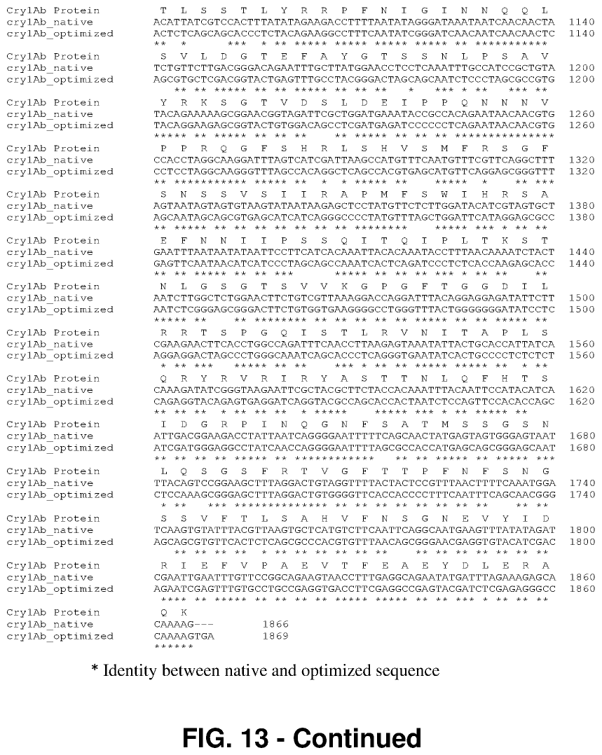

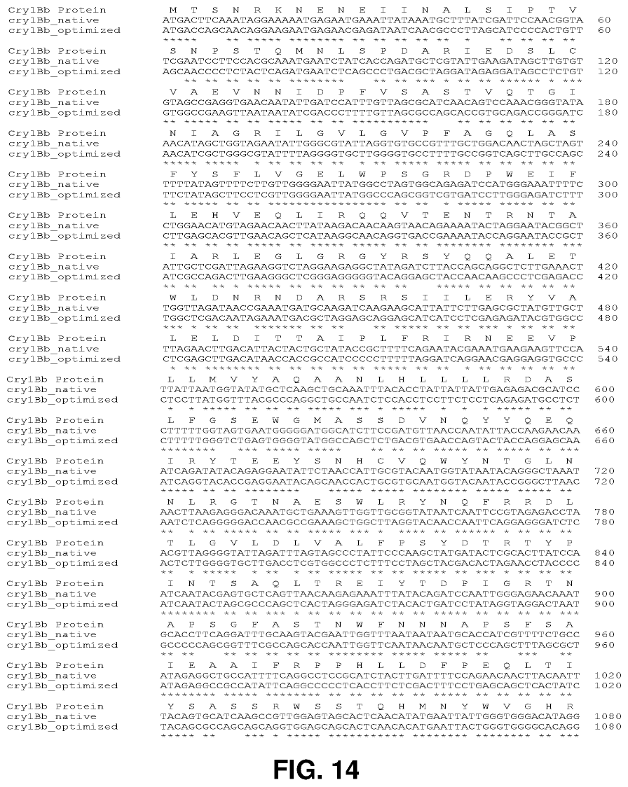

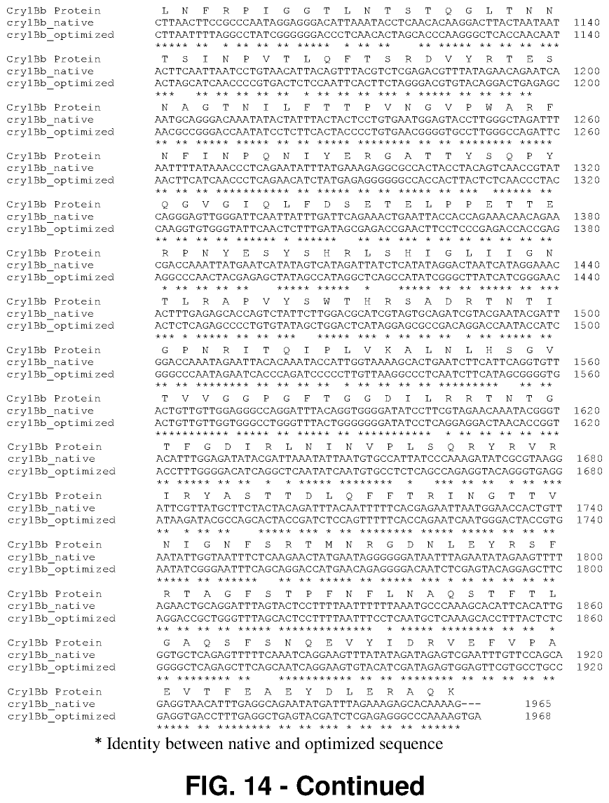

Proteins can be expressed transgenically from isolated nucleic acid sequences introduced into living cells by means of transgenic engineering or gene editing, as known in the art. Such expressed sequences need not be encoded by nucleic acid sequences from which they are found to be encoded originally. It is known in the art that different codons could provide for the expression of the same proteins sequence, and that such alternative codons may provide for better efficiencies in expression, if better compatible with the genome, tRNA pool or cell biology of the host cell, in which a non-endogenous sequence is to be expressed. Specifically, more efficient translation can be achieved by adapting the nucleic acid sequence to the codon usage (correlated to tRNA availability) of an organism, and even generally to a genus of the species (see, for example, U.S. Pat. No. 5,500,365, the content of which is incorporated herein by reference). For example, the insecticidal protein Bt PP of UniProt P0A377 (Cry2Aa; SEQ ID NO: 1), encoded in the Bt genome by SEQ ID NO:12, can be codon preference optimized, potential recombination sites removed and further optimized to reduce the number of polyadenylation signal sequences (ATTTA for example), while maintaining a gene that encodes the target protein, thus enabling more highly expressed target protein in the host plant (e.g., a dicot plant). Such a sequence can be encoded by, for example, SEQ ID NO:2 (codon preference optimized for Eucalyptus; FIG. 12). Similarly, the Cry1Ab Bt protein (UniProt entry P0A370; SEQ ID NO: 3), encoded by SEQ ID NO: 13, can be truncated for expression of an active Cry1Ab Bt PP insecticidal protein (see, e.g., SEQ ID NO: 42) or can be codon preference optimized and polyadenylation signal sequence reduced/optimized (see, for example, SEQ ID NO: 4, which has been codon optimized for Eucalyptus expression, FIG. 13); and the Cry1Bb protein (UniProt entry Q45739; SEQ ID NO: 5), encoded by SEQ ID NO: 14, can be truncated for expression of an active Cry1Bb Bt PP insecticidal protein (see, e.g., SEQ ID NO: 44) or can be codon preference optimized and polyadenylation signal sequence reduced/optimized (see, for example, SEQ ID NO: 6, which has been codon optimized for Eucalyptus expression; FIG. 14).

Nucleic acids as described herein may be identified by amplification using primers such as but not limited to those in Table 1 below:

TABLE 1

| | | | | | Amplicon |
|---|---|---|---|---|---|
| SEQ ID | | SEQ ID | | | expected |
| NO: | Forward Primer | NO: | Reverse Primer | Amplicon Sequence | size (bp) |
| 28 | ATGAACAACGTCTTG | 32 | TCAGTACAATGGTGG | cry2Aa CDS of | 1902 |
| | AACTCCGGTAG | | CAAGTTGGTTG | SEQ ID NO: 2 | |
| 29 | ATGGATAACAACCCT | 33 | TCACTTTTGGGCCCTC | cry1Ab CDS of | 1869 |
| | AATATCAATGAG | | TCGAGATC | SEQ ID NO: 4 | |
| 30 | ATGACCAGCAACAGG | 34 | TCACTTTTGGGCCCTC | cry1Bb CDS of | 1968 |
| | AAGAATGAG | | TCGAGATC | SEQ ID NO: 6 | |
| 31 | GCAAGAACCCCTTTTT | 35 | GAATTTCTAAAAATG | RbcS; | 7269 |
| | CAACCCTATG | | ACAAGTCAACAAATC | Eucgr.J01502.2 | |
| | | | | of SEQ ID NO: 8 | |

Using the primer sets above and genetic material derived from plants described herein (and specifically the Double or Triple-Bt Tg plants described herein) would provide amplicons having the sequences SEQ ID NO: 2, SEQ ID NO: 4 and SEQ ID NO: 6. (equivalent to the native Bacillus thuringiensis sequence SEQ ID NO: 12, a truncated version of the native sequence SEQ ID NO: 42 and a truncated version of the native sequence SEQ ID NO: 44, respectively. A person of skill in the art would understand that the plants co-expressing Bt PP as described herein also may co-express non-Bt PP proteins such as insecticidal proteins (or antifungal, antibacterial, or antiviral proteins) which confer additional resistance of the plant to pests; the simultaneous co-expression of multiple insecticidal proteins in plants is advantageous as it can minimize the possibility of developing pathogen resistant strains, and potentially result in a synergistic insecticidal effect.

In addition, co-expressing Bt PP as described herein along with herbicide resistant traits will enable the Bt PP plants as described herein to be exposed to herbicides used to kill competing plant without damages to the Bt PP plants described herein.

It would be understood that expressed Bt PP need not include the crystal (or crystallization-inducing) amino acid portion of the naturally occurring Cry proteins. The crystal portion has utility to the bacteria expressing the protein from an evolutionary perspective, but is not required to confer toxicity and pest resistance. The portion of the Bt Cry protein that confers toxicity and pest resistance is referred to as the “activated” portion. Thus, the term “Bt PP” should be understood to be a pesticidal protein which includes the “active” or “activated” portion of a Bacillus thuringiensis Cry protein, but may or may not include additional portions including the crystal portion.

The term “isolated protein” can be taken to be understood as a protein which is (at least partially) separated from other molecules normally associated with it in its native state; such as when synthesized outside of its naturally occurring environment; for example, when a protein is expressed in an orthologous transgenic system, synthesized chemically or in a cell-free system, or when a protein is purified from its naturally occurring environment.

Identification of expressed protein in plant tissue may be done by different methods known in the art, including Western blots (WB). Use of primary and secondary antibodies is known in the art.

Ponceau S is a negatively charged, red colored stain which binds to positively charged amino groups and non-polar regions of proteins. This stain has a sensitivity-detection limit of around 250 Nano-grams of protein following SDS-PAGE and electro-transfer to nitrocellulose membranes. Ponceau S can be used, for example, to confirm equal loading of proteins in different lanes in SDS-PAGE gels.

Crop protection is a major field in which the methods described herein may be used. One class of suitable crops are woody plants. In the context of this document, woody plants may be understood to be plants that produce wood as their structural tissue and, thus, have a firm stem. Wood is primarily composed of xylem cells with cell walls made of cellulose and lignin. Xylem is a vascular tissue which moves water and nutrients from the roots to the leaves. Most woody plants form new layers of woody tissue each year, and so increase their stem diameter from year to year, with new wood deposited on the inner side of a vascular cambium layer located immediately beneath the bark. Young woody plants are susceptible to insect pest, and when grown as a crop, suffer damages due to pest herbivorous activity. Managing and limiting pest damage is a significant challenge in these crops.

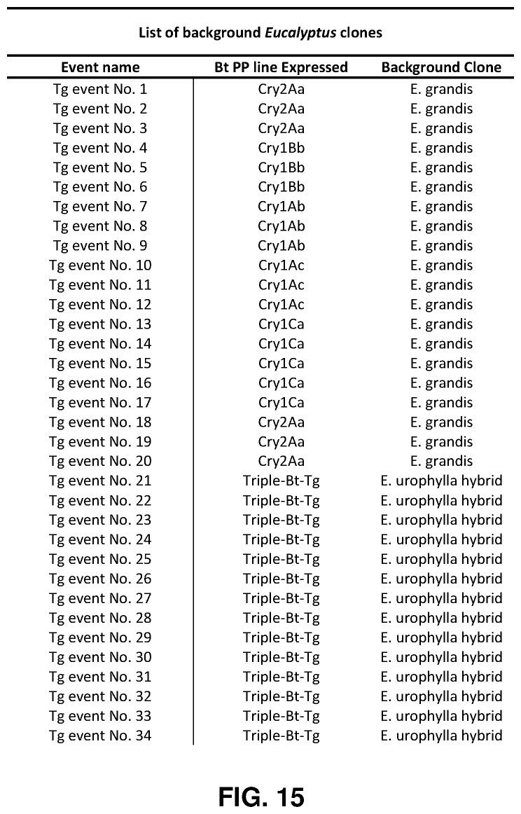

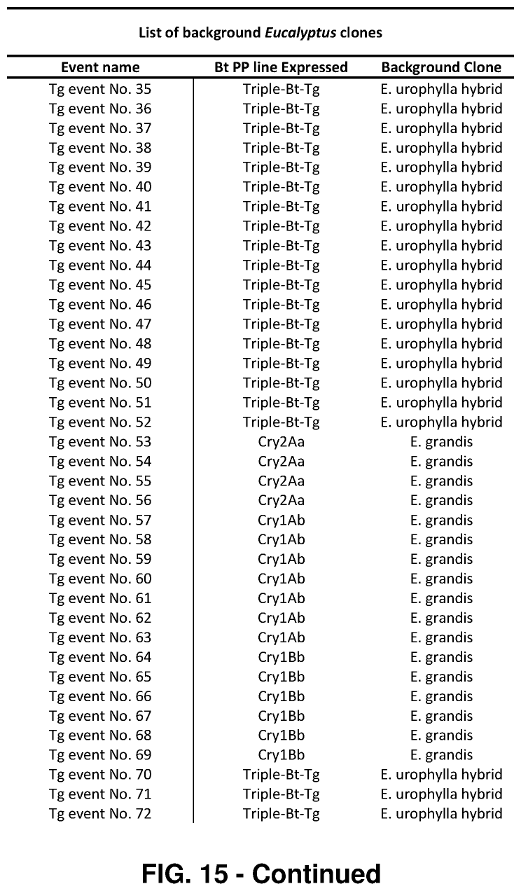

For example, defoliating insects are prominent harmful agents to trees, and one of the most prominent harmful agents to forests in Brazil. Of those agents, specifically Thyrinteina arnobia (T. arnobia), is a major pest of Eucalyptus in Brazil including the Eucalyptus background clones (FIG. 15) and Eucalyptus controls used in the experiments described herein. Defoliation affects tree growth by reducing the amount of photosynthetic tissue, which causes a direct reduction in the amount of carbohydrates available for growth and thus, reduced growth of the tree. Lepidoptera caterpillars devour the leaf blade.

For evaluation purposes, insects, including caterpillars, can be classified by size. Specifically, the size of caterpillars can be used as a proxy for toxicity and pest resistance: for example, caterpillars smaller than 1 cm in length can be designated as L1, caterpillars between 1-2 cm in length can be designated L2, caterpillars between 2-3 cm in length can be designated as L3, and caterpillars over 3 cm in length can be designated as L4. Such a classification system can be useful when comparing toxicity in a functional manner using diverse insect pests.

The artificial diet/growth medium (‘the diet’) used throughout this disclosure for assays with live caterpillars was prepared according to the recipe provided in Wilckenand and Berti Filho. 2006 (Brazilian journal of agriculture) cited below in Example 10. To make the recipe, ingredients are divided into groups and prepared in three basic steps. (1) Mixing the components of group a below in boiling water. (2) Adding in the components of group c, homogenizing followed by cooling the mixture to ˜25-40° C., and (3) adding the components of group b, wherein the three groups are: group a (wheat gene, brewer's yeast, cornflower, soybean meal, skimmed milk and soy oil) group b (Wesson salts, vitamin D, vanderzant, nipagin, ascorbic acid, sorbic acid) and group c (agar, water).

The invention will now be illustrated by the following non-limiting Examples.

### EXAMPLES

### Example 1: Cloning of Bt PP Coding Sequences into Plant-Compatible Expression Vectors

In order to clone single Bt PP into plant-compatible expression vectors—DNA coding sequences (CDS) encoding Bt PP were sequence optimized for Eucalyptus codon usage preferences, removal of potential recombination sites and polyadenylation sites and separately cloned into a pBI121 vector by conventional restriction-ligation methods.

Constructs were also cloned to include a NPTII CDS (similarly codon optimized for Eucalyptus expression). Each cloned Bt PP CDS was operably linked to a 35S-EucEF1-intron promoter, of SEQ ID NO: 27, constructed from a 35S CaMV constitutive promoter followed by the Eucalyptus EF1-intron (the Translation elongation factor EF-1 alpha/Tu) sequence).

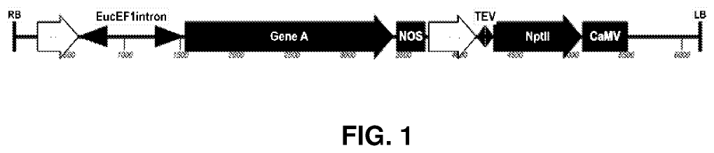

Bt PP which were cloned in this manner included: Cry1Ab (SEQ ID NO: 3, UniProt P0A370—corresponding to nucleic acid sequence SEQ ID NO: 4), Cry1Bb (SEQ ID NO: 5, UniProt Q45739—corresponding to nucleic acid sequence SEQ ID NO: 6), Cry2Aa (SEQ ID NO: 1, UniProt P0A377—corresponding to nucleic acid sequence SEQ ID NO: 2), Cry1Ac (SEQ ID NO: 37, corresponding to nucleic acid sequence SEQ ID NO: 38), Cry1Ca (SEQ ID NO: 39, corresponding to nucleic acid sequence SEQ ID NO: 40), and nucleic acid sequences cry1 Aa cry1Da, cry1Ea, cry1Fa, cry9Ca and cry9Ea. A schematic representation of a cloning vector for a single-Bt PP construct used for expression is provided in FIG. 1.

Intact cloning of Bt PP nucleic acid sequences into the expression vector was confirmed by PCR followed by electrophoresis, using the primer sequences provided in Table 2.

TABLE 2

| | Forward | Reverse | | Expected |
|---|---|---|---|---|
| | Primer | Primer | Target | size |
| Construct | SEQ ID NO: | SEQ ID NO: | Gene | (bp) |
| pET8Ndel-cry1Aa | 15 | 26 | cry1Aa | 699 |
| pET8Ndel-cry1Ab | 16 | 26 | cry1Ab | 623 |
| pET8Ndel-cry1Ac | 17 | 26 | cry1Ac | 708 |
| pET8Ndel-cry1Ca | 18 | 26 | cry1Ca | 599 |
| pET8Ndel-cry1Da | 19 | 26 | cry1Da | 719 |
| pET8Ndel-cry1Ea | 20 | 26 | cry1Ea | 683 |
| pET8Ndel-cry1Fa | 21 | 26 | cry1Fa | 721 |
| pET8Ndel-cry1Bb | 22 | 26 | cry1Bb | 556 |
| pET8Ndel-cry2Aa | 23 | 26 | cry2Aa | 552 |
| pET8Ndel-cry9Ca | 24 | 26 | cry9Ca | 580 |
| pET8Ndel-cry9Ea1 | 25 | 26 | cry9Ea | 1444 |

### Example 2: Transformation of Bt PP-Containing Constructs into Plants, Plant Regeneration, and Selection

Following cloning verification, the constructs were transformed into Eucalyptus background clone (see FIG. 15) tissue by A. tumefaciens strain LB A 4404, and transformed tissue was regenerated into Transgenic (Tg) plants.

The constructs were transformed as described in Prakash et al., 2009 and regenerated as provided in U.S. application Ser. No. 16/644,643, incorporated herein by reference in its entirety:

More specifically: shoots of Eucalyptus were propagated in-vitro on Murashige and Skoog medium (MS also called MSO or MSO (MS-zero)) basal salt medium consisting of 3% (w/v) sucrose and 0.8% (w/v) agar. All in-vitro plant materials were incubated at 25±2° C. for 16-h photoperiod with cool white fluorescent lamps with an intensity of 30 llEm-2 s-1. Agro bacterial culture collected at late log phase was pelleted and re-suspended in MS basal salt medium. Leaves from in-vitro material were collected and used as explants for transformation experiments. Explants were pre-cultured on the MS regeneration medium supplemented with 0.5 mg/l 6-Benzylaminopurine (BAP) and 0.1 mg/l NAA for 2 d. Later, pre-cultured Eucalyptus grandis leaf explants were gently shaken in the bacterial suspension for 10 min and blotted dry on a sterile filter paper. Explants were then cultivated in medium under the pre-culture conditions for two days. Following co-cultivation, explants were washed in MS liquid medium, blotted dry on a sterile filter paper, and transferred to MS regeneration medium containing 0.5 mg/l 6-Benzylaminopurine and 0.1 mg/l 1-Naphthaleneacetic acid supplemented with 40 mg/l, kanamycin and 300 mg/l cefotaxime. After 4-5 weeks of culture, regeneration was observed and explants were transferred to liquid elongation medium (MS medium supplemented with 0.5 mg/l BAP, 40 mg/l kanamycin, and 300 mg/l cefotaxime) on paper bridges. The elongated shoots (1.5-2 cm) were propagated on MS medium with 0.1 mg/l BAP, and leaf segments regenerated. Positive explants were grown on MS medium containing 0.04 mg/L BAP.

The procedure above was performed using each of the optimized sequences: cry1Aa, cry1Ab, cry1Ac, cry1Ca, cry1Da, cry1Ea, cry1Fa, cry1Bb, cry2Aa, cry9Ca and cry9Ga, generating transformation plant events that express each of the Bt PP separately.

### Example 3: Toxicity of Plant Tissue Derived from Transgenic Eucalyptus Plants Expressing Various Bt PPs to T. arnobia and Physocleora dukinfeldia

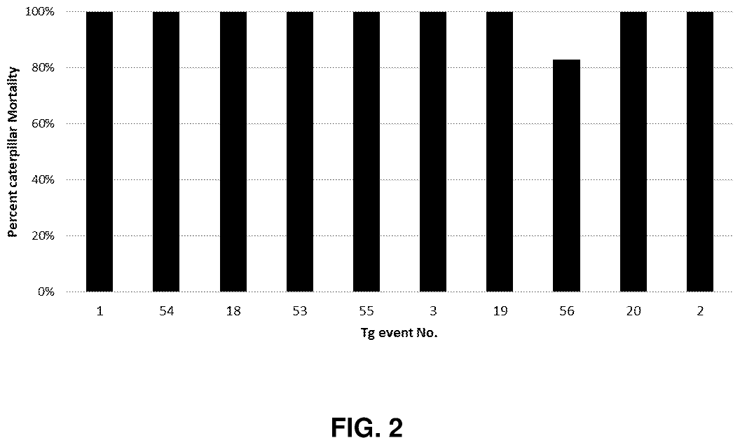

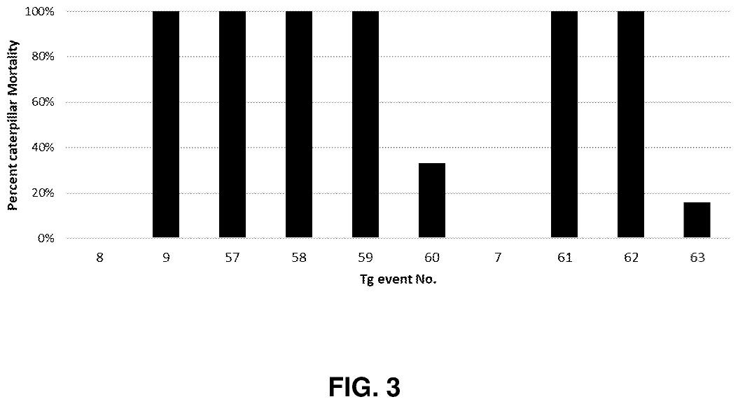

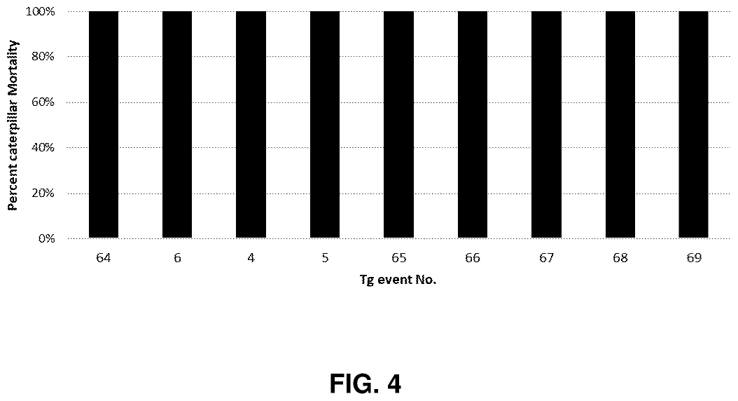

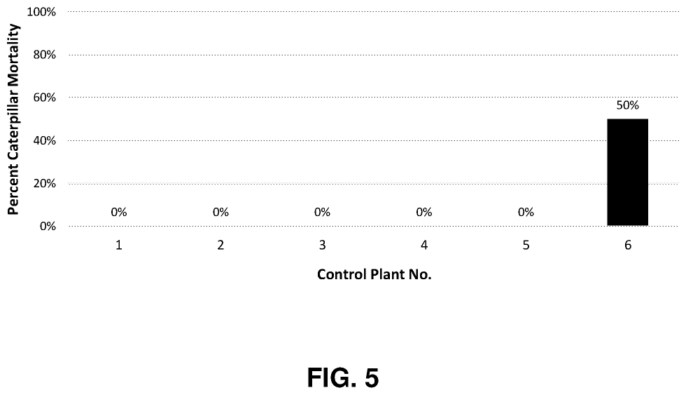

The evaluation of the toxicity of individual Bt PPs as plant-expressed proteins for inhibiting or killing insect pests was performed as follows: 1^{st} instar T. arnobia caterpillars were exposed to green-tissue derived from the Tg plants expressing a single Bt PP, described in Example 2 above. Specifically, slices of leaf tissue were placed on Whatman filter paper 1 mm, in plastic containers modified to facilitate gas exchange and avoid humidity formation: 3 caterpillars were introduced into each plastic container (including leaf material from a single Tg plant event, on Whatman filter paper 1 mm). Six non-transgenic, negative non-Tg control plants (clone ITA25, a background clone) were used. Percent caterpillar mortality after 5 days of exposure for: Cry2Aa, Cry1Ab and Cry1Bb and negative control plants, is provided in bar graphs in FIG. 2, FIG. 3, FIG. 4 and FIG. 5, respectively.

The evaluation of the toxicity of individual Bt PPs as plant-expressed proteins for inhibiting or killing insect pests was performed essentially as above: five 1st instar (first stage larvae) Physocleora dukinfeldia caterpillars were exposed to Tg plants expressing a single Bt PP, described above. Five larvae were introduced in each plastic container. Percent larvae mortality after 7 days of exposure to Tg Cry2Aa, Cry1Ab and Cry1Bb plants and negative control plants, is summarized in Table 3 below.

TABLE 3
Percent Mortality of Physocleora dukinfeldia
exposed to leaves expressing single Bt PPs

| | | Percent |
|---|---|---|
| | | mortality |
| Bt PP | Source Tg Event | after 7 days |
| Cry2Aa | Tg event No. 19 | 100% |
| | Tg event No. 55 | 100% |
| Cry1Bb | Tg event No. 5 | 100% |
| | Tg event No. 4 | 100% |
| Cry1Ab | Tg event No. 62 | 100% |
| Control | Non Tg control: (ITA 25) | 12% |

In additional experiments, the efficacy of the Bt PP in killing T. arnobia pests was assessed by assaying percent survival over time (Death Curve)—of T. arnobia caterpillars fed by plant green-tissue from either: Tg plants expressing one of several Bt PP (each represented by several independent transgenic events) or from control plants (ITA25) prepared as described above in Example 2.

Results are summarized in Table 4 below. Notably, for the Bt PP Cry2Aa, Cry1Bb and Cry1Ab (but not for Cry1 Ac or Cry1Ca), select transgenic events, for example Tg Events 2, 4, 5, 6, and 9, expressing one of the Bt PP tested, showed 0% survival of pest caterpillars within the various timeframes examined

TABLE 4
Percent survival over time (Death Curve)

| | Survival | 48 hs | 72 hs | 96 hs | 120 hs | 144 hs |
|---|---|---|---|---|---|---|
| Cry2Aa | Tg event No. 1 | 56% | 33% | 33% | 33% | 11% |
| | Tg event No. 2 | 33% | 11% | 0% | 0% | 0% |
| | Tg event No. 3 | 89% | 89% | 78% | 78% | 44% |
| Cry1Bb | Tg event No. 4 | 22% | 0% | 0% | 0% | 0% |
| | Tg event No. 5 | 11% | 11% | 0% | 0% | 0% |
| | Tg event No. 6 | 0% | 0% | 0% | 0% | 0% |
| Cry1Ab | Tg event No. 7 | 100% | 100% | 100% | 100% | 100% |
| | Tg event No. 8 | 100% | 100% | 100% | 100% | 100% |
| | Tg event No. 9 | 0% | 0% | 0% | 0% | 0% |
| Cry1Ac | Tg event No. 10 | 89% | 89% | 89% | 89% | 89% |
| | Tg event No. 11 | 100% | 100% | 100% | 100% | 100% |
| | Tg event No. 12 | 89% | 89% | 89% | 77% | 77% |
| Cry1Ca | Tg event No. 13 | 100% | 100% | 100% | 100% | 100% |
| | Tg event No. 14 | 100% | 100% | 100% | 100% | 100% |
| | Tg event No. 15 | 100% | 100% | 100% | 100% | 100% |
| | Tg event No. 16 | 100% | 100% | 100% | 100% | 100% |
| | Tg event No. 17 | 100% | 100% | 100% | 100% | 100% |
| Control | Non Tg control: | 97% | 97% | 97% | 97% | 97% |
| | (Ita 25) | | | | | |

### Example 4: Eucalyptus Transgenic Events Expressing Bt PP Confer Toxicity to, and Provide Protection Against, Damage by T. arnobia Caterpillars when Used as a Sole Source of Food

We next examined Bt PP toxicity to T. arnobia caterpillars when expressed in intact plants. For this experiment, we utilized modified container-cages that allow assaying individual young plants (plantlets). The modified cages included a lower portion of a container designed to hold the root portion of the plants immersed in water to keep the plantlets hydrated; and an upper portion made from a mesh-fabric screen—which allows air flow yet contains the insect caterpillars inside. 1^{st} instar T. arnobia caterpillars were maintained throughout the assay in these modified cages with a plantlet, limiting their diet to the plantlet plant tissue: either of a Cry2Aa Tg plants (described above), or from negative control plants (background clone ITA25), as a sole source of food, over a time period of 120 hours.

Table 5 below provides T. arnobia survival when provided with plantlets of 6 independent transgenic events expressing Bt PP Cry2Aa. 25 caterpillars were introduced into the cage of each plantlet.

TABLE 5
T. arnobia survival

| | 24 hs | 72 hs | 96 hs | 120 hs |
|---|---|---|---|---|
| Control plant | 100% | 100% | 100% | 100% |
| Tg event No. 1 | 100% | 24% | 20% | 20% |
| Tg event No. 18 | 80% | 0% | 0% | 0% |
| Tg event No. 53 | 88% | 32% | 24% | 24% |
| Tg event No. 19 | 88% | 0% | 0% | 0% |
| Tg event No. 20 | 96% | 0% | 0% | 0% |
| Tg event No. 2 | 92% | 12% | 12% | 12% |

Independent transgenic events (events 18, 19 and 20) resulted in death of 100% of caterpillars (0% survival) during the first 72 hrs, whereas other events (events 1, 53 and 2), while also being toxic, resulted in <25% survival after the full 120 hr interval. 100% of introduced caterpillars survived on the Controls plants.

### Example 5: Variability of Different Transgenic Events in Grown Plants Expressing Bt PP in Protecting Against T. arnobia

We examined the potency of the Cry2Aa-expressing transgenic events grown plants provided in the previous Examples against T. arnobia caterpillars.

Plants were maintained and grown in the greenhouse up to 8 months, thereafter they were placed in large modified container-cages with similar features to those described in Example 4 above, with obvious modifications for the size of the plants (concordantly, the experimental setup is referred to herein as “big pot” experiment, as the plants are kept in large pots). Specifically, caterpillars were counted for percent survival following a single interval of 7 days, in which they were given (as a sole source of food) the grown Bt PP-expressing Tg plants, or non-Tg control plants (ITA25).

Table 6 below provides:
(1) Survival: caterpillar-survival results for separate transgenic events expressing Cry2Aa in big pots;
(2) Excrement: quantification of pest excrement (feces, marked “Excrement (g)”), which is a proxy/indicator of herbivorous activity on the plant, quantitatively addressing resistance conferred by expressing Bt PP to transgenic plant material;
(3) Damage: damage to green tissue in transgenic events by percent (%) surface of leaf area eaten by caterpillars (marked “Damage”) and quantified as described below;
(4) Development: observed size differences and development-stage differences between the insects as indicated in the different treatments (marked “Development”).

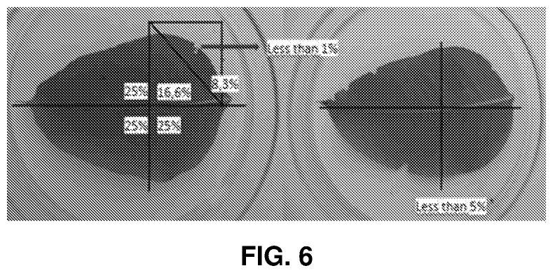

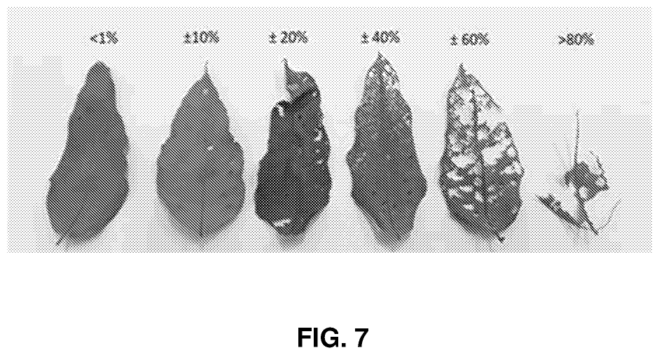

FIG. 6 shows how the calculation of leaf surface loss due to caterpillar feeding is performed and corresponds to the column labeled “Damage” in Table 6. FIG. 7 shows examples of different damage percentages. We defined percent surface damage (an indicator of resilience against the pest) into functional categories, in which leaves with <5% damage surface area were defined as resistant, leaves with 5-10% damage was defined as having medium resistance, and leaves with 11% or greater damage were defined as susceptible.

TABLE 6
T. arnobia greenhouse (“big pot”) results: Survival and damage

| | Survival | Excrement (gr) | Damage | Development |
|---|---|---|---|---|
| Tg event No. 1 | 50% | 2.5 | 40% | Big Size |
| Tg event No. 18 | 0% | 0 | 0% | N/a* |
| Tg event No. 53 | 30% | 1.3 | 30% | Medium size |
| Tg event No. 19 | 0% | 0 | 0% | N/a* |
| Tg event No. 20 | 0% | 0 | 0% | N/a* |
| Tg event No. 2 | 25% | 0.4 | 5% | Small size |
| Control plant | 33% | 6.2 | 100% | Starting pupae; |
| | | | | Big size |

*Not analyzed, no survivors

Caterpillar survival assays on (1) non-transgenic plant-derived tissue placed on Whatman filter paper 1 mm (2) plantlet and (3) mature plants (big pot experiments) all consistently showed high survival of caterpillars in control conditions, contrasted by low survival percentages of caterpillars exposed to specific Bt PP-expressing transgenic events: (e.g., to events 18, 19 and 20 in the Table above). The higher potency of Cry2Aa events numbered 18, 19 and 20, above, in contrast to other Tg events (e.g., Tg event No. 1), may be due to a positional effect of each transgenic event, i.e., expression level of the transgene and the resulting amount of expressed protein in a particular event may vary, depending on the location of the insertion within the genome. It is known in the art that some portions of a genome are more favorable in supporting higher expression levels.

### Example 6: Generating Transgenic Eucalyptus Plants Expressing a Triple Bt PP Combination

In order to generate plants resilient or resistant to insect pests, multiple Bt PP were cloned and co-expressed in Eucalyptus. Specifically, the following triple combination of Bt PP was expressed in Eucalyptus:
(I) Bt-Cry2Aa (UniProt P0A377; SEQ ID NO: 1)
(II) Bt-Cry1Ab (UniProt P0A370; SEQ ID NO: 3)
(III) Bt-Cry1Bb (UniProt Q45739; SEQ ID NO: 5).

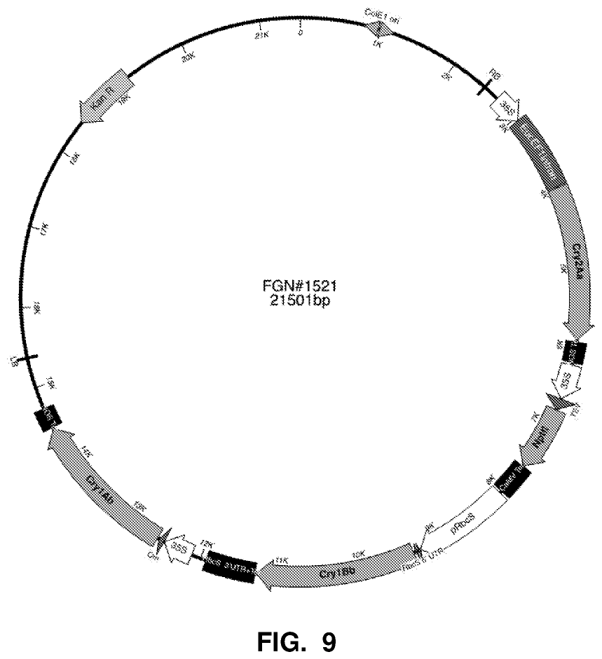

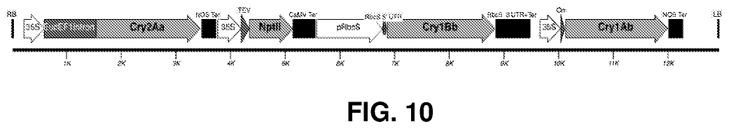

The construct harboring the three Bt PP is depicted in FIG. 9, and the DNA enclosed between T-DNA borders is provided graphically in FIG. 10.

To construct the expression vector of FIG. 9: the binary vector pBI121 (Clontech, Palo Alto CA; Chen et al., 2003) was modified and inserted with functional sequences as follows:
(i) A cassette expressing the Neomycin phosphotransferase II (nptII) gene, controlled by the NOS promoter and NOS terminator, was replaced by a synthetic DNA fragment containing a Eucalyptus optimized nptII coding sequence (CDS) under the control of the Cauliflower mosaic virus 35S promoter (CaMV 35S) fused to the Tobacco etch virus (TEV) translational enhancer and a downstream CaMV terminator. This cassette was cloned using the BstZI7I/PmeI-HindII restriction sites.
(ii) The beta-glucuronidase (GUS) expression cassette was excised out using the EcoRI sites.
(iii) A synthetic DNA expression cassette of Bt-cry2Aa gene controlled by CaMV 35S promoter fused to the Eucalyptus Elongation Factor1 (EF1) intron (Eucgr.J01112; SEQ ID NO: 27) and terminated with NOS was cloned upstream to the nptII cassette in the modified pBI121 vector using a FseI site.
(iv) A second synthetic DNA expression cassette of Bt-cry1Ab gene controlled by CaMV 35S promoter, fused to the 5′ untranslated region (5′UTR) 0 sequence of the Tobacco mosaic virus (TMV) and terminated with NOS was cloned downstream to the nptII cassette using EcoRI site.
(v) A third synthetic DNA expression cassette of Bt-cry1Bb gene controlled by the Eucalyptus Rubisco promoter Eucgr.J01502.2, fused to the 5′ untranslated region (5′UTR) of RbcS) and terminated with the 3′ UTR and terminator of RbcS was cloned downstream to the Bt-Cry1Ab cassette using an AsiSI site.

To encode Bt-Cry2Aa Bt PP (SEQ ID NO: 1), Bt-Cry1 Ab (SEQ ID NO: 3) and Bt-Cry1Bb (SEQ ID NO: 5), the nucleotide sequences SEQ ID NO: 2; SEQ ID NO: 4 and SEQ ID NO: 6, respectively, were used. The full sequence of the Triple-Bt PP binary expression vector construct is provided as SEQ ID NO: 7.

The transformation of plants with the combination of multiple Bt PP, the regeneration and selection of plants were performed essentially as described in Example 4 above.

The resulting Eucalyptus transgenic events expressing the Bt PP Cry2Aa (SEQ ID NO: 1), Cry1Ab (SEQ ID NO: 3) and Cry1Bb (SEQ ID NO: 5) are described herein and below as Triple-Bt-Tg.

### Example 7: Western Blot Verifies Expression of Triple-Bt-Tg

In order to verify that Triple-Bt-Tg plants have intact protein-level expression of the Cry2Aa, Cry1Ab and Cry1Bb Bt PP, we extracted crude protein samples from plant tissue and performed Western blot using Bt-specific antibodies.

For protein extraction, 100 mg of a Triple-Bt-Tg cultured shoot tissue (from Tg event No. 38) and control clone cultured shoot tissue (AEC0224, a background clone) were ground with 10 mg PVPP using a bead-beater and suspended in 500 μl extraction buffer (30 mM Hepes-NaOH pH 7.5, 0.5M NaCl, 2% Triton x-100 and 2 μl/ml Protease inhibitor cocktail). Samples were then incubated on ice for an hour and then centrifuged for 15 minutes at 13,000 RPM, 4° C. Supernatant was collected and added with 1:1 sample buffer.

For Western blot analysis, SDS-PAGE was performed using a Bolt™ 4-12% Bis-Tris Plus gel (Thermo Scientific, NW04120BOX) in a Mini Gel Tank apparatus containing running buffer (Thermo Scientific, #B0002). Protein size standard (Page Ruler, pre-stained protein ladder, Thermo Scientific. 26616) was included on the gel. After 40 minutes of 160V electrophoresis, proteins were transferred to a cellulose nitrate membrane (Whatman, Protran BA-85, 0.45 μm, 10401130) using a Trans-Blot semi dry transfer (Bio-Rad, Israel). Membrane was blocked with 5% skim milk powder in Phosphate-Buffered Saline (PBSX1; Biological Industries, 02-023-5A) for 1 hour. The membrane was incubated overnight and probed with the appropriate antibody, as described below.

For identification of Cry2Aa, 20 μl of sample were loaded in each well and the membrane was probed with a custom Cry2Aa polyclonal antibody diluted 1:1000. The antibody was produced by GenScript (https://www.genscript.com/) and raised in rabbit against the peptide of domain #2 in the Cry2Aa protein. For identification of Cry1Bb 20 μl of sample were loaded in each well and the membrane was probed with a custom Cry1Bb polyclonal antibody diluted 1:500. The antibody was produced by GenScript (genscript.com/on the World Wide Web) and raised in rabbit against the peptide of domain #3 in the Cry1Bb protein. For identification of Cry1Ab, 40 μl of sample was loaded in each well and the membrane was probed with a monoclonal anti-Cry1Ab antibody (MyBioSource: MBS857773) diluted 1:250.

For each of the Bt PP assayed, the membrane was washed the following day 3 times in PBS×1 and incubated with the secondary antibody HRP-conjugated goat anti-rabbit IgG antibody (Sigma. #A0545) or anti-mouse IgG antibody (Sigma, #A9917), diluted 1:10000 in PBS×1 as required. The membrane was subsequently washed 3 times with PBS×1 for 5 minutes. The signal was visualized using ECL reagent (WesternBright ECL Western blotting detection kit, Advansta, K-12043-D20) and the images were developed using the imager Omega Lum G (Aplegen).

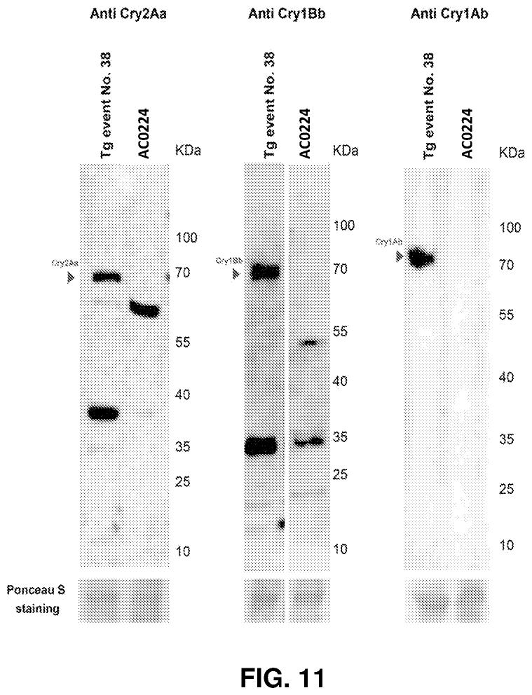

In the Triple-Bt-Tg derived sample all three Bt PP (i.e., Cry1Ab, Cry1Bb and Cry2Aa), had an observed prominent band, approximately 70 KDa, in accordance with the monoisotopic calculated molecular weights, 69.7 KDa. 73.9 KDa, and 70.8 KDa, respectively. In contrast, no bands were observed near the predicted MW, 70 KDa, in AEC0224 controls (FIG. 11; ponceau S staining is provided below as control for loading amounts on the gel).

### Example 8: Resistance Evaluation of Bt PP Expressing Triple-Transgenic Plants (Triple-Bt-Tg) from Field-Trials

In order to assess potency of the specific BT-triple combination (i.e., Cry1Ab, Cry1Bb and Cry2Aa; Triple-Bt-Tg) in protecting Eucalyptus plants grown in the field, a percent damage quantification was used.

Specifically leaves taken periodically over the course of up to twelve months from plants grown in field conditions, and were assayed in the lab (leaf slices placed on Whatman filter paper 1 mm, as above in Example 3). The percent surface damage per leaf was assessed (as in Example 5 above) and categorized as follows: plants having less than 5% damage were categorized as resilient (resistant), plants having between 5% and 10% damage were categorized as having medium resistance and plants having 11% or more damage were defined as susceptible. Each event was assessed using 10 T. arnobia caterpillars for 7 days. Percentages were rounded to the closest whole number.

Samples from Triple-Bt-Tg plants were assessed alongside tissues derived from 4 non-transgenic clones, specifically: (1) AEC0224 (2) AC0144 and (3) BA1175, which served as background clones for the transgenic insertions of the plants above; as well as (4) clone ITA25. These 4 control clones were grown side-by-side with the Triple-Bt-Tg plants.

Table 7 summarizes the above-described lab-experiments performed using material from plants grown over the course of several months in the field. All 32 transgenic events reported in Table 7 below are Triple-Bt-Tg.

TABLE 7
Leaf Damage quantification due to herbivorous caterpillar activity (% damage to leaf)

| Event | January | February | March | April | May | June | July | September | November | December |
|---|---|---|---|---|---|---|---|---|---|---|
| Tg event No. 21 | 0% | 4% | — | — | — | — | — | — | — | — |
| Tg event No. 22 | 0% | 2% | 2% | 4% | 13% | — | — | — | — | — |
| Tg event No. 23 | 33% | 18% | — | — | — | — | — | — | — | — |
| Tg event No. 24 | 5% | 17% | — | — | — | — | — | — | — | — |
| Tg event No. 25 | 1% | 10% | 2% | 3% | 3% | 0 | 0 | — | — | — |
| Tg event No. 26 | 2% | 6% | — | — | — | — | — | — | — | — |
| Tg event No. 27 | 5% | 4% | 5% | 10% | — | — | — | — | — | — |
| Tg event No. 28 | 1% | 3% | — | — | — | — | — | — | — | — |
| Tg event No. 29 | 4% | 4% | — | — | — | — | — | — | — | — |
| Tg event No. 30 | 4% | 5% | 3% | 6% | 8% | — | — | — | — | — |
| Tg event No. 31 | 5% | 8% | 3% | 9% | — | — | — | — | — | — |
| Tg event No. 32 | 1% | 18% | — | — | — | — | — | — | — | — |
| Tg event No. 33 | 0% | 2% | 2% | 2% | 3% | <1% | <1% | — | — | — |
| Tg event No. 34 | 4% | 3% | 2% | 3% | 4% | — | — | — | — | — |
| Tg event No. 35 | 5% | 3% | 2% | 3% | 3% | — | — | — | — | — |
| Tg event No. 36 | 4% | 16% | — | — | — | — | — | — | — | — |
| Tg event No. 37 | 0% | 5% | 1% | 2% | 2% | — | — | — | — | — |
| Tg event No. 38 | 1% | 3% | 1% | 1% | 2% | 0% | 0% | 0% | 0% | 0% |
| Tg event No. 39 | 0% | 4% | — | — | — | — | — | — | — | — |
| Tg event No. 40 | 8% | 15% | 2% | 4% | 10% | 0 | 0 | — | — | — |
| Tg event No. 41 | 3% | 2% | 1% | 1% | — | — | — | — | — | — |
| Tg event No. 42 | 0% | 8% | 1% | 3% | 5% | — | — | — | — | — |
| Tg event No. 43 | 0% | 4% | 2% | 3% | 5% | — | — | — | — | — |
| Tg event No. 44 | 2% | 3% | 2% | 3% | 8% | — | — | — | — | — |
| Tg event No. 45 | 4% | 5% | 2% | 4% | 11% | — | — | — | — | — |
| Tg event No. 46 | 2% | 6% | 2% | 3% | 4% | — | — | — | — | — |
| Tg event No. 47 | 0% | 10% | 2% | 4% | 3% | <1% | <1% | — | — | — |
| Tg event No. 48 | 45% | 36% | — | — | 44% | >10% | >10% | >10% | 58% | 52% |
| Tg event No. 49 | 3% | 3% | 1% | 1% | 2% | 0% | 0% | 0% | 0% | 0% |
| Tg event No. 50 | 0% | 7% | 3% | 4% | 4% | — | — | — | — | — |
| Tg event No. 51 | 0% | 2% | 2% | 2% | 4% | <1% | <1% | — | — | — |
| Non-Tg AEC0224 | 75% | 73% | 42% | 80% | 62% | >20% | >20% | — | — | — |
| Non-Tg AC0144 | 50% | 53% | 47% | 83% | 74% | >20% | >20% | — | — | — |
| Non-Tg BA1175 | 60% | 58% | 58% | 90% | 82% | >20% | >20% | — | — | — |
| Non-Tg ITA25 | 85% | 26% | 56% | 76% | 83% | >20% | >20% | >20% | 67% | 24% |

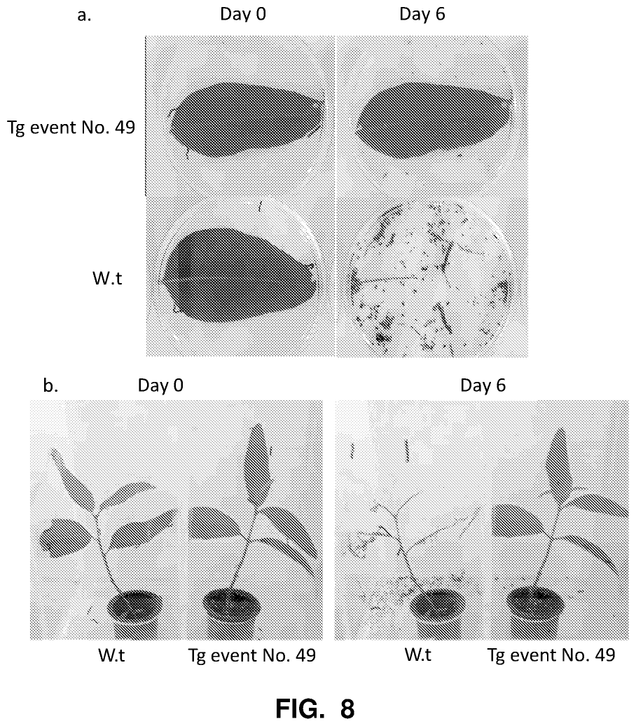

The Triple-Bt-Tg events Tg event No. 38 and Tg event No. 49 demonstrated the least amount of leaf damage and maximum resistance, in these studies, and are marked in Table 7 above in gray, for clarity. For example, leaf or seedling damage to Wild Type (W.t) plants compared to Triple-Bt-Tg (Tg) event No. 49 plants can be seen in FIG. 8. Leaf damage (FIG. 8a, upper panel) or seedling damage (FIG. 8b, lower panel) inoculated with 3 or 10 2^{nd} instar T. arnobia caterpillar larvae on leaves and seedlings, respectively, observed at time 0 and after 6 days. The Wt seedling and leaf were totally consumed after 6 days while Triple-Bt-Tg No. 49 seedling and leaf showed only insignificant damage.

With respect to these two events, although some minor leaf-damage was observed, this was consistent with leaf digestion leading to rapid caterpillar death and, furthermore, always showed 100% mortality of caterpillars overtime. Worthy of note: in regard to the two leading events, Tg Events 38 and 49, we did not observe any adverse effects on agronomic qualities, such as plant growth.

### Example 9: Lyophilized Tissue from Triple-Bt-Tg Eucalyptus Plants is Toxic to T. arnobia when Incorporated in Artificial Diet, Even after Substantial Dilution

To quantitatively address the potency of the transgenic expressed Triple Bt PP combination, we exposed caterpillars to dried leaf material from Triple-Bt-Tg or to dried leaf material from non-transgenic plants, mixed in different dilution-ratios into artificial diet.

Lyophilized (freeze-dried) leaf material from Triple-Bt-Tg Event No. 49 (as well as from ITA25 and AEC0224 non Tg control background plants) was incorporated into artificial diet at various concentrations, as described below. For lyophilization, leaves collected were placed in a container connected to an Edwars lyophilizing vacuum pump and kept in vacuum for 72 hours until the material was completely dry.

Lyophilized leaves were then crushed using a blender until a fine powder constituency was achieved. Dilutions in different proportions were obtained by mixing powder into artificial diet. The calculated fold dilution is the weight (gr) of leaves (before they were lyophilized)/weight (gr) of artificial diet. Treatments and dilutions of test items are summarized in Table 8 below.

TABLE 8
Values for calculation of the applied
dilution (artificial diet/fresh weight)

| Dilution | Grams of Fresh | Grams of | Grams of Lyophilized |
|---|---|---|---|
| Treatment | Weight | Artificial Diet | leaf tissue |
| 2000 | 0.075 | 150 | 0.021 |
| 1600 | 0.094 | 150 | 0.026 |
| 1200 | 0.125 | 150 | 0.035 |
| 800 | 0.188 | 150 | 0.05 |
| 400 | 0.375 | 150 | 0.11 |
| 350 | 0.43 | 150 | 0.12 |
| 300 | 0.5 | 150 | 0.14 |
| 250 | 0.6 | 150 | 0.17 |
| 200 | 0.75 | 150 | 0.21 |
| 100 | 1.5 | 150 | 0.42 |
| 87.5 | 1.72 | 150 | 0.48 |
| 75 | 2 | 150 | 0.56 |
| 62.5 | 2.4 | 150 | 0.67 |
| 50 | 3 | 150 | 0.84 |
| 37.5 | 4 | 150 | 1.12 |
| 25 | 6 | 150 | 1.68 |
| 12.5 | 12 | 150 | 3.36 |
| 6.25 | 24 | 150 | 6.72 |

After incorporation, each treatment (leaf+artificial diet) was divided into 20 glass tubes (5 mL per tube), and one caterpillar was added per tube, to produce 20 repetitions per treatment in total.

Each plant-derived sample was “diluted” 1:6.25 fold; 1:12.5 fold; 1:25 fold; 1:37.5 fold; 1:50 fold; 1:100 fold; 1:200 fold; 1:250 fold; 1:300 fold; 1:350 fold; 1:400 fold; 1:800 fold; 1:1200 fold; 1:1600 and 1:2000 fold. In effect, for example, 1:6.25-fold means that lyophilized leaf powder from X gr leaves was mixed with 6.25*X (gr) artificial diet. The different sample-dilutions are referred to below as ‘Dilution treatment’. The artificial diet in the current example and as default throughout this disclosure was detailed in Wilckenand and Berti Filho, 2006, and made as provided in the methods section above.

Plant leaf dilution mixes, prepared as above, were divided into 20 glass tube replicates: each with a volume of 5 mL leaf artificial diet mixture per tube to which a single caterpillar was introduced (20 biological repetitions per treatment). Positive Controls were prepared by supplementing artificial diet with DiPel® as follows:

1 mL of DiPel® Commercial stock (obtained from Sumitomo Chemical) was diluted in 1000 mL of water. 0.75 ml of diluted DiPel® (1:1000) was added to 5 ml artificial diet to serve as positive control. 20 repetitions, 1 caterpillar per tube were performed for positive control. Negative (Diet only) controls were prepared by adding 5 ml artificial diet without any additional compounds, 20 repetitions (1 caterpillar each). Percent mortality was assessed at the end of the 7th day from commencement of the experiment. Table 9 below provides the percent mortality of caterpillars after 7 days of exposure to lyophilized leaves. AEC0224 is the untransformed background clone of the Tg event No. 49 Triple-Bt-Tg line.

TABLE 9
Percent mortality of caterpillars after 7 days of exposure
to lyophilized leaves added to artificial diet

| | Triple-Bt Tg | | | | |
|---|---|---|---|---|---|
| Treatment/ | Tg event No. | | | Positive | Negative |
| Dilution | 49 | AEC0224 | ITA25 | Control | Control |
| 1:6.25 | 100 | 20 | 20 | — | — |
| 1:12.5 | 100 | 10 | 10 | — | — |
| 1:25 | 100 | 60 | 10 | — | — |
| 1:37.5 | 100 | 50 | 20 | — | — |
| 1:50 | 100 | 0 | 20 | — | — |
| 1:100 | 65 | 20 | — | — | — |
| 1:200 | 55 | 5 | — | — | — |
| 1:250 | 30 | 20 | — | — | — |
| 1:300 | 50 | 5 | — | — | — |
| 1:350 | 40 | 10 | — | — | — |
| 1:400 | 50 | 25 | — | — | — |
| 1:800 | 15 | 5 | — | — | — |
| 1:1200 | 40 | 20 | — | — | — |
| 1:1600 | 10 | 15 | — | — | — |
| 1:2000 | 0 | 5 | — | — | — |
| Artificial | — | — | — | — | 10 |
| Diet (AD) only | | | | | |
| DiPel ®/AD | — | — | — | 100 | — |

DiPel ® positive control treatment showed 100% mortality whereas the negative control of artificial-diet-only showed 10% mortality.

Freeze-dried leaves of the Triple-Bt Tg event No. 49 harboring the Bt PP combination of Cry1Bb Cry2Aa and Cry1Ab polypeptides lead to killing of 100% of all tested caterpillars, at dilution levels up to 1:50× dilution, by the day 7 endpoint. At higher dilutions, up 1:400, Triple-Bt Tg event No. 49 added to artificial diet, resulted caterpillar mortality rate higher than control AEC0224.

### Example 10: Toxicity of Plant Tissue Derived from Field Trial Transgenic Eucalyptus Plants Expressing Triple Bt PP to Physocleora dukinfeldia

The efficacy of the Triple-Bt-Tg genetic construct (consisting of the Cry1Ab, Cry1Bb, and Cry2Aa genes) in conferring resistance to eucalyptus plants grown under field conditions for two years was evaluated through the quantification of the percentage of mortality of Physocleora dukinfeldia when exposed to Eucalyptus Plants Expressing Triple Bt PP. Leaf samples were collected from field-grown plants and subsequently subjected to laboratory analysis. Leaves were prepared and placed on Whatman filter paper (1 mm thickness) like that described previously in Example 3. The assay was conducted using 10 Physocleora dukinfeldia larvae per sample for a period of 7 days. The percentages were rounded to the nearest whole number. Comparison samples were also obtained from 4 non-transgenic clones (AEC0224, AC0144, BA1175, and ITA25) that were grown alongside the Triple-Bt-Tg plants. The results of these laboratory experiments are summarized in Table 10, which includes data from 4 transgenic events of the Triple-Bt-Tg construct.

TABLE 10
Percent Mortality

| | Percent mortality after |
|---|---|
| Source | 7 days |
| Tg event No. 49 | 100% |
| Tg event No. 55 | 100% |
| Tg event No. 56 | 100% |
| Tg event No. 57 | 100% |
| Non Tg control: | 53% |
| (BA1175) | |
| Non Tg control: | 33% |
| (BA1338) | |
| Non Tg control: | 20% |
| (AEC0224) | |
| Non Tg control: | 13% |
| (BA1175) | |
| Non Tg control: | 0% |
| (Ita 25) | |

### Example 11: Effectiveness of Genetically Modified Eucalyptus (Tg Event No. 49) in Controlling Thyrinteina Arnobia (Lepidoptera: Geometridae) Under Field Conditions

Evaluating the effectiveness of control of GM eucalyptus (Tg event no. 49) against T. arnobia caterpillar, under controlled conditions in the field in representative regions of eucalyptus cultivation in Brazil.

### 1. Material and Methods

Trials were conducted in 3 locations, Angatuba/SP, Ibaté/SP and Tres Lagoas/MS. The trials consisted of 3 treatments: eucalyptus Tg event no. 49 (TR01), eucalyptus FGN-K (TR02) without pesticide application and eucalyptus FGN-K (TR03) with pesticide application (Dipel), arranged in 5 random blocks with linear plots of 6 plants with spacing of 3.0×3.0M. FGN-K is the AEC0224 wildtype clone.

No pesticide applications were made in the experimental area, except for the FGN-K+Dipel (TR03) treatment plots, during the experiment.

The caterpillars used in the experiments were from the stock breeding maintained in the Entomology Laboratory of Suzano S. A. (FuturaGene—Biotechnology Division.

Before the release in the field, the individual viability of each caterpillar was checked and ensured, and, when necessary, replacement of compromised caterpillars was made.

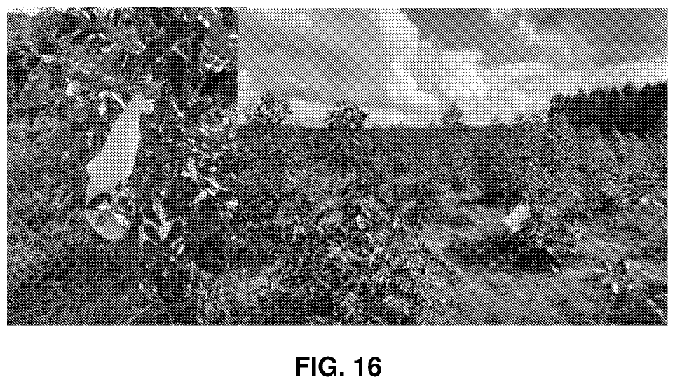

For exposure to the treatments and consumption by the caterpillars, cages were used made of “voil”, a thin cloth, with a side opening and a Velcro system and an opening at the base for the introduction of the branch (FIG. 16). The cages allowed for natural positioning of the branch without forcing its tip down. The branches used were covered with the cage that had its base sealed with cord and tape, preventing the entry or exit of insects. Prior to the isolation of the branches and release of the caterpillars in the cages, an inspection and cleaning process was performed, removing the presence of predators inside the cages.

In each of the 5 blocks of the trial, one plant was selected for each of the 3 treatments, totaling 5 repetitions per treatment. A branch from the lower third of each plot plant was selected, avoiding branches that had necrotic areas, damage, or other injuries (FIG. 16).

On the selected branch, inside the cage, 30 caterpillars, with a maximum age of 24 hours, were added, allowing for the exposure and consumption of the leaves by Thyrinteina arnobia caterpillars.

The methodological procedure was strictly adhered to in each of the three locations, avoiding variations in the methodology applied in each experiment, restricting variability only to those inherent to the location of each trial, such as temperature, humidity, photoperiod, among others.

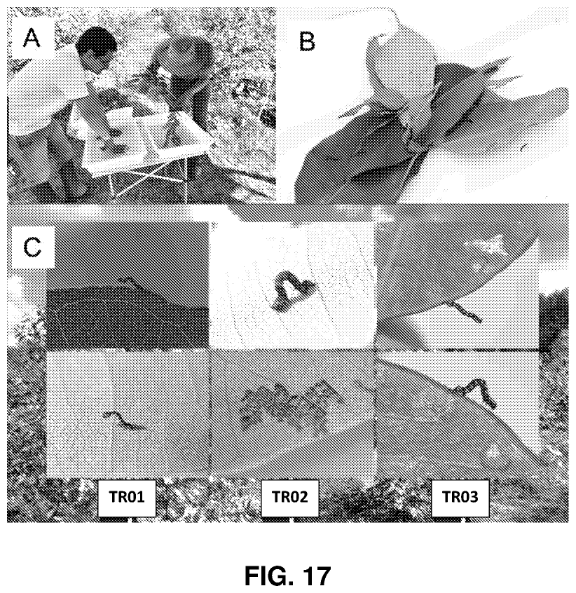

The caterpillars in each treatment were allowed to grow for 7 days at which time the cages were opened and the final evaluation of the number of dead insects, out of the total initially released insects, was carried out (FIG. 17).

a) Results from Angatuba/SP

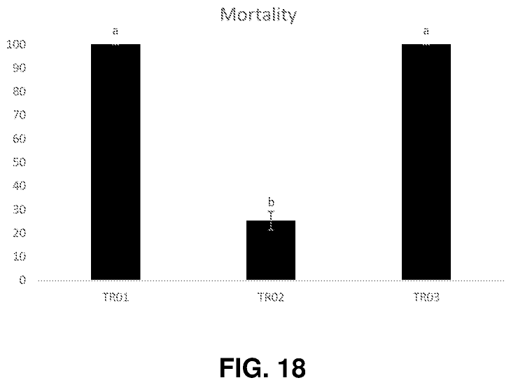

The FGN-K (TR03) treatment with insecticide application, positive control, resulted in 100% mortality and did not differ statistically from the results found with the Tg event no. 49 (TR01) treatment, which presented 100% mortality as well (FIG. 18). The FGN-K treatment (TR02) without insecticide application had a survival rate of 74.7%, statistically different from the other treatments. The high survival rate observed in the treatment without insecticide demonstrates that the methodology used was effective in allowing the survival and development of the caterpillars in the cages under experimental conditions in the field, indicating that the exposure to the treatments TR01 and TR03, the application of insecticide and Tg event no. 49, respectively, were effective in controlling growth of Thyrinteina arnobia caterpillars under experimental field conditions.

b) Tg Event No. 49 Results from Ibaté/SP

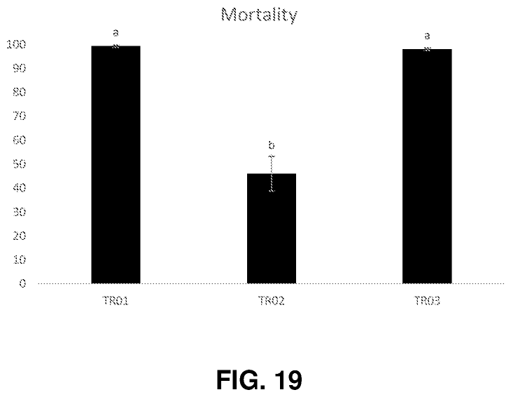

The Tg event no. 49 (TR01) treatment showed an efficiency of 99.3% in controlling Thyrinteina arnobia caterpillars, being statistically equal to the FGN-K treatment (TR03) with insecticide application that showed a mortality of 98.0%, both significantly different from the FGN-K treatment (TR02) without insecticide application (FIG. 19) which showed a 54% survival rate. The results demonstrated that the methodology used in eucalyptus Tg event no. 49 confers resistance against caterpillars of the species T. arnobia in 1st instar.

c) Results from Tres Lagoas/MS

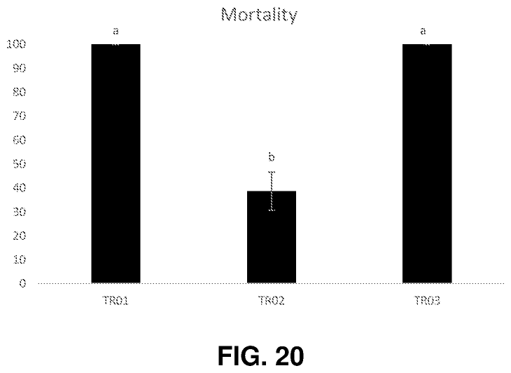

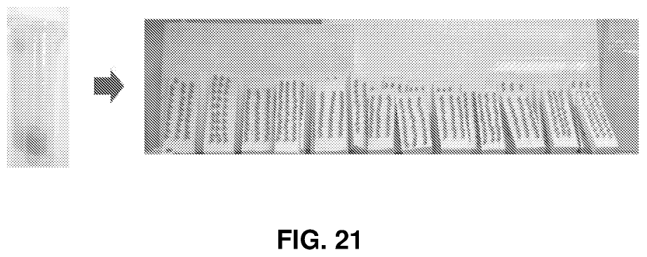

The Tg event no. 49 (TR01) treatment showed an efficiency of 99.3% in controlling Thyrinteina arnobia caterpillars, being statistically equal to the FGN-K treatment (TR03) with insecticide application that showed a mortality of 98.0%, both significantly different from the FGN-K treatment (TR02) without insecticide application (FIG. 20) which showed 54% survival rate. The results demonstrated that the methodology used in eucalyptus Tg event no. 49 confers resistance against caterpillars of the species T. arnobia in 1st instar.

In the 3 (three) locations, the results were similar and consistent, with the Tg event no. 49 (TR01) treatment statistically differing from the FGN-K treatment (TR02) without insecticide application, but with a similar response to the FGN-K treatment (TR03) with insecticide application.

The similarity between the results of experiments conducted in different locations allows us to conclude that the ability to control 1st (first) instar caterpillars of the species T. arnobia by using Tg event no. 49 was consistent in different environments. This response stability is important to meet the objective of selecting commercial eucalyptus genotypes, tolerant to abiotic stresses, as well as to biotic stress, such as those caused by pests and diseases.

### Example 12: Generating Transgenic Eucalyptus Plants Expressing a Triple Bt PP by Crossing Transgenic Plants Expressing Different Bt PP

In order to generate further plants resistant to insect pests by expressing a Triple Bt PP of Bt-Cry2Aa, Bt-Cry1Ab and Bt-Cry1Bb: transgenic plants such as those described in Example 2 above can be crossed, by conventional breeding methods, to generate progenies carrying constructs encoding double or triple-transgenic Bt PP (and thereby expressing double or triple-transgenic Bt PP) which can be further verified using genomic or phenotype testing methods.

Methods for crossing plants to obtain multiple transgenic events are known in the art. Specifically, transformation of separate single-Bt PP constructs (as in Example 1 above) into plants, regeneration, and selection can be performed as described in detail above in Example 2, or as is known in the art. The resulting transgenic events expressing single Bt PP can be verified for having intact genomic integration by PCR, sequencing and genomic methods or phenotype testing, and/or Bt PP protein expression level verified, such as by western blot, as in Example 7 above. Such plants expressing a single Bt PP can be crossed with other plants expressing a single, double or more Bt PP to produce offspring expressing more than one Bt PP.

For example, steps in the crossing process may include the following and are not limited to a particular order nor pollen donor and/or the female flowering plant:
1. Pollen from the selected event carrying the cry1Ab gene may be collected and used to fertilize a flower of a selected event carrying cry2Aa gene, for example.
2. Seeds generated by this fertilization may be collected and germinated in the nursery to produce seedlings whereas a leaf sample of may be excised and tested by PCR to confirm the presence of both cry1Ab and cry2Aa genes and/or a Western Blot can be performed to confirm the presence of both Cry1Ab and Cry2Aa proteins.
3. Pollen from the selected event carrying the cry1Bb gene may be collected and used to fertilize a flower of a selected event carrying both cry1Ab and cry2Aa genes.
4. Seeds generated by this fertilization may be collected and germinated in the nursery to produce seedlings whereas a leaf sample of may be excised and tested by PCR to confirm the presence of cry1Ab, cry2Aa and cry/Bb genes and/or a Western Blot can be performed to confirm the presence of Cry1Ab, Cry2Aa and Cry1Bb proteins.
5. The triple Bt PP gene confirmed event can be tested in the greenhouse and in the field, as described above.

Once breeders identify the desired genetically modified plant emanating from a specific transgenic event, a clone, and have further confirmed its resistance to a target insect pest, such as Thyrinteina arnobia and Physocleora spp, they may proceed to mass propagate this clone to produce large numbers of clones (clonal material) prior to large scale planting. This may be performed through tissue culture practices or through coppicing followed by shoot growth, collection of cuttings and rooting of cuttings, prior to replanting. Clone production from a specific transgenic event, results in the production of clones which contain identical genetic material to that of the original transgenic event.

Breeders can also generate genetically modified clones by crossing a single transgenic event expressing the double or triple Bt PP of Bt-Cry2Aa, Bt-Cry1Ab and Bt-Cry1Bb, with conventional wild type parent genotypes from the classical breeding program or with transgenic phenotypes expressing other transgenes of interest. The resulting transgenic clones expressing the double or triple Bt PP can be verified for having intact genomic integration by PCR, sequencing and genomic methods or phenotype testing, and/or Bt PP protein expression level verified, such as by western blot, as in Example 7 above.

### Example 13: Quantification of Cry2Aa, Cry1Ab, Cry1Bb and NPTII Proteins Using the Enzyme-Linked Immunosorbent Assay (ELISA) Methodology in Eucalyptus Tissues, Tg Event No. 49

### 1. Objective

This study aimed to quantify proteins Cry2Aa, Cry1Ab, Cry1Bb and NPTII in young leaves, mature leaves, branches, roots, flower buds and pollen of genetically modified eucalyptus Tg event no. 49, using ELISA (Enzyme-Linked Immunosorbent Assay) methodology.

### 2. Test System

The Test Systems for this Study are the tissues of genetically modified eucalyptus plants and tissues of conventional wild type clones (young leaf, mature leaf, branch, root, flower bud and pollen). The plants used to collect the Test System were obtained from experiments installed at two different locations Farm 1 (SP) and Farm 2 (SP).

### 3. Test Items

The Test Items are the proteins: Cry2Aa, Cry1Ab, Cry1Bb and NPTII.

### 4. Reference Items

The Reference Items of this Study are the proteins Cry1Ab, Cry1Bb and Cry2Aa, produced on demand by the company Fraunhofer-Gesellschaft.

### Technical Specifications of the Reference Items:

### Cry1Ab Protein

Manufacturer: Fraunhofer-Gesellschaft
Lot: EXP-22AC5753
Host: Pseudomonas Fluorescens
Purity: >80% by densitometry
Storage: store at −80° C.
Sequence size: 621 aa
Molecular weight: 69.6 kDa
Amino acid sequence: SEQ ID NO:45

This sequence has minor changes in the N-terminal region (relative to SEQ ID NO:3) to protect this region of the expressed protein from trypsin digestion during recombinant production. The core active three domains are the same as in the transgenic eucalyptus. Activity of this expressed protein has been verified.

### Cry1Bb Protein

Manufacturer: Fraunhofer-Gesellschaft
Lot: EXP-22AC3967
Host: Pseudomonas fluorescens
Purity: >35% by densitometry
Storage: store at −80° C.
Sequence size: 655 aa
Molecular weight: 73.99 kDa
Amino acid sequence: SEQ ID NO: 46

This sequence has minor changes in the N-terminal region (relative to SEQ ID NO:5) to protect this region of the expressed protein from trypsin digestion during recombinant production. The core active three domains are the same as in the transgenic eucalyptus. Activity of this expressed protein has been verified.

### Cry2Aa Protein

Manufacturer: Fraunhofer-Gesellschaft
Lot: EXP-22AC9418
Host: Pseudomonas fluorescens
Purity: >90% by densitometry
Storage: store at −80° C.
Sequence size: 639 aa
Molecular weight: 71.7 kDa
Amino acid sequence: SEQ ID NO: 47

This protein has C-terminal His-Tag but is otherwise the same as SEQ ID NO:1). The core active three domains are the same as in the transgenic eucalyptus. Activity of this expressed protein has been verified.

### 5. Methodology and Evaluation of Results

### 5.1. Field Phase (Sample Collection)

### 5.1.1. Origin of Genetic Material

The samples, of the Tg event no. 49 and of the conventional wild type clone FGN-K, comprising young leaves, mature leaves, branches and roots were collected 6 months, 12 months and 24 months after planting, in Farm 1 and Farm 2 (Table 11).

Upon the first flowering event in the field, samples of Tg event no. 49 and the conventional clone FGN-K, of flower bud and pollen, were collected

At Farm 1, the eucalyptus seedlings were planted on Oct. 8, 2019 in plots of 16 plants, 4 plants in each row, with 4 rows. The planting spacing adopted was 3.0×2.0 m.

At Farm 2, the eucalyptus seedlings were planted on Nov. 13, 2019 in plots of 16 plants, 4 plants in each row, with 4 rows. The planting spacing adopted was 3.0×2.5 m.

The trial with a randomized block design was composed of 5 treatments and 5 blocks (replications). The experimental plots were protected by 2 conventional border eucalyptus lines in all four directions.

TABLE 11
Description of samples received for quantification by
ELISA of proteins Cry1Ab, Cry2Aa, Cry1Bb and NPTII.

| Samples | Identification | Tissue | Age | | Collection | Delivery Date |
|---|---|---|---|---|---|---|
| Tg event no. 49 | BL01 TR02 - Yl | Young Leaves | 6 | months | Farm 1 | Apr. 23, 2020 |
| Tg event no. 49 | BL01 TR02 - Ml | Mature Leaves | 6 | months | Farm 1 | Apr. 23, 2020 |
| Tg event no. 49 | BL01 TR02 - St | Stem | 6 | months | Farm 1 | Apr. 23, 2020 |
| Tg event no. 49 | BL01 TR02 - Rt | Root | 6 | months | Farm 1 | Apr. 23, 2020 |
| Tg event no. 49 | BL02 TR02 - Yl | Young Leaves | 6 | months | Farm 1 | Apr. 23, 2020 |
| Tg event no. 49 | BL02 TR02 - Ml | Mature Leaves | 6 | months | Farm 1 | Apr. 23, 2020 |
| Tg event no. 49 | BL02 TR02 - St | Stem | 6 | months | Farm 1 | Apr. 23, 2020 |
| Tg event no. 49 | BL02 TR02 - Rt | Root | 6 | months | Farm 1 | Apr. 23, 2020 |
| Tg event no. 49 | BL03 TR02 - Yl | Young Leaves | 6 | months | Farm 1 | Apr. 23, 2020 |
| Tg event no. 49 | BL03 TR02 - Ml | Mature Leaves | 6 | months | Farm 1 | Apr. 23, 2020 |
| Tg event no. 49 | BL03 TR02 - St | Stem | 6 | months | Farm 1 | Apr. 23, 2020 |
| Tg event no. 49 | BL03 TR02 - Rt | Root | 6 | months | Farm 1 | Apr. 23, 2020 |
| FGN-K | BL03 TR03 - Yl | Young Leaves | 6 | months | Farm 1 | Apr. 23, 2020 |
| FGN-K | BL03 TR03 - Ml | Mature Leaves | 6 | months | Farm 1 | Apr. 23, 2020 |
| FGN-K | BL03 TR03 - St | Stem | 6 | months | Farm 1 | Apr. 23, 2020 |
| FGN-K | BL03 TR03 - Rt | Root | 6 | months | Farm 1 | Apr. 23, 2020 |
| Tg event no. 49 | BL01 TR02 - Yl | Young Leaves | 12 | months | Farm 1 | Oct. 21, 2020 |
| Tg event no. 49 | BL01 TR02 - Ml | Mature Leaves | 12 | months | Farm 1 | Oct. 21, 2020 |
| Tg event no. 49 | BL01 TR02 - St | Stem | 12 | months | Farm 1 | Oct. 21, 2020 |
| Tg event no. 49 | BL01 TR02 - Rt | Root | 12 | months | Farm 1 | Oct. 21, 2020 |
| Tg event no. 49 | BL02 TR02 - Yl | Young Leaves | 12 | months | Farm 1 | Oct. 21, 2020 |
| Tg event no. 49 | BL02 TR02 - Ml | Mature Leaves | 12 | months | Farm 1 | Oct. 21, 2020 |
| Tg event no. 49 | BL02 TR02 - St | Stem | 12 | months | Farm 1 | Oct. 21, 2020 |
| Tg event no. 49 | BL02 TR02 - Rt | Root | 12 | months | Farm 1 | Oct. 21, 2020 |
| Tg event no. 49 | BL03 TR02 - Yl | Young Leaves | 12 | months | Farm 1 | Oct. 21, 2020 |
| Tg event no. 49 | BL03 TR02 - Ml | Root | 12 | months | Farm 1 | Oct. 21, 2020 |
| Tg event no. 49 | BL03 TR02 - St | Mature Leaves | 12 | months | Farm 1 | Oct. 21, 2020 |
| Tg event no. 49 | BL03 TR02 - Rt | Stem | 12 | months | Farm 1 | Oct. 21, 2020 |
| FGN-K | BL03 TR03 - Yl | Young Leaves | 12 | months | Farm 1 | Oct. 21, 2020 |
| FGN-K | BL03 TR03 - Ml | Mature Leaves | 12 | months | Farm 1 | Oct. 21, 2020 |
| FGN-K | BL03 TR03 - St | Stem | 12 | months | Farm 1 | Oct. 21, 2020 |
| FGN-K | BL03 TR03 - Rt | Root | 12 | months | Farm 1 | Oct. 21, 2020 |
| Tg event no. 49 | BL01 TR02 - Yl | Young Leaves | 24 | months | Farm 1 | Oct. 6, 2021 |
| Tg event no. 49 | BL01 TR02 - Ml | Mature Leaves | 24 | months | Farm 1 | Oct. 6, 2021 |
| Tg event no. 49 | BL01 TR02 - St | Stem | 24 | months | Farm 1 | Oct. 6, 2021 |
| Tg event no. 49 | BL01 TR02 - Rt | Root | 24 | months | Farm 1 | Oct. 6, 2021 |
| Tg event no. 49 | BL02 TR02 - Yl | Young Leaves | 24 | months | Farm 1 | Oct. 6, 2021 |
| Tg event no. 49 | BL02 TR02 - Ml | Mature Leaves | 24 | months | Farm 1 | Oct. 6, 2021 |
| Tg event no. 49 | BL02 TR02 - St | Stem | 24 | months | Farm 1 | Oct. 6, 2021 |
| Tg event no. 49 | BL02 TR02 - Rt | Root | 24 | months | Farm 1 | Oct. 6, 2021 |
| Tg event no. 49 | BL03 TR02 - Yl | Young Leaves | 24 | months | Farm 1 | Oct. 6, 2021 |
| Tg event no. 49 | BL03 TR02 - Ml | Root | 24 | months | Farm 1 | Oct. 6, 2021 |
| Tg event no. 49 | BL03 TR02 - St | Mature Leaves | 24 | months | Farm 1 | Oct. 6, 2021 |
| Tg event no. 49 | BL03 TR02 - Rt | Stem | 24 | months | Farm 1 | Oct. 6, 2021 |
| FGN-K | BL03 TR03 - Yl | Young Leaves | 24 | months | Farm 1 | Oct. 6, 2021 |
| FGN-K | BL03 TR03 - Ml | Mature Leaves | 24 | months | Farm 1 | Oct. 6, 2021 |
| FGN-K | BL03 TR03 - St | Stem | 24 | months | Farm 1 | Oct. 6, 2021 |
| FGN-K | BL03 TR03 - Rt | Root | 24 | months | Farm 1 | Oct. 6, 2021 |
| Tg event no. 49 | BL01 TR02 - Yl | Young Leaves | 6 | months | Farm 2 | May 14, 2020 |
| Tg event no. 49 | BL01 TR02 - Ml | Mature Leaves | 6 | months | Farm 2 | May 14, 2020 |
| Tg event no. 49 | BL01 TR02 - St | Stem | 6 | months | Farm 2 | May 14, 2020 |
| Tg event no. 49 | BL01 TR02 - Rt | Root | 6 | months | Farm 2 | May 14, 2020 |
| Tg event no. 49 | BL02 TR02 - Yl | Young Leaves | 6 | months | Farm 2 | May 14, 2020 |
| Tg event no. 49 | BL02 TR02 - Ml | Mature Leaves | 6 | months | Farm 2 | May 14, 2020 |
| Tg event no. 49 | BL02 TR02 - St | Stem | 6 | months | Farm 2 | May 14, 2020 |
| Tg event no. 49 | BL02 TR02 - Rt | Root | 6 | months | Farm 2 | May 14, 2020 |
| Tg event no. 49 | BL03 TR02 - Yl | Young Leaves | 6 | months | Farm 2 | May 14, 2020 |
| Tg event no. 49 | BL03 TR02 - Ml | Mature Leaves | 6 | months | Farm 2 | May 14, 2020 |
| Tg event no. 49 | BL03 TR02 - St | Stem | 6 | months | Farm 2 | May 14, 2020 |
| Tg event no. 49 | BL03 TR02 - Rt | Root | 6 | months | Farm 2 | May 14, 2020 |
| FGN-K | BL03 TR03 - Yl | Young Leaves | 6 | months | Farm 2 | May 14, 2020 |
| FGN-K | BL03 TR03 - Ml | Mature Leaves | 6 | months | Farm 2 | May 14, 2020 |
| FGN-K | BL03 TR03 - St | Stem | 6 | months | Farm 2 | May 14, 2020 |
| FGN-K | BL03 TR03 - Rt | Root | 6 | months | Farm 2 | May 14, 2020 |
| Tg event no. 49 | BL01 TR02 - Yl | Young Leaves | 12 | months | Farm 2 | Oct. 29, 2020 |
| Tg event no. 49 | BL01 TR02 - Ml | Mature Leaves | 12 | months | Farm 2 | Oct. 29, 2020 |
| Tg event no. 49 | BL01 TR02 - St | Stem | 12 | months | Farm 2 | Oct. 29, 2020 |
| Tg event no. 49 | BL01 TR02 - Rt | Root | 12 | months | Farm 2 | Oct. 29, 2020 |
| Tg event no. 49 | BL02 TR02 - Yl | Young Leaves | 12 | months | Farm 2 | Oct. 29, 2020 |
| Tg event no. 49 | BL02 TR02 - Ml | Mature Leaves | 12 | months | Farm 2 | Oct. 29, 2020 |
| Tg event no. 49 | BL02 TR02 - St | Stem | 12 | months | Farm 2 | Oct. 29, 2020 |
| Tg event no. 49 | BL02 TR02 - Rt | Root | 12 | months | Farm 2 | Oct. 29, 2020 |
| Tg event no. 49 | BL03 TR02 - Yl | Young Leaves | 12 | months | Farm 2 | Oct. 29, 2020 |
| Tg event no. 49 | BL03 TR02 - Ml | Mature Leaves | 12 | months | Farm 2 | Oct. 29, 2020 |
| Tg event no. 49 | BL03 TR02 - St | Stem | 12 | months | Farm 2 | Oct. 29, 2020 |
| Tg event no. 49 | BL03 TR02 - Rt | Root | 12 | months | Farm 2 | Oct. 29, 2020 |
| FGN-K | BL03 TR03 - Yl | Young Leaves | 12 | months | Farm 2 | Oct. 29, 2020 |
| FGN-K | BL03 TR03 - Ml | Mature Leaves | 12 | months | Farm 2 | Oct. 29, 2020 |
| FGN-K | BL03 TR03 - St | Stem | 12 | months | Farm 2 | Oct. 29, 2020 |
| FGN-K | BL03 TR03 - Rt | Root | 12 | months | Farm 2 | Oct. 29, 2020 |
| Tg event no. 49 | BL01 TR02 - Yl | Young Leaves | 24 | months | Farm 2 | Oct. 19, 2021 |
| Tg event no. 49 | BL01 TR02 - Ml | Mature Leaves | 24 | months | Farm 2 | Oct. 19, 2021 |
| Tg event no. 49 | BL01 TR02 - St | Stem | 24 | months | Farm 2 | Oct. 19, 2021 |
| Tg event no. 49 | BL01 TR02 - Rt | Root | 24 | months | Farm 2 | Oct. 19, 2021 |
| Tg event no. 49 | BL02 TR02 - Yl | Young Leaves | 24 | months | Farm 2 | Oct. 19, 2021 |
| Tg event no. 49 | BL02 TR02 - Ml | Mature Leaves | 24 | months | Farm 2 | Oct. 19, 2021 |
| Tg event no. 49 | BL02 TR02 - St | Stem | 24 | months | Farm 2 | Oct. 19, 2021 |
| Tg event no. 49 | BL02 TR02 - Rt | Root | 24 | months | Farm 2 | Oct. 19, 2021 |
| Tg event no. 49 | BL03 TR02 - Yl | Young Leaves | 24 | months | Farm 2 | Oct. 19, 2021 |
| Tg event no. 49 | BL03 TR02 - Ml | Mature Leaves | 24 | months | Farm 2 | Oct. 19, 2021 |
| Tg event no. 49 | BL03 TR02 - St | Stem | 24 | months | Farm 2 | Oct. 19, 2021 |
| Tg event no. 49 | BL03 TR02 - Rt | Root | 24 | months | Farm 2 | Oct. 19, 2021 |
| FGN-K | BL03 TR03 - Yl | Young Leaves | 24 | months | Farm 2 | Oct. 19, 2021 |
| FGN-K | BL03 TR03 - Ml | Mature Leaves | 24 | months | Farm 2 | Oct. 19, 2021 |
| FGN-K | BL03 TR03 - St | Stem | 24 | months | Farm 2 | Oct. 19, 2021 |
| FGN-K | BL03 TR03 - Rt | Root | 24 | months | Farm 2 | Oct. 19, 2021 |

| | | | | | |
|---|---|---|---|---|---|
| Tg event no. 49 | BL01 TR02 - BF | Floral Bud | Flowering | Farm 1 | Feb. 24, 2022 |
| Tg event no. 49 | BL03 TR02 - BF | Floral Bud | Flowering | Farm 1 | Feb. 24, 2022 |
| Tg event no. 49 | BL05 TR02 - BF | Floral Bud | Flowering | Farm 1 | Feb. 24, 2022 |
| FGN-K | BL03 TR03 - BF | Floral Bud | Flowering | Farm 1 | Feb. 24, 2022 |
| Tg event no. 49 | BL01 TR09 - BF | Floral Bud | Flowering | Farm 1 | Feb. 25, 2022 |
| Tg event no. 49 | BL02 TR09 - BF | Floral Bud | Flowering | Farm 1 | Feb. 25, 2022 |
| Tg event no. 49 | BL04 TR09 - BF | Floral Bud | Flowering | Farm 1 | Feb. 25, 2022 |
| FGN-K | BL04 - TR36 - BF | Floral Bud | Flowering | Farm 1 | Feb. 25, 2022 |
| Tg event no. 49 | BL01 TR02 - Po | Pollen | Flowering | Farm 1 | Feb. 24, 2022 |
| Tg event no. 49 | BL03 TR02 - Po | Pollen | Flowering | Farm 1 | Feb. 24, 2022 |
| Tg event no. 49 | BL05 TR02 - Po | Pollen | Flowering | Farm 1 | Feb. 24, 2022 |
| FGN-K | BL03 TR03 - Po | Pollen | Flowering | Farm 1 | Feb. 24, 2022 |
| Tg event no. 49 | BL10 TR09 - Po | Pollen | Flowering | Farm 1 | Feb. 25, 2022 |
| FGN-K | BL05 TR36 - Po | Pollen | Flowering | Farm 1 | Feb. 25, 2022 |

TR02 treatment samples were collected in three blocks (BL01, BL02 and BL03) and only one sample from each block for each plant material, per farm. The TR03 treatment samples were collected in one block (BL03) and only one sample for each plant material, per farm (Table 11).

Floral bud and pollen samples were collected in the first flowering cycle in the field.

### 5.1.2. Plant Tissues

For the collection of plant tissues, conformed to standardized sampling procedure used to collect and Process the Eucalyptus leaves, branches and roots. The trees used in the collections in each period were identified in the field with labels.

The label used on the trees contains the study number, with all the collection information (material, time and farm).

Each sample is assigned a unique code that is used for identification and tracking.

The code is composed of:
1. Study number;
2. Abbreviated LPMA number;
3. Time after Planting;
4. Farm Code consisting of 2 digits;
5. Plot (Block, Collection Treatment and Genotype);
6. Collected tissue (where FJ=young leaf; FM=mature leaf; Rm=stem; Rz=root; BF=flower bud and Po=pollen).

After collection, the samples were immediately immersed in dry ice and the samples remained inside a styrofoam box with dry ice until delivery to the laboratory within 12 hours in all cases.

The pollen samples were processed before delivery for analysis. During transport from the field to lab, styrofoam boxes filled with gelox were used. The material was then stored in a refrigerator until pollen processing was carried out. The period between pollen collection, processing and delivery to the laboratory was always less than 8 days.

### 5.2. Laboratory Phase (Protein Quantification)

5.2.1. Preparation of Plant Material Samples used in the protein quantification experiment were macerated (frozen in liquid nitrogen) using TissueLyzer, with 30 oscillations per second, for 30 seconds. The process was repeated until a fine and homogeneous powder was obtained. Pollen samples were only lyophilized.

After maceration, the materials were directly lyophilized or stored in an Ultrafreezer (−70° C.) until lyophilization. All material was kept in liquid nitrogen during the processes. Lyophilization was performed using a Labconco lyophilizer, FreeZone model, at −56° C., for 4 days. Materials were lyophilized in 50 mL falcon tubes, with material up to a maximum of the 25 mL mark. The pollen was lyophilized in 15 mL falcon tubes.

After lyophilization, the samples were stored in Ultrafreezer −70° C. until the beginning of the quantification process.

### 5.2.2. Methodology Validation

The ELISA assays (young leaf, mature leaf, branch, flower bud and pollen) were validated in order to infer the accuracy, matrix effect, extraction efficiency, specificity, occurrence of false negatives, occurrence of false positives, linearity of dilution, precision and intermediate precision. During validation tests, Limits of Detection (LOD) and Limits of Quantification (LOQ) values were defined.

For the acceptance of protein quantification values, the assay must meet the criteria presented in Table 12.

TABLE 12
Acceptance criteria values for quantification by ELISA.

| Acceptance criteria | Value/Range |
|---|---|
| Theoretical % of retroactively | Highest concentration: 75-125% |
| calculated concentration of | Median concentrations: 80-120% |
| standards | Lowest concentration: 75-125% |
| CV (OD) of positive reference | ≤20% |
| standards | |
| R² of the standard curve | ≥0.98 |
| QC negative (Cry1Ab/2Aa) | <0.781 ng/mL (LOQ of the standard |
| | curve) |
| QC negative (Cry1Bb) | <3.13 ng/mL (LOQ of the standard |
| | curve) |
| QC negative (NPTII) | <1.0 ng/mL (LOQ of the standard |
| | curve) |

In addition, the concentration values measured in each of the tissues must be greater than the Limits of Detection (LOD) and (LOQ), established for each of the assays and matrices, as shown in Table 13.

TABLE 13
LOD and LOQ values for each evaluated test system.

| | LOD (ng/mL) | | | | LOQ (ng/mL/μ/g) | | | |
|---|---|---|---|---|---|---|---|---|
| Test System | Cry1Ab | Cry2Aa | Cry1Bb | NPTII | Cry1Ab | Cry2Aa | Cry1Bb | NPTII |
| Young Leaves | 0.390 | 0.390 | 3.130 | 0.500 | 0.6/0.24 | 0.6/0.24 | 6.2/1.24 | 1.0/0.4 |
| Mature Leaves | 0.390 | 0.390 | 3.130 | 0.500 | 0.6/0.96 | 0.6/0.24 | 6.2/1.24 | 1.0/0.4 |
| Stem | 0.390 | 0.390 | 3.130 | 0.500 | 0.6/0.60 | 0.6/0.25 | 6.2/1.24 | 1.0/0.4 |
| Root | 0.390 | 0.390 | 3.130 | — | 0.6/1.92 | 0.6/0.48 | 6.2/43.40 | — |
| Floral Bud | 0.390 | 0.390 | 3.130 | 0.500 | 0.6/0.60 | 0.6/0.96 | 6.2/2.48 | 1.0/0.4 |
| Pollen | 0.390 | 0.390 | 3.130 | 0.500 | 0.6/0.24 | 0.6/0.12 | 6.2/1.24 | 1.0/0.2 |

5.2.3. Protein quantification by ELISA (Cry1Ab)

Quantification analyzes of the expression of the Cry1Ab protein, in samples of eucalyptus tissues, were carried out.

A standard curve was mounted on all plates for Cry1Ab protein quantification (Table 14). The standard curve was obtained using the four-parameter logistic curve (4PL).

TABLE 14
Standard curve assembly for Cry1Ab proteins

| | | 1X | |
|---|---|---|---|
| | | PBST | Cry1Ab |
| Description | Standard Dilution | (μL) | (ng/mL) |
| Intermediate 1 | 10 μL of 1.0 mg/mL the stock | 1000* | 10000 |
| | solution | | |
| Intermediate 2 | 40 μL from the Intermediate 1 | 960 | 400 |
| Intermediate 3 | 200 μL from the Intermediate 2 | 1400 | 50.0 |
| Standard 1 | 800 μL from the Intermediate 3 | 800 | 25.0 |
| Standard 2 | 800 μL from the Standard 1 | 800 | 12.5 |
| Standard 3 | 800 μL from the Standard 2 | 800 | 6.25 |
| Standard 4 | 800 μL from the Standard 3 | 800 | 3.13 |
| Standard 5 | 800 μL from the Standard 4 | 800 | 1.56 |
| Standard 6 | 800 μL from the Standard 5 | 800 | 0.781 |
| Standard 7 | 800 μL from the Standard 6 | 800 | 0.390 |

Quantifications of the Cry1Ab protein in the samples were performed using the Agdia Kit (PSP 06200).

Plant material was weighed (0.03 g±0.001 g) and the extraction was performed using 3 mL of native protein extraction buffer (HEPES 150 mM; NaCL 2.5M; Triton x-100 10% (v/v); BSA 1% (v/v); PVP-10000 1.65% (m/v); Proclin-950 0.25% (v/v); Protease Inhibitor 10 μl/mL; pH 7.5).

In each well, 100 μl of the enzyme conjugate prepared was added, diluting 100 μl of the concentrated enzyme conjugate in 10 mL of RUB6 recipe (Agdia ACC 00470/0055). Then, 100 μL of the Exceptions extract was applied, after the necessary dilutions, according to the plate design. The plate was incubated for 2 hours at room temperature. The plate wells were then washed 5 times with 1×PBS-T. After washing, 100 μl of TMB substrate was added to each well. The plate was incubated for 10-15 minutes. After the incubation period, 100 μL of 1M hydrochloric acid was added to block the reaction. The reading was performed as described in item 9.3

The highest concentrations of Cry1Ab protein were observed in mature leaf tissues at 6 months after planting, averaging 35.69 μg of Cry1Ab proteins per gram of dry tissue. The lowest levels of Cry1Ab protein were found in root tissues at 24 months of age after planting, with about 1.76 μg of Cry1Ab protein per gram of tissue, as can be seen in Table 15. As a control sample (clone of conventional eucalyptus—FGN-K) were considered ND (not detectable) since the values found were lower than the Limit of Detection (<LOD) for Cry1Ab in all tissues considered.

TABLE 15
Results of Cry1Ab protein quantification in young leaves, mature leaves,
branches, floral bud roots and pollen collected at Farm 1 and Farm 2.

| | | | Cry1Ab | | | |
|---|---|---|---|---|---|---|
| | | | (μg/g DW) | | | |
| | | | | Standard | | |
| Matrix | Age | Material | Average* | Deviation | Min | Max |
| Young Leaf | 6 months | FGN-K | ND | — | ND | ND |
| | | Tg event no. 49 | 32.63 | 10.91 | 20.38 | 51.75 |
| | 12 months | FGN-K | ND | — | ND | ND |
| | | Tg event no. 49 | 29.39 | 9.48 | 18.61 | 40.21 |
| | 24 months | FGN-K | ND | — | ND | ND |
| | | Tg event no. 49 | 19.75 | 8.07 | 6.07 | 28.96 |
| Mature Leaf | 6 months | FGN-K | ND | — | ND | ND |
| | | Tg event no. 49 | 35.69 | 13.36 | 22.56 | 53.76 |
| | 12 months | FGN-K | ND | — | ND | ND |
| Stem | | Tg event no. 49 | 20.23 | 6.73 | 30.15 | 12.48 |
| | 24 months | FGN-K | ND | — | ND | ND |
| | | Tg event no. 49 | 14.73 | 3.06 | 10.40 | 18.70 |
| | 6 months | FGN-K | ND | — | ND | ND |
| | | Tg event no. 49 | 17.85 | 2.80 | 13.85 | 21.66 |
| | 12 months | FGN-K | ND | — | ND | ND |
| | | Tg event no. 49 | 9.63 | 3.48 | 5.68 | 13.72 |
| | 24 months | FGN-K | ND | — | ND | ND |
| | | Tg event no. 49 | 6.63 | 1.3 | 4.84 | 8.01 |
| Root | 6 months | FGN-K | ND | — | ND | ND |
| | | Tg event no. 49 | 5.35 | 1.87 | 2.88 | 7.61 |
| | 12 months | FGN-K | ND | — | ND | ND |
| | | Tg event no. 49 | 3.73 | 0.73 | 2.81 | 4.59 |
| | 24 months | FGN-K | ND | — | ND | ND |
| | | Tg event no. 49 | 1.76 | 0.40 | 1.47 | 2.04 |
| Floral Bud | Flowering | FGN-K | ND | — | ND | ND |
| | | Tg event no. 49 | 7.71 | 0.18 | 7.57 | 7.92 |
| Pollen | Flowering | FGN-K | ND | — | ND | ND |
| | | Tg event no. 49 | 1.79 | 0.29 | 1.46 | 2.00 |

ND (Not detectable) =< LOD, that is, the protein level found was lower than the Limit of Detection of the analytical method.
*Average between two locations (Farm 1 and Farm 2).

### 5.2.4. Protein Quantification by ELISA (Cry2Aa)

The quantification analyzes of the expression of the Cry2Aa protein, in eucalyptus tissue samples, were carried out.

A standard curve was mounted on all plates for Cry2Aa protein quantification (Table 16). The standard curve was obtained using the four-parameter logistic curve (4PL).

TABLE 16
Standard curve assembly for Cry2Aa proteins

| | | 1X | |
|---|---|---|---|
| | | PBST | Cry2Aa |
| Description | Standard Dilution | (μL) | (ng/mL) |
| Intermediate 1 | 10 μL of 1.0 mg/mL the stock | 1000* | 10000 |
| | solution | | |
| Intermediate 2 | 40 μL from the Intermediate 1 | 960 | 400 |
| Intermediate 3 | 200 μL from the Intermediate 2 | 1400 | 50.0 |
| Standard 1 | 800 μL from the Intermediate 3 | 800 | 25.0 |
| Standard 2 | 800 μL from the Standard 1 | 800 | 12.5 |
| Standard 3 | 800 μL from the Standard 2 | 800 | 6.25 |
| Standard 4 | 800 μL from the Standard 3 | 800 | 3.13 |
| Standard 5 | 800 μL from the Standard 4 | 800 | 1.56 |
| Standard 6 | 800 μL from the Standard 5 | 800 | 0.781 |
| Standard 7 | 800 μL from the Standard 6 | 800 | 0.390 |

Quantifications of the Cry2Aa protein in the samples were performed using the Agdia Kit (PSP 05801), following the procedures described in POP.BM.030 and RG028.2022.

Plant material was weighed (0.03 g±0.001 g) and the extraction was performed using 3 mL of native protein extraction buffer (HEPES 150 mM; NaCL 2.5M; Triton x-100 10% (v/v); BSA 1% (v/v); PVP-10000 1.65% (m/v); Proclin-950 0.25% (v/v); Protease Inhibitor 10 μl/ml; pH 7.5).

In each well, 100 μL of the enzyme conjugate prepared was added by diluting 100 μL of the concentrated enzyme conjugate in 10 mL of RUB6 buffer (Agdia ACC 00470/0055). Then, 100 μL of sample extract were applied, after the necessary dilutions, according to the plate design. The plate was incubated for 1 hour at room temperature. The plate wells were then washed 5 times with 1×PBS-T. After washing. 100 μl of TMB substrate was added to each well. The plate was incubated for 10-15 minutes. After the incubation period, 100 μL of 1M hydrochloric acid was added to block the reaction. The reading was performed as described in item 9.3.

The mean expression values for Cry2Aa in the Tg event no. 49 eucalyptus samples collected at Farm 1 and Farm 2 ranged from 0.23 μg/g in branches at 24 months of age after planting to 9.09 μg/g of dry tissue weight in young leaves at 6 months after planting (Table 17). It is noteworthy that the Cry2Aa protein was not detected in root tissues at 12 and 24 months after planting.

In none of the tissues analyzed was the presence of Cry2Aa protein detected in control samples (conventional eucalyptus clone—FGN-K). All control samples were considered ND (not detectable) since the OD values found were less than the Limit of Detection (<LOD).

TABLE 17
Results of Cry2Aa protein quantification in young leaves, mature leaves,
branches, floral bud roots and pollen collected at Farm 1 and Farm 2.

| | | | Cry1Ab | | | |
|---|---|---|---|---|---|---|
| | | | (μg/g DW) | | | |
| | | | | Standard | | |
| Matrix | Age | Material | Average* | Deviation | Min | Max |
| Young Leaf | 6 | FGN-K | ND | — | ND | ND |
| | months | Tg event no. 49 | 9.09 | 0.77 | 8.12 | 9.73 |
| | 12 | FGN-K | ND | — | ND | ND |
| | months | Tg event no. 49 | 5.44 | 2.13 | 3.10 | 7.27 |
| | 24 | FGN-K | ND | — | ND | ND |
| | months | Tg event no. 49 | 5.53 | 3.25 | 2.62 | 9.38 |
| Mature Leaf | 6 | FGN-K | ND | — | ND | ND |
| | months | Tg event no. 49 | 8.65 | 0.5 | 7.89 | 9.24 |
| | 12 | FGN-K | ND | — | ND | ND |
| | months | Tg event no. 49 | 5.25 | 0.94 | 3.95 | 6.44 |
| | 24 | FGN-K | ND | — | ND | ND |
| | months | Tg event no. 49 | 4.36 | 1.99 | 1.36 | 6.65 |
| Stem | 6 | FGN-K | ND | — | ND | ND |
| | months | Tg event no. 49 | 0.71 | 0.24 | 0.35 | 1.05 |
| | 12 | FGN-K | ND | — | ND | ND |
| | months | Tg event no. 49 | 0.4 | 0.10 | 0.37 | 0.51 |
| | 24 | FGN-K | ND | — | ND | ND |
| | months | Tg event no. 49 | 0.23 | 0.07 | 0.20 | 0.33 |
| Root | 6 | FGN-K | ND | — | ND | ND |
| | months | Tg event no. 49 | 0.72 | 0.09 | 0.61 | 0.77 |
| | 12 | FGN-K | ND | — | ND | ND |
| | months | Tg event no. 49 | ND | — | ND | ND |
| | 24 | FGN-K | ND | — | ND | ND |
| | months | Tg event no. 49 | ND | — | ND | ND |
| Floral Bud | Flowering | FGN-K | ND | — | ND | ND |
| | | Tg event no. 49 | 3.84 | 0.91 | 2.94 | 4.75 |
| Pollen | Flowering | FGN-K | ND | — | ND | ND |
| | | Tg event no. 49 | 0.24 | 0.02 | 0.20 | 0.24 |

ND (Not detectable) =< LOD, that is, the protein level found was lower than the Limit of Detection of the analytical method.
*Average between two locations (Farm 1 and Farm 2).

### 5.2.5. Protein Quantification by ELISA (Cry1Bb)

Quantification analyzes of Cry1Bb protein expression in eucalyptus tissue samples were performed.

A standard curve was mounted on all plates for Cry1Bb protein quantification (Table 18). The standard curve was obtained using the four-parameter logistic curve (4PL).

TABLE 18
Standard curve assembly for Cry1Bb proteins

| | | 1X | |
|---|---|---|---|
| | | PBST | Cry2Aa |
| Description | Standard Dilution | (μL) | (ng/mL) |
| Intermediate 1 | 10 μL of 1.0 mg/mL the stock | 1000* | 10000 |
| | solution | | |
| Intermediate 2 | 40 μL from the Intermediate 1 | 960 | 400 |
| Standard 1 | 200 μL from the Intermediate 2 | 800 | 200 |
| Standard 2 | 500 μL from the Standard 1 | 500 | 100 |
| Standard 3 | 500 μL from the Standard 2 | 500 | 50 |
| Standard 4 | 500 μL from the Standard 3 | 500 | 25 |
| Standard 5 | 500 μL from the Standard 4 | 500 | 12.5 |
| Standard 6 | 500 μL from the Standard 5 | 500 | 6.25 |
| Standard 7 | 500 μL from the Standard 5 | 500 | 3.13 |

Quantifications of the Cry1Bb protein in the samples were performed using the Abraxis Kit (PN 599100), developed on demand, following the procedures designated by the manufacturer.

Plant material was weighed (0.03 g±0.001 g) and the extraction was performed using 3 mL of 1×-Tris Borate (Trisma base 100 mM; Na2B4O7×10H2O 100 mM; MgCl2×6H2O 5 mM; Tween-20 0.05% (v/v), pH to 7.8).

Initially, 100 μL of sample extract were applied, after necessary dilutions, according to the plate design. The plate was incubated for 30 minutes at room temperature. The plate wells were then washed 5 times with 1×PBS-T. After washing. 100 μL of enzyme conjugate, supplied ready-to-use by the manufacturer, were added. The plate was then incubated for 30 minutes at room temperature. The plate wells were then washed 5 times with 1×PBS-T. After washing. 100 μl of TMB substrate was added to each well. The plate was incubated for 10-15 minutes. After the incubation period, 100 μL of 1M hydrochloric acid was added to block the reaction. The reading was performed as described in item 9.3.

As with the measurements of Cry2Aa protein, the highest concentrations of Cry1Bb protein were observed in tissues of young leaves at 6 months after planting, with an average of 5.58 ptg of Cry1Bb protein per gram of dry tissue. The lowest values were also observed in tissues of young leaves, but at 24 months after planting, with an average of 2.11 μg/g of dry weight (Table 19).

It is worth mentioning that the concentrations of the Cry1Bb protein could only be measured in the tissues of young leaves and mature leaves, with the expression in these tissues being lower than the limit of detection (LOD). Such results were expected, since the expression of the Cry1Bb protein is controlled by the promoter of the Rubisco protein, which favors the expression only in photosynthetic tissues.

Control samples (conventional eucalyptus clone—FGN-K) were considered ND (not detectable) since the values found were lower than the Limit of Detection (<LOD) for Cry1Bb in all evaluated tissues.

Results of Cry1Bb protein quantification in young leaves, mature leaves, branches, floral bud roots and pollen collected at Farm 1 and Farm 2.

TABLE 19
Results of Cry1Bb protein quantification in young leaves, mature leaves,
branches, floral bud roots and pollen collected at Farm 1 and Farm 2.

| | | | Cry1Bb | | | |
|---|---|---|---|---|---|---|
| | | | (μg/g DW) | | | |
| | | | | Standard | | |
| Matrix | Age | Material | Average* | Deviation | Min | Max |
| Young Leaf | 6 months | FGN-K | ND | — | ND | ND |
| | | Tg event no. 49 | 4.49 | 0.91 | 3.36 | 5.8 |
| | 12 months | FGN-K | ND | — | ND | ND |
| | | Tg event no. 49 | 3.82 | 0.58 | 3.02 | 4.71 |
| | 24 months | FGN-K | ND | — | ND | ND |
| | | Tg event no. 49 | 2.11 | 0.93 | 0.71 | 3.22 |
| Mature Leaf | 6 months | FGN-K | ND | — | ND | ND |
| | | Tg event no. 49 | 5.89 | 1.81 | 4.45 | 8.33 |
| | 12 months | FGN-K | ND | — | ND | ND |
| | | Tg event no. 49 | 2.37 | 0.25 | 2.37 | 3.07 |
| | 24 months | FGN-K | ND | — | ND | ND |
| | | Tg event no. 49 | 2.72 | 0.54 | 2.03 | 3.26 |
| Stem | 6 months | FGN-K | ND | — | ND | ND |
| | | Tg event no. 49 | ND | — | ND | ND |
| | 12 months | FGN-K | ND | — | ND | ND |
| | | Tg event no. 49 | ND | — | ND | ND |
| | 24 months | FGN-K | ND | — | ND | ND |
| | | Tg event no. 49 | ND | — | ND | ND |
| Root | 6 months | FGN-K | ND | — | ND | ND |
| | | Tg event no. 49 | ND | — | ND | ND |
| | 12 months | FGN-K | ND | — | ND | ND |
| | | Tg event no. 49 | ND | — | ND | ND |
| | 24 months | FGN-K | ND | — | ND | ND |
| | | Tg event no. 49 | ND | — | ND | ND |
| Floral Bud | Flowering | FGN-K | ND | — | ND | ND |
| | | Tg event no. 49 | ND | — | ND | ND |
| Pollen | Flowering | FGN-K | ND | — | ND | ND |
| | | Tg event no. 49 | ND | — | ND | ND |

ND (Not detectable) =< LOD, that is, the protein level found was lower than the Limit of Detection of the analytical method.
*Average between two locations (Farm 1 and Farm 2).

### 5.2.6. Protein Quantification by ELISA (NPTII)

Quantification analyzes of NPTII protein expression in eucalyptus tissue samples were performed.

A standard curve was mounted on all plates for NPTII protein quantification.

(Table 20). The standard curve was obtained using the four-parameter logistic curve (4PL).

TABLE 20
Standard curve for NPTII using commercial
standard protein supplied by Agdia

| | | 1X | |
|---|---|---|---|
| | | PBST | NPTII |
| Description | Standard Dilution | (μL) | (ng/mL) |
| Intermediate 1 | 10 μL of 0.180 mg/mL Stock | 1790 | 1000 |
| | Solution | | |
| Intermediate 2 | 80 μL from the Intermediate 1 | 920 | 80 |
| Standard 1 | 300 μL from the Intermediate 2 | 1200 | 16.0 |
| Standard 2 | 750 μL from the Standard 1 | 750 | 8.00 |
| Standard 3 | 750 μL from the Standard 2 | 750 | 4.00 |
| Standard 4 | 750 μL from the Standard 3 | 750 | 2.00 |
| Standard 5 | 750 μL from the Standard 4 | 750 | 1.00 |
| Standard 6 | 750 μL from the Standard 5 | 750 | 0.500 |

Quantifications of the NPTII protein in the samples were performed using the Agdia Kit (PSP 73000).

Plant material was weighed (0.03 g±0.001 g) and the extraction was performed using 3 mL of PEB buffer (provided in the Kit). 100 μL of sample extract were applied, after necessary dilutions, according to the plate design. The plate was incubated for 2 hours at room temperature. The plate wells were then washed 5 times with PBS-T. Subsequently, 100 μL of the enzyme conjugate (100 μL of antibody A (Bottle A)+100 μL of antibody B (Bottle B) were added for each 10 mL of the diluted MRS2 solution). The plate was incubated for 2 hours at room temperature, and then washed 5 times with 1×PBS-T solution. After washing, 100 μl of TMB substrate was added to each well. The plate was incubated for 15 minutes. After the incubation period, 100 μL of 1M hydrochloric acid was added to block the reaction. The reading was performed as described in item 9.3.

The mean expression values for NPTII in the Tg event no. 49 eucalyptus samples collected at Farm 1 and Farm 2 ranged from 0.22 μg/g in young leaves at 24 months of age after planting to 0.43 μg/g of dry weight in flower buds (Table 21). In pollen samples collected in a greenhouse, NPTII values were lower than the limit of detection of the method (<LOD).

Control samples (conventional eucalyptus clone—FGN-K) were considered ND (not detectable) since the values found were lower than the Limit of Detection (<LOD) for NPTII in all evaluated tissues.

TABLE 21
Results of NPTII protein quantification in young leaves, mature
leaves, branches, flower buds and pollen collected at Farm 1.

| | | | Cry1Bb | | | |
|---|---|---|---|---|---|---|
| | | | (μg/g DW) | | | |
| | | | | Standard | | |
| Matrix | Age | Material | Average* | Deviation | Min | Max |
| Young Leaf | 6 months | FGN-K | ND | — | ND | ND |
| | | | 0.41 | 0.11 | 0.29 | 0.51 |
| | 12 months | FGN-K | ND | — | ND | ND |
| | | Tg event no. 49 | 0.24 | 0.01 | 0.22 | 0.25 |
| | 24 months | FGN-K | ND | — | ND | ND |
| | | Tg event no. 49 | 0.22 | 0.02 | 0.20 | 0.25 |
| Mature Leaf | 6 months | FGN-K | ND | — | ND | ND |
| | | Tg event no. 49 | 0.34 | 0.10 | 0.24 | 0.52 |
| | 12 months | FGN-K | ND | — | ND | ND |
| | | Tg event no. 49 | 0.32 | 0.11 | 0.25 | 0.45 |
| | 24 months | FGN-K | ND | — | ND | ND |
| | | Tg event no. 49 | 0.28 | 0.09 | 0.21 | 0.40 |
| Stem | 6 months | FGN-K | ND | — | ND | ND |
| | | Tg event no. 49 | 0.30 | 0.02 | 0.27 | 0.32 |
| | 12 months | FGN-K | ND | — | ND | ND |
| | | Tg event no. 49 | 0.34 | 0.08 | 0.25 | 0.40 |
| | 24 months | FGN-K | ND | — | ND | ND |
| | | Tg event no. 49 | 0.30 | 0.04 | 0.25 | 0.33 |
| Root | 6 months | FGN-K | ND | — | ND | ND |
| | | Tg event no. 49 | 0.43 | 0.06 | 0.36 | 0.51 |
| | 12 months | FGN-K | ND | — | ND | ND |
| | | Tg event no. 49 | ND | — | ND | ND |
| | 24 months | FGN-K | ND | — | ND | ND |
| | | | 0.41 | 0.11 | 0.29 | 0.51 |
| Floral Bud | Flowering | FGN-K | ND | — | ND | ND |
| | | Tg event no. 49 | 0.24 | 0.01 | 0.22 | 0.25 |
| Pollen | Flowering | FGN-K | ND | — | ND | ND |
| | | Tg event no. 49 | 0.22 | 0.02 | 0.20 | 0.25 |

ND (Not detectable) =< LOD, that is, the protein level found was lower than the Limit of Detection of the analytical method.
*Average between two locations (Farm 1 and Farm 2).

### 5.3. Data Analysis

Data were analyzed using SkanIt RE Version 5.0 software. For concentration calculations interpolating the values of the standard curve, each of the wells was read at wavelengths of 620 nm and 450 nm, then the values of the respective blanks were subtracted and then subtracted the OD620 values from the OD450 values.

The calculation of the actual protein expression level in dry weight (ng of protein per mg of tissue) was performed according to the equation below. Next, the values in μg/g of tissue dry weight were presented.

$$\frac{C \times V \times D}{W \times 1000 \frac{mg}{g}} = protein concentration in \frac{μ g}{g} of dry tissue$$

Where:
C=Analyte concentration in the sample (ng/mL)
V=Extraction Volume (mL)
D=Dilution
W=Sample weight (g)

### 5.4. Reagents and Solutions

Cry1Ab-1Ac Test Kit, Agdia, PSP06200/0480
Cry2Aa Test Kit, Agdia, PSP05801/0480
Cry1Bb Test Kit, Eurofins-Abraxis, PN599100
Phosphate Buffered Saline with TWEEN® 20 (PBST), pH 7.4, Agdia, Part No. ACC 00501
Hydrochloric acid, concentrated HCl, ACS grade
Ultrapure water
Trizma Base, Sigma, Part No. T6066
Tween20, Sigma, Part No. P7949
Magnesium Chloride Hexahydrate, ACS Grade, Sigma, Part No. M2670
Sodium tetraborate decahydrate, Sigma, Part No S9640
Hepes, Sigma, Part No. H3375
NaCl, Sigma, Part No. S3014
Triton x-100, Sigma, Part No T8787
Protease inhibitor cocktail, Sigma, Part P9599
PVPP, Sigma, Part No. 77627

### Example 14: Characterization of Cry Protein Synergy (Cry1Ab, Cry2Aa and Cry1Bb) in the Control of Thyrinteina arnobia

### 1. Objective

Evaluate the efficacy of Cry proteins in the control of caterpillars of the species Thyrinteina arnobia, using isolated and combined proteins.

### 2. Test System

The test system of the present study was composed of caterpillars of the species Thyrinteina arnobia.

Justification for the choice of the species: Reference species as Eucalyptus pest (Bragança et al., 1998; Oliveira et al., 2011; Barbosa et al., 2016).

Origin of organisms: Caterpillars originally collected from Tres Lagoas/MS.

Age: Caterpillars of 1st instar (age less than 3.8 days) (Wilcken C. F., 1996).

Generation: 5th generation laboratory.

### 3. Test Item

The test items were composed of Cry proteins applied to the artificial diet surface according to treatments described in Table 22.

TABLE 22
Test item used for conducting the experiment

| Treatment | Protein Concentration in μg/g | | | | | | | | | | | | |
|---|---|---|---|---|---|---|---|---|---|---|---|---|---|
| Cry1Ab | 0.007 | 0.015 | 0.029 | 0.059 | 0.117 | 0.234 | 0.469 | 0.938 | 1.875 | 3.75 | 7.5 | 15 | 30 |
| Cry2Aa | 0.007 | 0.015 | 0.029 | 0.059 | 0.117 | 0.234 | 0.469 | 0.938 | 1.875 | 3.75 | 7.5 | 15 | 30 |
| Cry1Bb | 0.007 | 0.015 | 0.029 | 0.059 | 0.117 | 0.234 | 0.469 | 0.938 | 1.875 | 3.75 | 7.5 | 15 | 30 |
| Cry1Ab + Cry2Aa | 0.007 | 0.015 | 0.029 | 0.059 | 0.117 | 0.234 | 0.469 | 0.938 | 1.875 | 3.75 | 7.5 | 15 | 30 |
| Cry1Ab + Cry1Bb | 0.007 | 0.015 | 0.029 | 0.059 | 0.117 | 0.234 | 0.469 | 0.938 | 1.875 | 3.75 | 7.5 | 15 | 30 |
| Cry2Aa + Cry1Bb | 0.007 | 0.015 | 0.029 | 0.059 | 0.117 | 0.234 | 0.469 | 0.938 | 1.875 | 3.75 | 7.5 | 15 | 30 |
| Cry1Ab + Cry2Aa + | 0.007 | 0.015 | 0.029 | 0.059 | 0.117 | 0.234 | 0.469 | 0.938 | 1.875 | 3.75 | 7.5 | 15 | 30 |
| Cry1Bb | | | | | | | | | | | | | |
| Buffer | | | | | | | | | | | | | |

### 4. Reference Item

Reference Item 1 (positive control) was the Dipel product applied to the diet surface. The preparation of Dipel followed the commercial dosage where 1 mL of the product was diluted in 1000 mL of water. This treatment is expected to result in 100% mortality by the seventh day of evaluation.

Reference Item 2 (negative control) was water applied to the diet surface. This reference treatment item is expected to result in up to 50% of caterpillar mortality on the seventh day of evaluation.

Reference Item 3 (negative control) was the addition of the buffer CAPS 10 mM (pH 10) (used in the dilution of Cry proteins) on the surface of the artificial diet. This reference treatment item is expected to result in up to 50% of caterpillar mortality on the seventh day of evaluation.

TABLE 23
Reference treatment items used.

Dipel (1:1000)
Water
Buffer

### 5. Study Chronology

TABLE 24

| Stages | Stage start date | Stage finish date |
|---|---|---|
| 1. Birth of caterpillars for | October-November | October-November |
| the test; | | |
| 2. Processing of samples; | October-November | October-November |
| 3. Exposure to caterpillars' | October-November | October-November |
| consumption; | | |
| 4. Count of dead caterpillars; | October-November | October-November |
| 5. Finalization of the Raw | October-November | October-November |
| Data Notebook; | | |
| 6. Final Report | December | December |

### 6. Evaluation of Results

### 6.1. Description of Materials and Methods

### 6.1.1. Proteins Description and Preparation

Proteins used in this study (Cry1Ab, Cry1Bb and Cry2Aa) were produced on demand by the company Fraunhofer-Gesellschaft, using Pseudomonas fluorescens as host organism.

Lyophilized proteins were resuspended in buffer CAPS 10 mM (pH 10) to a stock solution of 1 mg/mL and stored at −80° C. until the preparation of the treatments.

Treatments were prepared making a serial dilution starting in the higher treatment concentration (30 μg/mL) and then diluting it by 2.

The nominal protein concentration of a treatment refers to the fixed weight of protein in the combination of all proteins presented in any of the planned experimental solutions. For example, a treatment point of 30 μg/mL will contain 30 μg/mL of protein A if the treatment is composed by a single protein (ex: Cry1Ab), 15 μg/mL of protein A+15 μg/mL of protein B if the treatment is composed by a pair of proteins (ex: Cry1 Ab+Cry2Aa), or 10 μg/mL of protein A+10 μg/mL of protein B+10 μg/mL of protein C if the treatment is composed by a triple of proteins (ex: Cry1Ab+Cry2Aa+Cry1Bb).

For Technical Specifications of each protein used see Section 4 Example 13 above

### 6.1.2. Experiment Setup

The caterpillars used after egg hatching remained isolated and stored in an air-conditioned room at 17±2° C. Cry1Bb, Cry2Aa and Cry1Ab proteins were used in the synergy assay to measure the toxicity in 1° instar caterpillars of Thyrinteina arnobia. The artificial diet was prepared as described above. The artificial diet was added to 2 mL microtubes, in each microtube 0.5 mL of diet was applied and ready for use after 24.

The protein concentrations in the bioassay study were defined based on preliminary LD50 experiments 10 μL of the different protein concentrations (see Table 22) was added to the artificial diet surface of each of the B/proteins isolated and combined as described (Cry1Bb, Cry2Aa, Cry1Ab, Cry1Bb+Cry2Aa, Cry1Bb+Cry1Ab, Cry1Ab+Cry2Aa and Cry2Aa+Cry1Ab+Cry1Bb).

Protein diets were maintained at room temperature for protein solution absorption in the artificial diet, this process takes 24 hours. Subsequently, a caterpillar of 1st instar was added to each microtube, which had its lids previously drilled and later with the aid of voile fabric closed to allow gas exchange, 15 replicates were used for each treatment. Likewise, the reference items Dipel, water and buffer (the same buffer used in the dilution of proteins), were also applied to the surface of the artificial diet. After placing the caterpillars, the microtubes were kept in air-conditioned rooms at 25° C. (+4° C.) with photoperiod control of 12/12.

All microtubes were individually identified, containing information of the start date of the experiment, responsible and treatment. Caterpillar mortality was evaluated daily for 7 days. The results were analyzed by Tukey's method, using script written in R 4.2.1 (Agricolae 1.3.5) and Probit for determination of LD50.

### 6.2. Evaluation and Discussion of Results

Positive reference item 1 consisting of Dipel application presented 100% mortality as expected and presupposition for validation of the experiment. Regarding the negative reference items, with water application (2) and buffer solution (3), both presented mortality below 50%. All reference items presented results within the expected range, thus validating the methodology used.

The mortality rate data for each Cry protein used individually or combined with one or two additional proteins were submitted to “Probit” analysis to determine the LD50 generating the equations described in Table 25.

TABLE 25
Formulas generated after Probit evaluation
to determine the LD50 of each treatment

| Protein | Function |
|---|---|
| Cry1Bb | y = 1.6156x + 5.4505 |
| Cry2Aa | y = 1.7927x + 5.7209 |
| Cry1Ab | y = 2.0156x + 5.2463 |
| Cry1Ab + Cry2Aa | y = 1.5673x + 5.8160 |
| Cry1Ab + Cry1Bb | y = 1.1963x + 5.7341 |
| Cry2Aa + Cry1Bb | y = 1.6022x + 5.7723 |
| Cry1Ab + Cry2Aa + Cry1Bb | y = 1.8964x + 6.3712 |

X = Toxin dose logarithm (μg/mL)
Y = “Probit” value to be converted into % mortality (Use of the Probit table)

The calculation of LD50 was performed by replacing the variable Y by 5, corresponding number in the “Probit” table for 50% of mortality, finding the LD50 for each treatment (Table 26)

TABLE 26
LD50 calculated for each treatment using Probit calculation

| Proteins | LD50 (μg/mL) |
|---|---|
| Cry1Ab | 0.75 |
| Cry2Aa | 0.39 |
| Cry1Bb | 0.52 |
| Cry1Ab + Cry2Aa | 0.3 |
| Cry1Ab + Cry1Bb | 0.24 |
| Cry2Aa + Cry1Bb | 0.33 |
| Cry1Ab + Cry2Aa + Cry1Bb | 0.18 |

Example: Cry1Bb 5 = 1.6156x + 5.4505 x = −0.278843
LD50 = X Antilog 10\hat{ }^{(-0.278843)} LD50 = 0.52 μg/mL

The Cry1Ab protein, when tested alone, presented the highest LD50 among all treatments with a value of 0.75 μg/mL, followed by the Cry1Bb protein that presented individual LD50 of 0.52 μg/m. The Cry2Aa protein presented the lowest individual LD50 with a value of 0.39 μg/mL, but a value even higher than the higher LD50 presented for the combined use of two or more proteins.

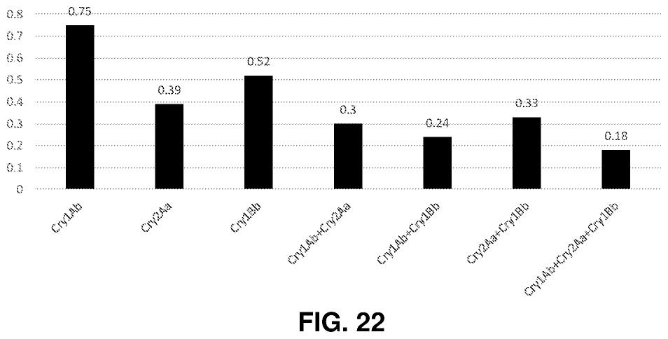

When used in pairs, any association between the tested proteins significantly reduced the LD50 compared to its individual use (FIG. 22). The lowest LD50 was the combined use of Cry1Ab+Cry1Bb proteins with LD50 of 0.24 μg/mL, these proteins when used individually presented the two largest LD50, thus demonstrating positive synergy.

When using the three proteins together Cry1Ab+Cry2Aa+Cry1Bb, the lowest LD50 was found among all treatments (LD50 0.18 μg/mL), demonstrating a synergistic effect indicating that this combination of proteins has the potential to conform a pyramided plant.

The simultaneous use of proteins does not always positively result in the control of caterpillars, the intrinsic relationship between toxins and their receptors can lead to antagonistic or synergistic effects (Lemes, 2012). Cry1Aa and Cry1Ab proteins for Lymantria disparate (Lepidoptera: Lymantriidae) (Linnaeus, 1758) showed antagonistic results, reducing mortality on the tested insect, but Cry1Aa has a synergistic effect between Cry1 Aa and Cry1 Ac for the same species (LEE et al, 1996).

It is important to highlight that at the onset of feeding with artificial diet containing Cry proteins the caterpillar consumes only a small initial part, this is because Cry protein poisoning causes an immediate food inhibition (Van Frankenhuyzen, 1993), thereby reducing food consumption and consequently exposure to Cry proteins present (Berlitz & Fiuza, 2004).

When this small initial amount is consumed by the caterpillar contains only one protein, it acts only on one site of action, however when that same small initial amount contains two or more Cry proteins the action occurs at several sites, significantly compromising the survival of the caterpillar, as observed.

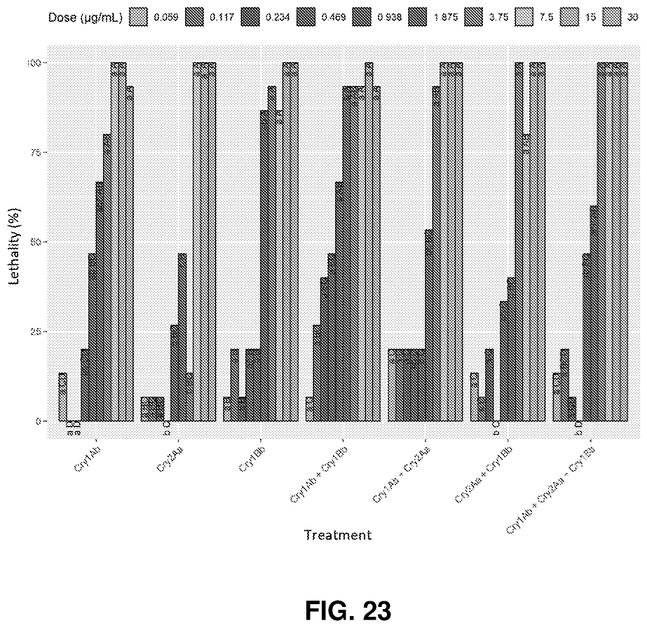

The combinations of two or more proteins when in high dose do not present significant increase on the effect of caterpillar mortality when compared to their individual use (FIG. 23), rendering the observed synergy insignificant, since at these concentrations only the presence of one of the proteins is sufficient to cause mortality above LD50.

It is necessary to highlight the impairment of the caterpillars' development submitted to protein consumption at a concentration below LD50 (sublethal), although alive on the seventh day after the beginning of exposure, the development of these caterpillars was totally compromised as evidenced in FIG. 23. Effect similar to Cry1Aa proteins, Cry1Ac, Cry1Ca, Vip3A(1), Vip3A(2) and Vip3A(3) when offered to H. virescens caterpillars at concentrations lower than concentrations that cause 50% mortality, reducing larval weight and causing effects which are classified as sublethal, underdeveloped caterpillars cause lower crop damage, biological control agents are more vulnerable, and when at the end of the cycle, the involvement during the initial phase entails not only the formation of the pupa, but also the emergence of the adult as well as its fecundity (Gore et al., 2005. Rudders 2004).

Mascarenhas & Luttrell (1997) also observed a reduction in the weight of H. zea caterpillars fed Bt cotton, expressing Cry1A from B. thuringiensis compared to insects fed a conventional cotton cultivar. Similar results were found by Eizaguirre et al. (2005), when evaluating the sublethal effect of B. thuringiensis on the larval development of Sesamia nonagrioides (Lepidoptera: Noctuidae) (Lefèvbre) and By Polanczyk & Alves (2005) when verifying the sublethal effect of some isolates of B. thuringiensis on S caterpillars.

It can be concluded that any association between Cry1Ab, Cry2Aa and Cry1Bb either as a doublet or a triplet, provides an advantage over its individual use for control of Thyrinteina arnobia. Cry1Ab, Cry2Aa and Cry1Bb are excellent candidates for pyramided use in plants for the control of T. arnobia. Dose lower than LD50 of Cry1Ab, Cry2Aa and Cry1Bb proteins used individually or simultaneously cause sublethal effects on T. arnobia caterpillars.

### Example 15: Binding Competition Assays with Thyrinteina arnobia BBMV

### Goal:

Propose a binding model for the following pesticidal proteins (PP): Cry1Bb, Cry1Ab and Cry2Aa in the midgut of T. arnobia and determine if these PP share binding sites or have unique receptors that suggest unique MOA for each PP. A unique binding site for each PP and potentially 3 MOA is important for the durability of an insect-resistant eucalyptus. It will help apply smart integrated resistance management to prevent the target pest from developing resistance.

### Method

Thyrinteina arnobia midgut brush border membrane vesicles (BBMVs) were prepared as described in Gouffon, C. V., A. Van Vliet, J. Van Rie, S. Jansens, and J. L. Jurat-Fuentes. “Binding sites for Bacillus thuringiensis Cry2Ae toxin on heliothine brush border membrane vesicles are not shared with Cry1A, Cry1F, or Vip3A toxin.” Applied and environmental microbiology 77, no. 10 (2011): 3182-3188.

Homologous and heterologous competition binding assays were performed with 20 μg/μl of T. arnobia BBMV. This BBMV concentration was selected from the results of preliminary assays as being below saturation. Binding reactions included 0.1 nM of 125I-PP and/or biotin-labeled PPs alone or in the presence of increasing excess (5, 10, 30, 50, 100, 300, 500 and 1000-fold) of unlabeled Cry1Ab, Cry2Aa and Cry1Bb as competitors. Reactions were incubated at room temperature in binding buffer (PBS 0.1% BSA) for 1 hour, and then bound toxin and BBMV were recovered in pellets after centrifugation at 14,500 rpm for 10 min. Pellets were washed with ice-cold binding buffer (0.5 ml) and centrifuged as before. The amount of labeled 125I-PPs remaining bound in the final BBMV pellet was quantified in a gamma counter (Wizard2, Perkin Elmer). The amount of labeled biotin-labeled PPs remaining bound in the final BBMV pellet was quantified by western blot.

Binding in the absence of a competitor was considered as 100% to estimate the percentage of labeled PPs remaining bound in the presence of competitors. Each data point is the mean and corresponding standard error from two independent experiments performed in duplicate.

### Results

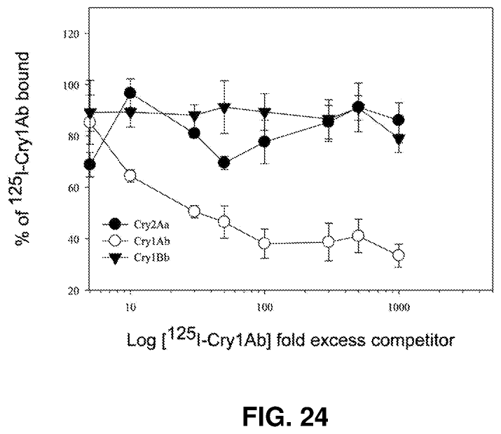

FIG. 24 shows that 125I-Cry1Ab competed with unlabeled Cry1Ab, Cry1Bb and Cry2Aa do not compete with 125I-Cry1Ab in binding to T. arnobia BBMV.

### Conclusion

No Cry1Ab binding sites are shared with Cry1Bb or Cry2Aa

### Results

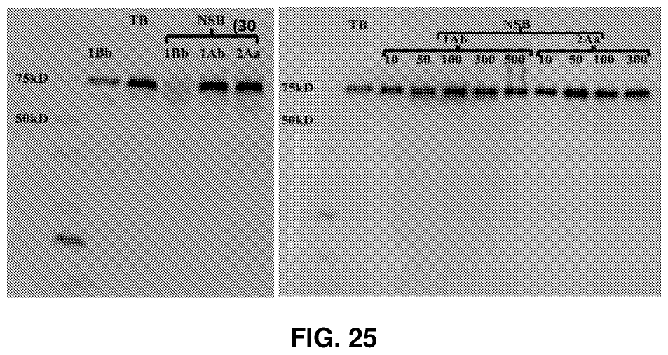

FIG. 25 shows Biotin-labeled Cry1Bb competed only with unlabeled Cry1Bb, Cry1Ab and Cry2Aa do not compete with biotin-labeled Cry1Bb in binding to T. arnobia BBMV.

### Conclusion

No Cry1Bb binding sites shared with Cry1Ab or Cry2Aa

### Results

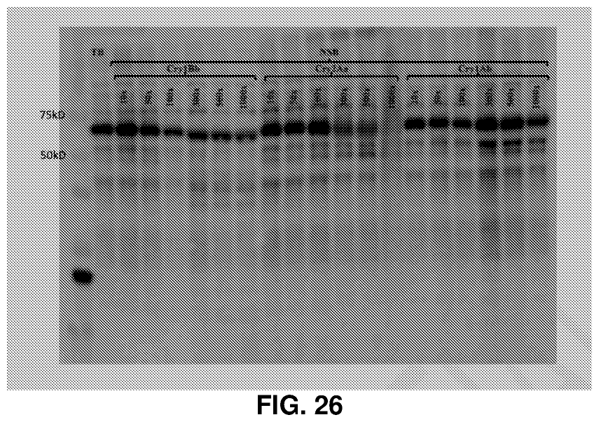

FIG. 26 shows that Biotin-labeled Cry2Aa competed only with unlabeled Cry2Aa mainly with 300-1000-fold. Cry1Ab and Cry1Bb do not compete with biotin-labeled Cry2Aa in binding to T. arnobia BBMV.

### Conclusion

No Cry2Aa binding sites shared with Cry1Ab or Cry1Bb

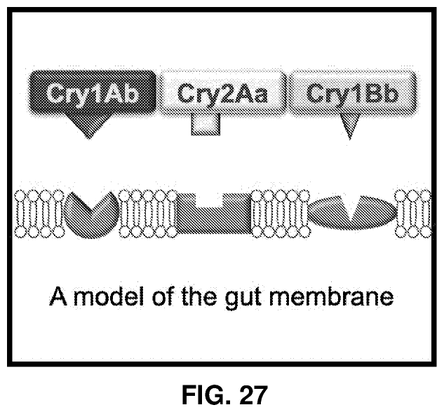

As illustrated in FIG. 27 these results indicate that each pesticidal protein has a unique binding site/receptor that reflects a specific mode of action. There is no binding competition between these PPs, and each PP acts independently. This significantly reduces the chance of resistant development to the insect resistance eucalyptus baring the Triple Bt PP since it is unlikely that the target pest will evolve in such away that affects all 3 MOA in a short time.

### Example 16: Identification of the Insertion Junction of the Tg Event No. 49 and Corresponding Flanking Regions

### 1. Objective

To determine the insertion locus in the genome of the sequence in Tg event no. 49 transgenic Eucalyptus and its flanking genome sequences.

### 2. Materials and Methods

### 2.1—Genomic DNA Isolation

Genomic DNA was isolated from Tg event no. 49 plants using CTAB protocol. Fresh tissue (2 g) was harvested in liquid nitrogen and ground to a fine powder. Extraction buffer (15 ml) was added (2% CTAB, 100 mM Tris pH 8, 1.5 M NaCl, 0.2 mM EDTA pH 8, 1% β-mercaptoethanol, 0.1% 1% PVP). The extract was incubated at 65° C. for 60 minutes with occasional swirling and then cooled down to room temperature. Chloroform Isoamylic Alcohol (15 ml) was added, fully mixed and centrifuged at 9000 rpm for 15 minutes at 22° C. The supernatant was transferred to a new tube and the process was repeated twice. One volume of ice-cold isopropanol was added and the tube was incubated for 30 minutes at −20° C. This was followed by centrifugation at 14000 rpm for 20 minutes at 4° C. The supernatant was discarded and 500 μl of 70% ice-cold ethanol was added to the pellet. The solution was centrifuged at 14000 rpm for 2 minutes at 4° C. and ethanol was removed. Tubes were left open in 65° C. for 30 minutes to evaporate the ethanol completely and then the pellet was re-suspended 200 μL of TE+RNase (10 ng/μL; Sigma R6513) and kept in 37° C. until fully dissolved.

### 2.2 T-DNA Structure and Insertion Site Analysis by Next Generation Sequencing (NGS)

NGS analysis was outsourced to Genewiz (https://www.genewiz.com)

Library preparation—Genomic DNA was isolated and quantified by spectrophotometer. To construct the libraries about 0.5 μg of genomic DNA was sheared to fragments of 250 bp average length, using the double strand (ds) fragmentase from the New England Biolab (NEB) Kit (M0248).

DNA sequencing—The library was sequenced on Illumina Hiseq2500 platform on one individual lane with 150-bp paired-end reads.

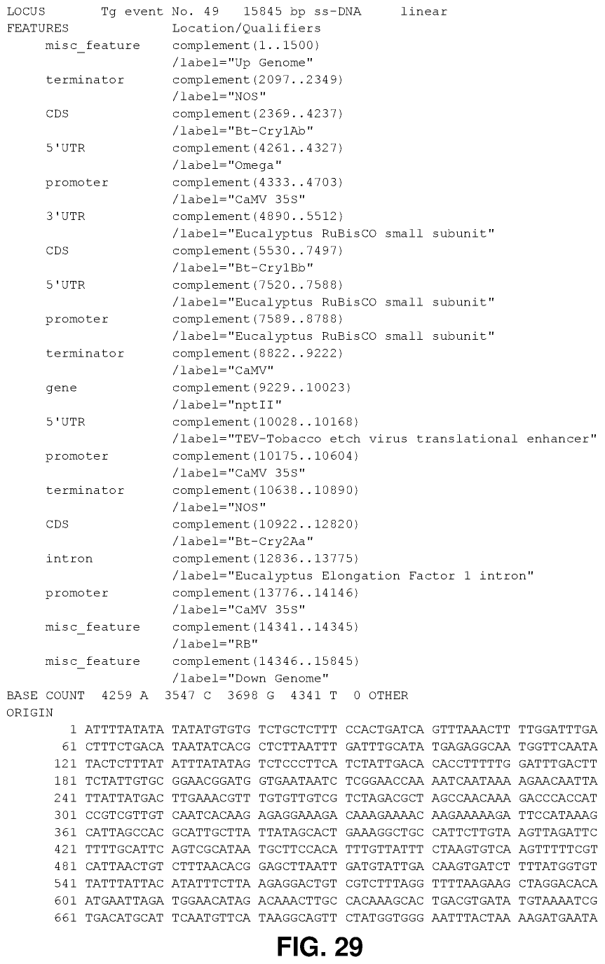

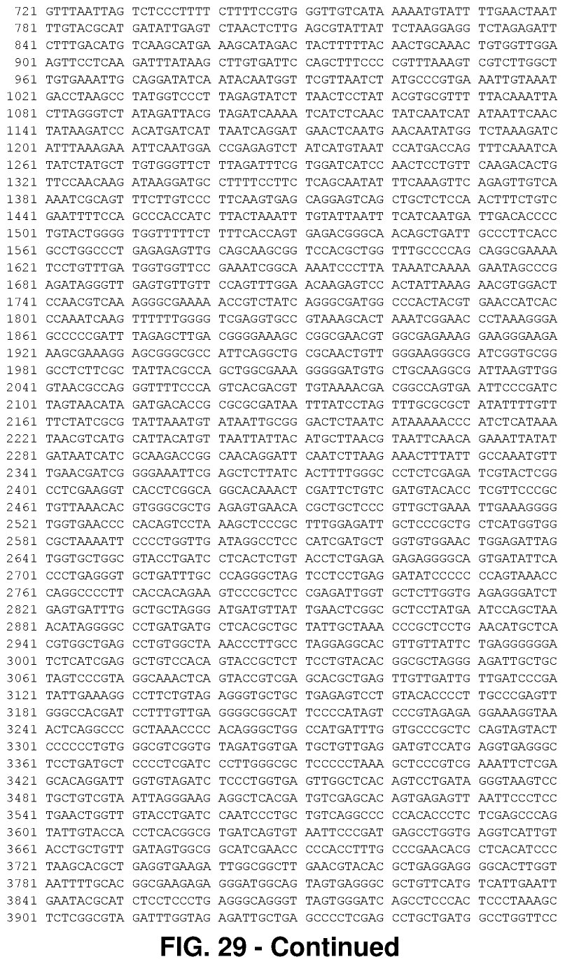

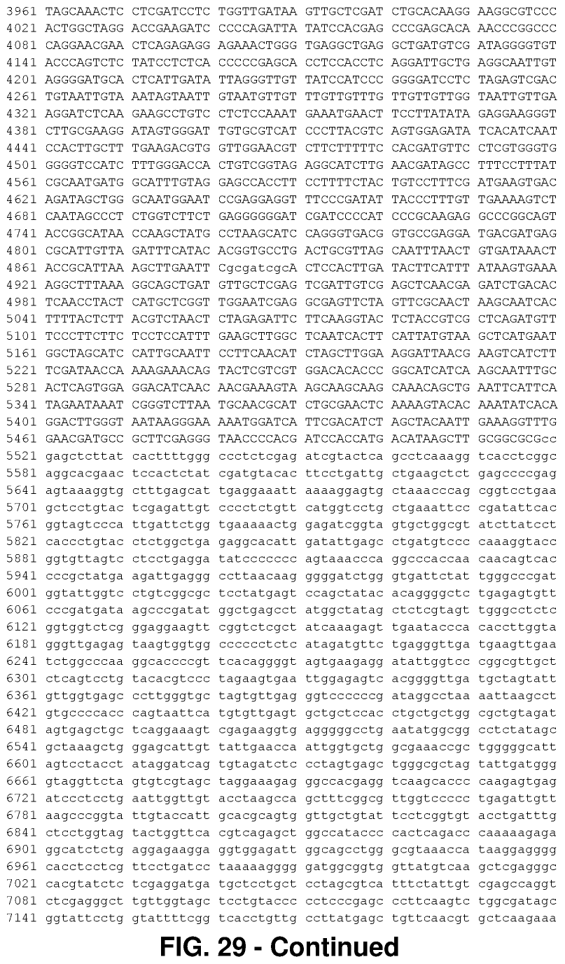

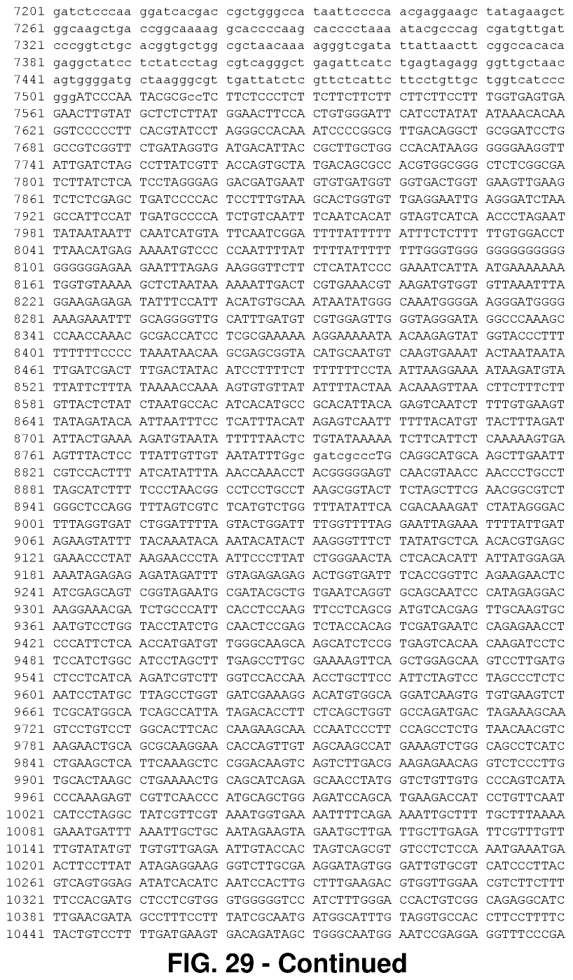

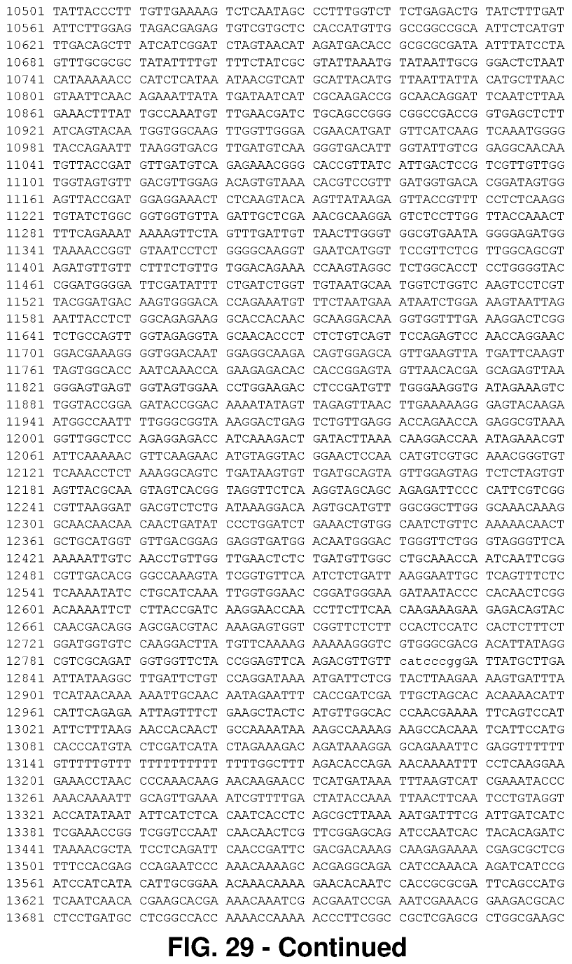

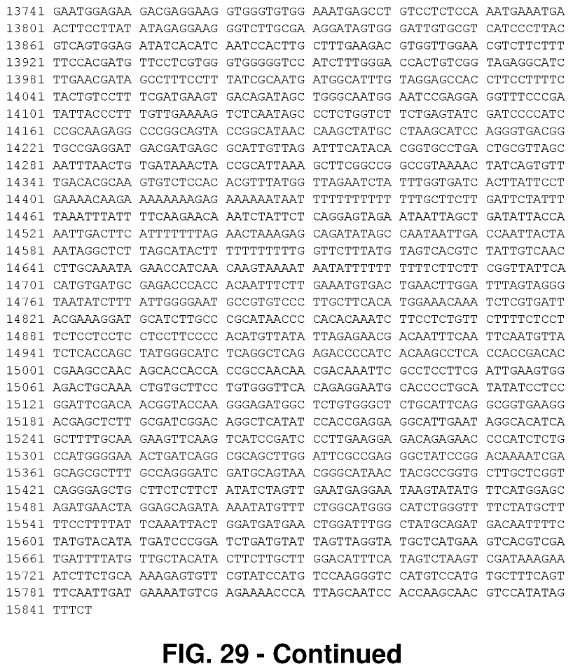

Reads mapping and analysis—Clean reads were aligned against the FGN #1521 (see FIG. 9 and FIG. 10) vector sequence (FIG. 29) using the Geneious software version 11 (http://www.geneious.com, Kearse et al., 2012). Reads that were mapped to the T-DNA sequence were used to assemble the insertion map. Reads that were mapped to both the T-DNA sequence and the genome were used to identify the location of the insert in the genome.

### 3. Results

### 3.1 T-DNA Structure in Tg Event No. 49

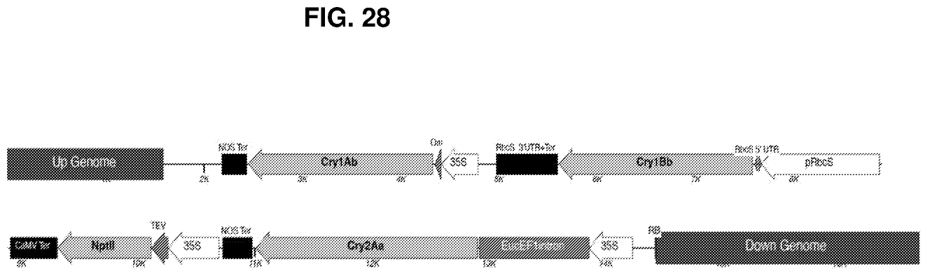

Genomic DNA was extracted from young Tg event no. 49 plants and sequenced by NGS. The NGS data analysis identified a single T-DNA insertion in the genome, in which 20 nucleotides of the RB sequence were deleted as were 71 nucleotides from the 3′ of the T-DNA including the LB sequence (FIG. 28). No reads were mapped to the backbone of the vector.

### 3.2 Insertion Site of T-DNA in Tg Event No. 49

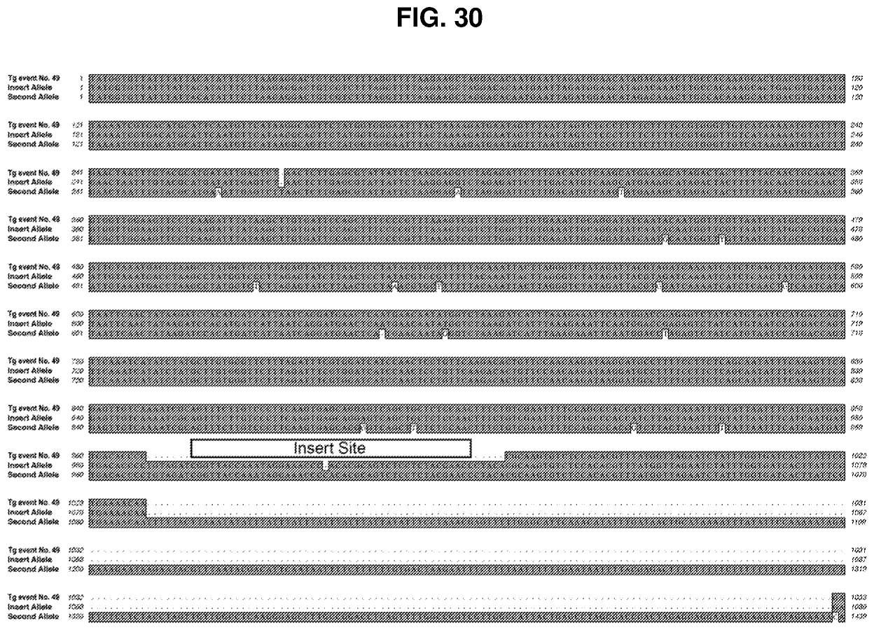

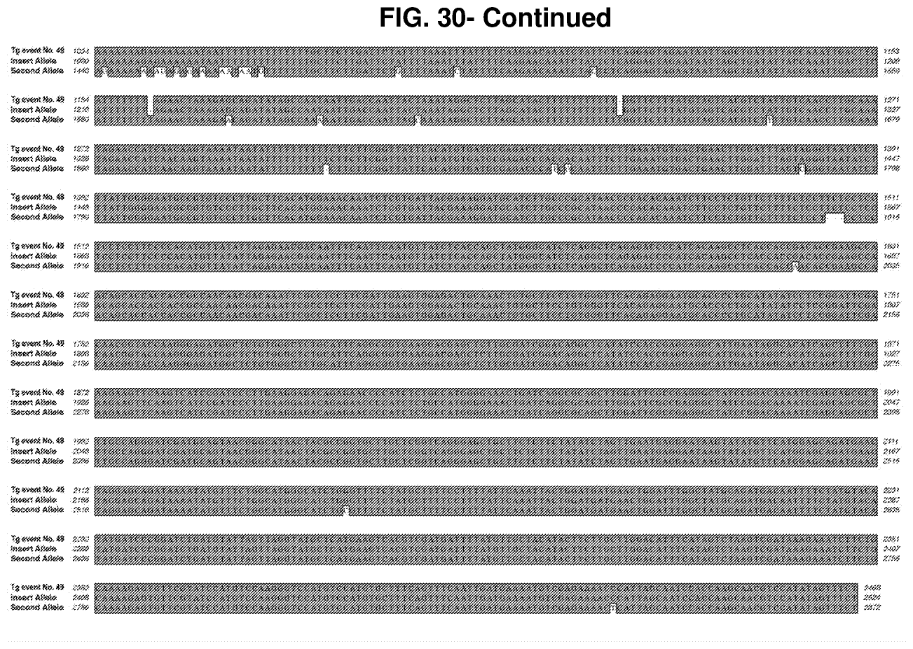

In order to identify the exact transgene integration sites, the NGS reads were mapped against FGN #1521 vector sequence (FIG. 29) to detect the insert and reads that contain both vector and genomic sequences. Minimum 15 reads were mapped to each genomic junction and based on these reads the flanking genomic sequences of 1500 nucleotides from each side were assembled. According to the NGS analysis, 57 nucleotides of genomic DNA were deleted in the insertion site as shown in FIG. 30.

A full sequence of the insert elements with genomic flanking regions is presented in FIG. 29. The full sequence is also presented as SEQ ID NO:48.

### 3.3 Genomic Location of Insertion Site and Flanking Regions of T-DNA in Tg Event No. 49

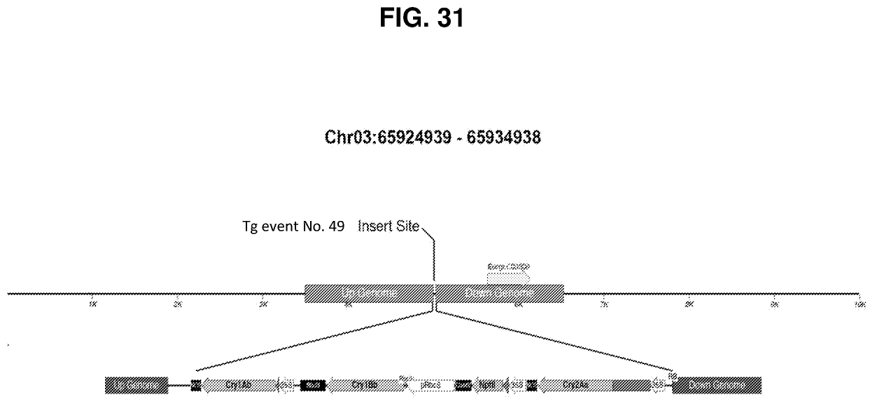

Insert flanking genomic regions of Tg event no. 49 were aligned with Eucalyptus grandis reference genome database (https://phytozome.jgi.doe.gov/pz/portal.html). The flanking regions were mapped to chromosome 3 in the genome (FIG. 31). According to the reference database, no gene was interrupted by the insert. The T-DNA was inserted 420 bps upstream to the 5′ UTR of the gene Eucgr.C03308.1

### 3.4 Second Allele in Tg Event No. 49

The sequence of the second allele of this genomic locus was assembled based on the NGS reads. The data analysis revealed a sequence difference of 349 nucleotides between the two alleles around the insertion locus (FIG. 31). This difference is the natural variation between the alleles in this genomic locus of this eucalyptus clone. Our insert is localized to the shorter allele in this region.

### Example 17: Tg Event No. 49 Event-Specific PCR and Real Time PCR Verification Assay

### 1. Objective

To develop a method for Tg event No. 49 Event-specific PCR and Real Time PCR verification.

The following describes methods useful in identifying the presence of Tg event 49 in a eucalyptus sample. A pair of PCR primers were designed for the purpose of identifying the unique junction formed between the eucalyptus genomic DNA and the inserted DNA of Tg No. event 49 in an event-specific PCR. Examples of conditions utilized for identifying the presence of Tg event No. 49 in a eucalyptus sample in an event-specific PCR are described below.

### 2. Materials, Methods and Results

### Eucalyptus Tg Event 49 Event-Specific Endpoint

### PCR—

PCR reactions mixture contained 2.5 units of Taq polymerase, 0.5 μM of each primer, 0.2 mM dNTP's and 0.5 μl genomic DNA template. Cycling conditions of PCR reactions were 3 min at 94° C., 34 cycles of 30 s at 94° C., 30 s at 55° C. and 2.5 min at 72° C., followed by a final extension step of 10 min at 72° C. PCR products were confirmed by gel electrophoresis.

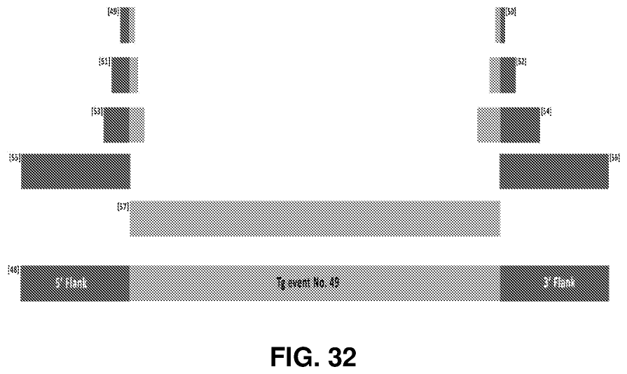

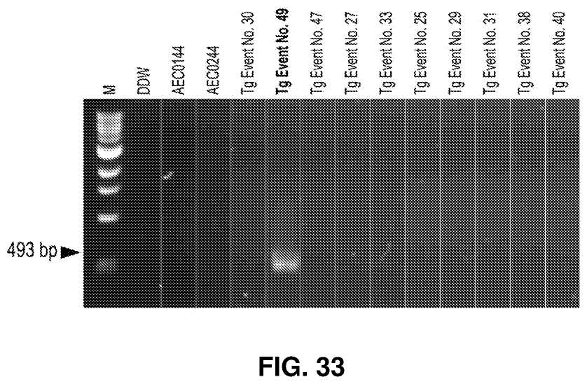

TABLE 27
PCR Primers (FIG. 32)

| 5′ Flank Border | | | | 3′ Flank Border | | | |
|---|---|---|---|---|---|---|---|
| | Amplicon | | | | Amplicon | | |
| Amplicon | expected | Forward | Reverse | Amplicon | expected | Forward | Reverse |
| Sequence | size (bp) | Primer | Primer | Sequence | size (bp) | Primer | Primer |
| SEQ ID NO: 53 | 255 | SEQ ID NO: 66 | SEQ ID NO: 67 | SEQ ID NO: 54 | 493 | SEQ ID NO: 68 | SEQ ID NO: 69 |

The sequence of the oligonucleotide forward primer (SEQ ID NO: 66) corresponds to nucleotides 1337-1356 positions on the 5′flanking region of SEQ ID NO:48 and the reverse complement primer (SEQ ID NO: 67) corresponds to nucleotides 1571-1591 on SEQ ID NO:48 near the 5′ of the insertion site. The sequence of the oligonucleotide forward primer (SEQ ID NO: 68) corresponds to positions 14228-14247 on the inserted DNA SEQ ID NO:48 of Tg event 49 near the 3′ of the insertion site and the reverse complement primer (SEQ ID NO: 69) corresponds to positions 14702-14720 on 3′flanking region of the insertion site on the nucleotide sequence SEQ ID NO:48 (FIG. 32). The primers (SEQ ID NO:66) and (SEQ ID NO: 67), can be used in a PCR assay to identify the presence of 255 bp DNA fragment (SEQ ID NO. 53) derived from Tg event 49 in a sample.

The primers (SEQ ID NO:68) and (SEQ ID NO: 69), can be used in a PCR assay to identify the presence of 493 bp DNA fragment (SEQ ID NO. 54) derived from Tg event No. 49 in a sample as indicated in FIG. 33.

### Real Time PCR—

Real time PCR was preformed using the StepOnePlus™ Real-Time PCR System, and the Fast SYBR™ Green Master Mix. 2 μl cDNA were added to each reaction, and three independent technical repeats were performed for each cDNA sample. The expression level was normalized to the eucalyptus TEF gene (Eucgr.A00744.1) and calculated using the delta-CT method.

TABLE 28
Real Time PCR Primers (FIG. 32)

| 5′ Flank Border | | | | 3′ Flank Border | | | |
|---|---|---|---|---|---|---|---|
| | Amplicon | | | | Amplicon | | |
| Amplicon | expected | Forward | Reverse | Amplicon | expected | Forward | Reverse |
| Sequence | size (bp) | Primer | Primer | Sequence | size (bp) | Primer | Primer |
| SEQ ID NO: 49 | 81 | SEQ ID NO: 58 | SEQ ID NO: 59 | SEQ ID NO: 50 | 51 | SEQ ID NO: 60 | SEQ ID NO: 61 |
| SEQ ID NO: 51 | 160 | SEQ ID NO: 62 | SEQ ID NO: 63 | SEQ ID NO: 52 | 114 | SEQ ID NO: 64 | SEQ ID NO: 65 |

Those skilled in the art will understand that various other well-known methods can be used to identify plants harbouring a recombinant DNA molecule corresponding to the insertion locus (insert and flanking sequences) of Tg event No. 49. For example, probes can be used that span, and/or target, the junction between the insert and flanking genomic sequences at either the 5′ or 3′ end of the insert.

These approaches can be readily used to identify plants of Tg event No. 49, and further plants, including progeny, off-spring, and newly created plants carrying the insertion locus (insert and genomic DNA flanking sequences) characteristic of the Tg event No. 49.

### REFERENCES

Berlitz, D. L.; Fiuza, L. M. 2004. Avaliação toxicológica de Bacillus thuringiensis aizawai para Spodoptera frugiperda (Lepidoptera: Noctuidae), em laboratório. Biociências 12(2). 115-119.
Bragança, M. A. L. et al. Effects of environmental heterogeneity on Lepidoptera and Hymenoptera populations in Eucalyptus plantations in Brazil. Forest Ecological Management.
Eizaguirre, M.; Tort, S.; López, C.; Albajes, R. Effects of sublethal concentrations of Bacillus thuringiensis on larval development of Sesamia nonagrioides. Journal of Economic Entomology, Lonham, v. 98, p. 464-470, 2005.
Ferré, Juan, and Jeroen Van Rie. “Biochemistry and genetics of insect resistance to Bacillus thuringiensis.” Annual review of entomology 47, no. 1 (2002): 501-533.
van Frankenhuyzen, K. Van. The challenge of Bacillus thuringiensis. In: Entwistle, P. F.; Cory, J. S.; Bailey, M. J.; Higgs, S. (Ed.) Bacillus thuringiensis, an environmental biopesticide: theory and practice, Chichester: J. Wiley, 1993. p. 1-23.
Gamal H. Osman, Waleed J. Altaf, Ibrahim A. S. Saleh, Raya Soltane, Hussein H. Abulreesh, Ibrahim A. Arif, Ahmed M. Ramadan, Yehia A. Osman, First report of detection of the putative receptor of Bacillus thuringiensis toxin Vip3Aa from black cutworm (Agrotis ipsilon), Saudi Journal of Biological Sciences, ISSN 1319-562X.
Gore, J.; Adamczyk, J. J.; Blanco, C. A. Selective feeding of tobacco budworm and bollworm (Lepidoptera: Noctuidae) on meridicdiet with different concentrations of 53 Bacillus thuringiensis proteins. Journal of Economic Entomology, Lanham, v. 98, n 1, p. 88-94, 2005.
Gouffon C, Van Vliet A, Van Rie J, Jansens S, Jurat-Fuentes J L. Binding sites for Bacillus thuringiensis Cry2Ae toxin on heliothine brush border membrane vesicles are not shared with Cry1A, Cry1F, or Vip3A toxin. Appl Environ Microbiol. 2011. PMID: 21441333.
Jakka, Siva, Juan Ferré, J. L. Jurat-Fuentes, M. Soberón, Y. Gao, and A. Bravo. “Cry toxin binding site models and their use in strategies to delay resistance evolution.” Bt Resistance: Characterization and Strategies for GM Crops Producing Bacillus thuringiensis Toxins, eds Soberón M, Gao Y, Bravo A (Centre for Agriculture and Biosciences International, Oxfordshire, UK) (2015): 138-149.
Kearse, M., Moir, R., Wilson, A., Stones-Havas, S., Cheung, M., Sturrock, S., Buxton, S., Cooper, A., Markowitz, S., Duran, C., Thierer, T., Ashton, B., Mentjies, P., & Drummond, A. (2012). Geneious Basic: an integrated and extendable desktop software platform for the organization and analysis of sequence data. Bioinformatics, 28(12). 1647-1649.
Lee, M. K.; Curtiss, A.; Alcantara, E. A.; DEAN, D. H. Synergistic effect of the Bacillus thuringiensis toxins Cry1Aa and Cry1Ac on the gypsy moth, Lymantria dispar. Appl. Environ. Microbiol., 62, pp. 583-586 1996.
Mascarenhas, V. J.; Luttrell, R. G. Combined effect of sublethal exposure to cotton expressing the endotoxin protein of Bacillus thuringiensis and natural enemies on 59 survival of Bollworm (Lepidoptera: Noctuidae) larvae. Environmental Entomology, College Park, v. 26, n. 4, p. 939-945, 1997.
Olson, S. A. (1994). MacVector: an integrated sequence analysis program for the Macintosh. In Computer Analysis of Sequence Data (pp. 195-201). Springer New York.
Polanczyk, R. A.; Alves, S. B. Biological parameters of Spodoptera frugiperda (J. E. Smith) (Lepidoptera: Noctuidae) assayed with Bacillus thuringiensis Berliner. Scientia Agrícola, Piracicaba, v. 62, n. 5, p. 464-468, 2005.
Wilcken, C. F. and Berti Filho, E., 2006. Biologia de Thyrinteina arnobia (Stoll) (Lepidoptera: Geometridae) em dieta artificial (I): Seleção das dietas e influência da posição da dieta artificial no desenvolvimento das lagartas. Revista de Agricultura, 81, pp. 287-300.
M. Wolfersberger, P. Luethy, A. Maurer, P. Parenti, F. V. Sacchi, B. Giordana, G. M. Hanozet. Preparation and partial characterization of amino acid transporting brush border membrane vesicles from the larval midgut of the cabbage butterfly (Pieris brassicae) Comp. Biochem. Physiol. A. Physiol., 87 (1987), pp. 301-308

1. A transgenic plant, comprising at least two of:
(i) a first nucleic acid sequence encoding a Cry2Aa Bacillus thuringiensis (Bt) pesticidal protein (PP) or an active portion thereof, and
(ii) a second nucleic acid sequence encoding a Cry1Ab Bt PP or an active portion thereof, and
(iii) a third nucleic acid sequence encoding a Cry1Bb Bt PP or an active portion thereof.

2. The transgenic plant of claim 1, comprising all three of:
(i) a first nucleic acid sequence encoding a Cry2Aa Bacillus thuringiensis (Bt) pesticidal protein (PP) or an active portion thereof, and
(ii) a second nucleic acid sequence encoding a Cry1Ab Bt PP or an active portion thereof, and
(iii) a third nucleic acid sequence encoding a Cry1Bb Bt PP or an active portion thereof.

3. The transgenic plant of claim 1, wherein:
said Cry2Aa Bt PP is characterized by comprising a sequence with at least 95% sequence identity to SEQ ID NO: 1,
said Cry1Ab Bt PP is characterized by comprising a sequence with at least 95% sequence identity to SEQ ID NO: 3, and
said Cry1Bb Bt PP is characterized by comprising a sequence with at least 95% sequence identity to SEQ ID NO: 5.

4. (canceled)

5. The transgenic plant of claim 1, wherein:
said nucleic acid sequence encoding said Cry2Aa Bt PP is characterized by comprising a sequence with at least 95% sequence identity to SEQ ID NO: 2,
said nucleic acid sequence encoding said Cry1Ab Bt PP is characterized by comprising a sequence with at least 95% sequence identity to SEQ ID NO: 4, and
said nucleic acid sequence encoding said Cry1Bb Bt PP is characterized by comprising a sequence with at least 95% sequence identity to SEQ ID NO: 6

6. (canceled)

7. (canceled)

8. The transgenic plant of claim 1, further comprising the nucleic acid sequence of a Rubisco promoter comprising a sequence with at least 90% sequence identity to the Rubisco promoter of SEQ ID NO: 36, operably linked to at least one of the first, second or third nucleic acid sequences.

9. (canceled)

10. (canceled)

11. (canceled)

12. (canceled)

13. The transgenic plant of claim 1, wherein expression of the at least two, or all three Bt PP confers increased resistance to insect pest infestation relative to that in a plant that does not express the at least two, or all least 3, Bt PP respectively.

14. The transgenic plant of claim 13, wherein combined expression of the at least two, or all three Bt PP respectively, produces synergistic effect on increasing resistance to insect pest infestation.

15. (canceled)

16. (canceled)

17. (canceled)

18. A seed, tissue, or plant material from the transgenic plant according to claim 1, wherein said seed comprises said nucleic acid sequences encoding at least two, or all three, of the Cry2Aa, Cry1Ab, and Cry1Bb Bt PP.

19. (canceled)

20. A method of inhibiting growth of, or killing, or managing an insect pest infestation of a plant, comprising transgenically co-expressing in the plant of claim 1 at least two, or all three, of Cry2Aa, Cry1Ab and Cry1Bb Bacillus thuringiensis-derived Pesticidal Proteins (Bt PP).

21. A method for producing a plant that is resistant to insect pest infestation, the method comprising producing the plant of claim 1 by transforming the plant with nucleic acids encoding at least two, or all three of Cry2Aa, Cry1Ab and Cry1Bb Bacillus thuringiensis-derived Pesticidal Proteins (Bt PP).

22. A method for producing a progeny plant that is resistant to insect pest infestation, the method comprising at least one of:
a) propagating a first plant as defined in claim 1 to produce the progeny plant, and
b) crossing a first plant of claim 1 with second plant to produce the progeny plant,
c) wherein the progeny plant comprises the recited nucleic acids from the first plant of claim 1.

23. A method of controlling insect pest infestation the method comprising growing the plant of claim 1 in the field.

24. The method of claim 23, wherein said insect pest infestation is caused by an insect pest is selected from the group consisting of: Thyrinteina arnobia (Geometridae), Physocleora dukinfeldia, Sarsina violascens (Erebidae), Glena spp. (Geometridae), Melanolophia consimilaria (Geometridae), Eacles spp. (Satumiidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

25. The method of claim 24, wherein said insect pest is selected from the group consisting of: Thyrinteina arnobia (Geometridae), Thyrinteina leucocerae (Geometridae), Physocleora dukinfeldia, Sarsina violascens (Eribidae), Oxydia vesulia (Geometridae), Melanolophia consimilaria (Geometridae), and Spodoptera cosmioides (Noctuidae).

26. The method of claim 25, wherein said insect pest is Thyrinteina arnobia (Geometridae) or Physocleora dukinfeldia (Geometridae).

27. The method of claim 20, wherein said plant is a woody plant.

28. The method of claim 27, wherein said plant is a Eucalyptus plant.

29. (canceled)

30. A nucleic acid construct, comprising:
(i) a first nucleic acid comprising the sequence of the Rubisco promoter of SEQ ID NO: 36 or a sequence having at least 80% sequence similarity to the Rubisco promoter of SEQ ID NO: 36;
(ii) a second nucleic acid sequence encoding at least one of the Bt PP selected from the group consisting of Cry1Bb, Cry1Ab and Cry2Aa,
wherein said first nucleic acid and said second nucleic acid are operably linked.

31. The nucleic acid construct of claim 30, wherein said Bt PP is characterized by comprising a sequence with at least 95% sequence identity to one of the amino acid sequences selected from the group consisting of SEQ ID NO: 5, SEQ ID NO: 3 or SEQ ID NO: 1.

32. A nucleic acid construct, comprising at least two, or at least three, of:
(i) a first nucleic acid sequence encoding Cry2Aa Bt PP,
(ii) a second nucleic acid sequence encoding Cry1Ab Bt PP, and
(iii) a third nucleic acid sequence encoding Cry1Bb Bt PP,
wherein expression of said first nucleic acid, said second nucleic acid and said third nucleic acid are directed by a promoter that is functional in a plant cell.

33. The nucleic acid construct of claim 32, wherein:
said first nucleic acid is characterized by comprising a sequence with at least 95% sequence identity to SEQ ID NO: 2,
said second nucleic acid is characterized by comprising a sequence with at least 95% sequence identity to SEQ ID NO: 4, and
said third nucleic acid is characterized by comprising a sequence with at least 95% sequence identity to SEQ ID NO: 6.

34. The nucleic acid construct of claim 32, wherein:
a) said first nucleic acid comprises the sequence of SEQ ID NO: 2,
b) said second nucleic acid comprises the sequence of SEQ ID NO: 4, and
c) said third nucleic acid comprises the sequence of SEQ ID NO: 6.

35. A method of making a transgenic plant, comprising:
a) introducing at least one nucleic acid construct of claim 30 into plant cells to produce transformed plant cells, and
b) culturing the transformed plant cells under conditions appropriate to regenerate a plant, thereby making a transgenic plant.

36. (canceled)

37. A recombinant DNA molecule comprising a nucleotide sequence selected from the group consisting of SEQ ID NO:48, SEQ ID NO:49, SEQ ID NO:50, SEQ ID NO:51, SEQ ID NO:52, SEQ ID NO:53 and SEQ ID NO:54 and a complete complement thereof of any of the foregoing.

38. The recombinant DNA molecule of claim 37, wherein said molecule is from eucalyptus Tg event No:49.

39. A DNA molecule comprising a polynucleotide segment of sufficient length to function as a DNA probe that hybridizes specifically under stringent hybridization conditions with eucalyptus Tg event No:49 DNA in a sample, wherein detecting hybridization of said DNA molecule under said stringent hybridization conditions is diagnostic for the presence of eucalyptus Tg event No:49 DNA in said sample.

40. The DNA molecule of claim 39, wherein said sample comprises a plant, part thereof, tissue thereof or cell thereof, from eucalyptus Tg event No:49.

41. A pair of DNA molecules, comprising a first DNA molecule and a second DNA molecule different from the first DNA molecule, that function as DNA primers when used together in an amplification reaction with a sample containing a plant, part thereof or tissue thereof, of or from eucalyptus Tg event No:49 template DNA to produce an amplicon diagnostic for the presence of said eucalyptus Tg event No:49 DNA in said sample, wherein said amplicon comprises the recombinant DNA molecule of claim 37.

42. The pair of DNA molecules of claim 41, wherein:
a) The first DNA molecule comprises the sequence of any one of SEQ ID NO: 58, SEQ ID NO: 60, SEQ ID NO: 62, SEQ ID NO: 64, SEQ ID NO: 66, SEQ ID NO: 68, and
b) The second DNA molecule comprises the sequence of any one of SEQ ID NO: 59, SEQ ID NO: 61, SEQ ID NO: 63, SEQ ID NO: 65, SEQ ID NO: 67, SEQ ID NO: 69.

43. A method of detecting the presence of a DNA segment diagnostic for eucalyptus Tg event No:49 DNA in a sample, said method comprising:
a) contacting said sample with the DNA molecule of claim 39;
b) subjecting said sample and said DNA molecule to stringent hybridization conditions; and
c) detecting hybridization of said DNA molecule to said DNA in said sample,
wherein said detection is diagnostic for the presence of said eucalyptus Tg event No:49 DNA in said sample.

44. A method of detecting the presence of a DNA segment diagnostic for eucalyptus Tg event No:49 DNA in a sample, said method comprising:
a) contacting said sample with the pair of DNA molecules of claim 41;
b) performing an amplification reaction sufficient to produce a DNA amplicon; and
c) detecting the presence of said DNA amplicon in said reaction,
wherein said DNA amplicon comprises the nucleotide sequence selected from the group consisting of SEQ ID NO:48, SEQ ID NO:49, SEQ ID NO:50, SEQ ID NO:51, SEQ ID NO:52, SEQ ID NO:53 and SEQ ID NO:54.

45. A eucalyptus plant, part thereof, tissue thereof or cell thereof comprising eucalyptus Tg event No:49 DNA characterized by the detectable presence of the recombinant DNA molecule of claim 37.

46. The eucalyptus plant, part thereof, tissue thereof or cell thereof of claim 45, wherein the plant, plant part, or plant cell, or part thereof is insecticidal when provided in the diet of an insect pest.

47. The eucalyptus plant, part thereof, tissue thereof or cell thereof of claim 45, wherein the insect pest is selected from the group consisting of Thyrinteina arnobia (Geometridae), Physocleora dukinfeldia (Geometridae), Sarsina violascens (Erebidae), Glena spp. (Geometridae), Melanolophia consimilaria (Geometridae), Eacles spp. (Satumiidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

48. (canceled)

49. The eucalyptus plant of claim 46, wherein the plant is further defined as progeny of any generation of a plant comprising the eucalyptus Tg event No:49.

50. A method for protecting a eucalyptus plant from insect infestation, wherein said method comprises providing in the diet of an insect pest an insecticidally effective amount of cells or tissue of the plant of claim 46.

51. The method of claim 50, wherein said insect pest is selected from the group consisting of Thyrinteina arnobia (Geometridae), Physocleora dukinfeldia (Geometridae), Sarsina violascens (Erebidae), Glena spp. (Geometridae), Melanolophia consimilaria (Geometridae), Eacles spp. (Satumiidae), Eupseudosoma aberrans (Arctiidae), Eupseudosoma involuta (Arctiidae), Euselasia apisaon (Riodinidae), Nystalea nyseus (Notodontidae), Spodoptera cosmioides (Noctuidae), Thyrinteina leucocerae (Geometridae), Oxydia vesulia (Geometridae), or Iridopsis spp. (Geometridae).

52. (canceled)

53. A method of producing an insect resistant eucalyptus plant comprising:
a) breeding two different eucalyptus plants with at least one of the two different eucalyptus plants comprising the recombinant DNA molecule of claim 37 to produce progeny;
b) confirming in said progeny the presence of the recombinant DNA molecule; and
c) selecting said progeny comprising the recombinant DNA molecule;
wherein said progeny of step c) are insect resistant.

54. A eucalyptus plant part or tissue comprising a detectable amount of the recombinant DNA molecule of claim 37.

55. A nonliving eucalyptus plant material comprising a detectable amount of the DNA molecule of claim 37.
